SEQUENCE LISTING (297 sequences)

SEQ ID NO: 1 - protein, 68 aa, Arabidopsis thaliana
MATATTTATAAFSGVVSVGTETRRIYSFSHLQPSAAFPAKPSSFKSLKLKQSARLTRRLDHRPFVVRC

SEQ ID NO: 2 - protein, 68 aa, synthetic construct
  source 1..68: note = Recombinant
MASSTTTATAAFSGVVSVGTETRRIYSFSHLQPSAAFPAKPSSFKSLKLKQSARLTRRLDHRPFVVRC

SEQ ID NO: 3 - protein, 35 aa, unidentified
  source 1..35: note = Gossypium raimondii
MLNIAPSCVLASGISKPVTKMASTENKDDHSSAKR

SEQ ID NO: 4 - protein, 45 aa, Canavalia rosea
MVAVFNDVVFPPSQTLLRPSFHSPTFFFSSPTPKFTRTRPNRILR

SEQ ID NO: 5 - protein, 60 aa, Senna obtusifolia
MPAIAMASLTDLPSLSPTQTLVHSNTSFISSRTCFVCPIIPFPSRSQLNRRIACIRSNVR

SEQ ID NO: 6 - protein, 55 aa, unidentified
  source 1..55: note = Nicotiana benthamiana
MTTTPVANHPNIFTHRSPPSSSSSSPSAFLTRTSFLPFSSICKRNSVNCNGWRTR

SEQ ID NO: 7 - protein, 34 aa, Brassica napus
MDFSLLRPASTQPFLSPFSNPFPRSRPYKPLNLR

SEQ ID NO: 8 - protein, 37 aa, Adansonia digitata
MAILIDLSLLRSSPSVFSFSKPNHRIPPRIYKPFKLR

SEQ ID NO: 9 - protein, 47 aa, unidentified
  source 1..47: note = cultivar = osiana; note = Rosa hybrida
MTTLSRLADLPSFAAPPPLLTHRPPPSVFLTPKPTKPSPPHHFFKLR

SEQ ID NO: 10 - protein, 46 aa, Xanthium strumarium
MSSLTDLPSLNHYRTCSPRPFPISRQTSSSINPNNLTTSNRWRRFR

SEQ ID NO: 11 - protein, 35 aa, Conyza canadensis
MTSLTNFTPLKLTNPNYLNTTTTYNHRKLSNFRFR

SEQ ID NO: 12 - protein, 45 aa, Amaranthus palmeri
MSAMALSSSILQCPPHSDISFRFSAYTATRSPFFFGRPRKLSYIH

SEQ ID NO: 13 - protein, 47 aa, Spinacia oleracea
MSAMALSSTMALSLPQSSMSLSHCRHNRITILIPSSSLRRRGGSSIR

SEQ ID NO: 14 - protein, 61 aa, Kochia scoparia
MSAMASPSIIPQSFLQRSPTSLQSRSNYSKNHIIISISTPCSHGKNQRRFLRKTTHFRSIH

SEQ ID NO: 15 - protein, 34 aa, Setaria italica
MVAAAMATAPSAGVPPLRGTRGPARFRIRGVSVR

SEQ ID NO: 16 - protein, 27 aa, Allium cepa
MATTTAAAAVTISIPKKPVFIRRPRLR

SEQ ID NO: 17 - protein, 33 aa, Digitaria sanguinalis
MLSSTATASSASSHHPYRSASARASSTRLRPVL

SEQ ID NO: 18 - protein, 49 aa, Amaranthus palmeri
MVIQSITHLSPKLALPSPLSISAKNYPVAVMGNISEREEPTSAKRVAVV

SEQ ID NO: 19 - protein, 48 aa, Spinacia oleracea
MVILPVSQLSTNLGLSLVSPTKNNPVMGNVSERNQVNQPISAKRVAVV

SEQ ID NO: 20 - protein, 51 aa, Sedum album
MLSLSSSHSSATTYSLRQRYSTTTKGSLNQPEMASAENPSSKGSGKRGAVV

SEQ ID NO: 21 - protein, 38 aa, Camelina sativa
MELSLLRPSTQSLLPSFSKPNLRLHVYKPLKLRCSVAG

SEQ ID NO: 22 - protein, 43 aa, Cucumis melo
MATGATLLTDLPFRRPHPLTLLRPSDIPSFYPLHISLQNNRLR

SEQ ID NO: 23 - protein, 30 aa, Eragrostis tef
MVAAAATMATAAPPLRAPQTLARPRRGSVR

SEQ ID NO: 24 - protein, 32 aa, Canavalia rosea
MYVSPASNNPRACLKLSQEMASSAADGNPRSV

SEQ ID NO: 25 - protein, 42 aa, Eragrostis tef
MLSSAATASSASAHPYRPASARASRSVLAMAGSDDTRAAPAR

SEQ ID NO: 26 - protein, 41 aa, Andropogon gerardii
MVAATAMATAASAAAPLLNGTRRPARLRHRGLRVRCAAVAG

SEQ ID NO: 27 - protein, 24 aa, Digitaria sanguinalis
MLSSTATASSASSHHPYRSASARA

SEQ ID NO: 28 - protein, 23 aa, Eragrostis tef
MLSSAATASSASAHPYRPASARA

SEQ ID NO: 29 - protein, 28 aa, Amaranthus graecizans
MSAMALSSSILQCPPHSDISFRFFAHTR

SEQ ID NO: 30 - protein, 13 aa, Ambrosia trifida
MASPTIVDNQKPA

SEQ ID NO: 31 - protein, 14 aa, Brassica napus
MASNAAADHDKLSG

SEQ ID NO: 32 - protein, 13 aa, unidentified
  source 1..13: note = cultivar = osiana; note = Rosa hybrida
MASPSPGDKHSSV

SEQ ID NO: 33 - protein, 55 aa, Amaranthus cruentus
MKGRKRRITRESAREMSAMALSSSILQCPPHSDISFRFSAHSPTHSPIFFGRPRK

SEQ ID NO: 34 - protein, 45 aa, Taraxacum officinale
MTYLTDVGSLNCYRSWPSLPAPGTVGALTSKNPRYLITYGPAHRK

SEQ ID NO: 35 - protein, 67 aa, Canavalia rosea
MVAVFNDVVFPPSQTLLRPSFHSPTFFFSSPTPKFTRTRPNRILRCSIAQESTTSPSQSRESAPLDC

SEQ ID NO: 36 - protein, 52 aa, Eragrostis tef
MVAAAATMATAAPPLRAPQTLARPRRGSVRCAVVSDAAEAPAAPGARLSADC

SEQ ID NO: 37 - protein, 51 aa, Allium cepa
MATTTAAAAVTISIPKKPVFIRRPRLRCSAVASDAIISNEAPTGTTISADC

SEQ ID NO: 38 - protein, 77 aa, Taraxacum officinale
MTYLTDVGSLNCYRSWPSLPAPGTVGALTSKNPRYLITYGPAHRKCNSWRFRCSIAKDSPITPPISNESNSQPLLDC

SEQ ID NO: 39 - protein, 71 aa, Cucumis melo
MATGATLLTDLPFRRPHPLTLLRPSDIPSFYPLHISLQNNRLRSHFRCSIAEGSTALSPSNASSQSSILDC

SEQ ID NO: 40 - protein, 71 aa, Xanthium strumarium
MSSLTDLPSLNHYRTCSPRPFPISRQTSSSINPNNLTTSNRWRRFRCSIANDTPISPPISSDSTSHPFLDC

SEQ ID NO: 41 - protein, 38 aa, Camelina sativa; identical to SEQ ID NO: 21

SEQ ID NO: 42 - protein, 45 aa, Taraxacum officinale; identical to SEQ ID NO: 34

SEQ ID NO: 43 - protein, 65 aa, Taraxacum officinale
MTYLTDVGSLNCYRSWPSLPAPGTVGALTSKNPRYLITYGPAHRKDSPITPPISNESNSQPLLDC

SEQ ID NO: 44 - protein, 60 aa, Senna obtusifolia
MPAIAIASLTDLPSLSPTQTLVHSNTSFISSRTCFVCPIIPFPSRSQLNRRIACIRSNVR

SEQ ID NO: 45 - protein, 19 aa, Eragrostis tef
MVAAAEAPAAPGARLSADC

SEQ ID NO: 46 - protein, 25 aa, Allium cepa
MATTTASDAIISNEAPTGTTISADC

SEQ ID NO: 47 - protein, 12 aa, Allium cepa
MATTGTTISADC

SEQ ID NO: 48 - protein, 19 aa, Cucumis melo
MATALSPSNASSQSSILDC

SEQ ID NO: 49 - protein, 32 aa, Xanthium strumarium
MSSLTDLPSLNHYRTCSPPISSDSTSHPFLDC

SEQ ID NO: 50 - DNA, 204 bp, synthetic construct
  source 1..204: mol_type = other DNA; note = Recombinant
atggccaccgccaccactaccgccaccgctgcgttctccggcgtggtgagcgtcggcactgagacgcgcaggatctactccttcagccacctccagccttctgctgcgttccccgctaagccgtcttcgttcaagagcctgaagctgaaacagtccgcacgccttacccggcgcctggaccataggccattcgttgtcaggtgc

SEQ ID NO: 51 - DNA, 204 bp, synthetic construct
  source 1..204: mol_type = other DNA; note = Recombinant
atggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgt

SEQ ID NO: 52 - DNA, 204 bp, synthetic construct
  source 1..204: mol_type = other DNA; note = Recombinant
atggcttcctccacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgt

SEQ ID NO: 53 - DNA, 105 bp, synthetic construct
  source 1..105: mol_type = other DNA; note = Recombinant
atgcttaacattgcgccgagttgtgttttggccagcgggatctctaagcccgtgaccaagatggctagcacggagaacaaggacgaccacagcagcgccaagagg

SEQ ID NO: 54 - DNA, 135 bp, synthetic construct
  source 1..135: mol_type = other DNA; note = Recombinant
atggtggctgtgttcaacgacgtagtgttccctccttcgcagacccttcttcgcccctccttccacagcccgacgttcttttttagcagccccacaccaaagttcacgcgtacgaggccgaatagaatactgcgg

SEQ ID NO: 55 - DNA, 180 bp, synthetic construct
  source 1..180: mol_type = other DNA; note = Recombinant
atgccggcgatagcaatggcttctttaactgatctgccgtcgttgagccccacacagaccctcgttcactcgaacacgagcttcatttcatcgagaacctgcttcgtctgtccgatcatccccttcccatcgaggtcgcaactgaaccgccgcatcgcctgcatcaggtccaacgtaagg

SEQ ID NO: 56 - DNA, 165 bp, synthetic construct
  source 1..165: mol_type = other DNA; note = Recombinant
atgaccacaaccccggtagcaaaccaccccaatatcttcactcaccgaagccctccgtcatcttcctcgtcctcacccagcgcgtttctgacccgcacctcctttctgcccttctctagcatctgcaaaaggaactctgtgaactgcaatgggtggcgaacccgg

SEQ ID NO: 57 - DNA, 102 bp, synthetic construct
  source 1..102: mol_type = other DNA; note = Recombinant
atggacttcagtctccttaggcccgcttcgacgcagccgttcctctcacccttctccaatcccttcccacggagtaggccatacaagccacttaatctgagg

SEQ ID NO: 58 - DNA, 111 bp, synthetic construct
  source 1..111: mol_type = other DNA; note = Recombinant
atggccatcttgattgacctctccctcctgaggtcctctccgtcggtcttctccttctccaagccgaaccacaggataccaccgcggatctacaagccgttcaagttgagg

SEQ ID NO: 59 - DNA, 141 bp, synthetic construct
  source 1..141: mol_type = other DNA; note = Recombinant
atgaccacgctttccaggctcgctgaccttccttcttttgctgcccctcctcctctcttgacccaccggccccctccttcagttttcctgactccgaagccgacaaagccgtcacctccacatcacttctttaaactgcgc

SEQ ID NO: 60 - DNA, 138 bp, synthetic construct
  source 1..138: mol_type = other DNA; note = Recombinant
atgtcgtccctaacggacctcccctccctgaatcactataggacgtgcagcccgcgcccattccccatctccaggcagaccagttcatcaattaacccaaacaacttgacgaccagtaaccgttggcgcaggttcagg

SEQ ID NO: 61 - DNA, 105 bp, synthetic construct
  source 1..105: mol_type = other DNA; note = Recombinant
atgacgagtctcaccaacttcaccccgctcaagctgacgaaccccaactacctcaacacgaccaccacctacaaccaccgtaagctctccaacttccggttccgc

SEQ ID NO: 62 - DNA, 135 bp, synthetic construct
  source 1..135: mol_type = other DNA; note = Recombinant
atgtcggccatggcgctgtccagcagcattctacagtgcccgcctcactcagacatatccttccgcttctcggcatacactgccacccgctcacctttcttcttcgggaggccaaggaaactatcttacatccac

SEQ ID NO: 63 - DNA, 141 bp, synthetic construct
  source 1..141: mol_type = other DNA; note = Recombinant
atgtcggccatggcattgagctccaccatggccctcagcctgccacaatctagcatgtccttgagccactgcagacacaatagaataactattctgatcccctcgagctcgttacggcgacggggaggttcctcgatccgc

SEQ ID NO: 64 - DNA, 183 bp, synthetic construct
  source 1..183: mol_type = other DNA; note = Recombinant
atgtctgctatggcgagcccctccatcatcccgcagtcgttcctccagcgaagcccgacctccttgcaatctcgatccaactactcgaagaaccacatcatcatctccatcagcaccccgtgctctcatgggaagaaccagcgacgtttcttgcgaaagaccacccacttccgatccatccac

SEQ ID NO: 65 - DNA, 102 bp, synthetic construct
  source 1..102: mol_type = other DNA; note = Recombinant
atggtcgccgctgcaatggctacagccccttccgctggagtccctcctcttagagggacaaggggtccagcaaggtttagaatccggggagtgtcagtgcgt

SEQ ID NO: 66 - DNA, 81 bp, synthetic construct
  source 1..81: mol_type = other DNA; note = Recombinant
atggccactaccacagcagccgcggcggtcaccatcagcattcctaaaaagcctgtttttatccgccgcccacgacttcgt

SEQ ID NO: 67 - DNA, 99 bp, synthetic construct
  source 1..99: mol_type = other DNA; note = Recombinant
atgttgtctagcactgctactgcaagttctgcatcctcacaccacccctaccgttcagcttctgcaagggcttcgtcgacacgtctccgcccggtcctt

SEQ ID NO: 68 - DNA, 147 bp, synthetic construct
  source 1..147: mol_type = other DNA; note = Recombinant
atggtcattcagtcaattacgcatctttctcccaagctcgcactgccctctccgctgtcgatctcggctaagaactacccggtggccgtgatggggaatatcagcgagagggaggagccaacttctgctaaaagggtggccgtggtg

SEQ ID NO: 69 - DNA, 144 bp, synthetic construct
  source 1..144: mol_type = other DNA; note = Recombinant
atggtcattctacccgtgtcccagctctcgactaatttggggctttcccttgttagtccaacgaagaacaacccggtgatgggcaacgtgtccgagaggaaccaggtgaaccagccaatctccgccaagcgcgttgctgtcgtg

SEQ ID NO: 70 - DNA, 153 bp, synthetic construct
  source 1..153: mol_type = other DNA; note = Recombinant
atgctctcactgagcagctcccactcatccgcgacaacgtattctctccggcaacggtactctacaacgaccaaaggttcgttgaaccagcctgagatggccagcgccgaaaacccttccagcaagggatcaggtaagagaggagcagtggtg

SEQ ID NO: 71 - DNA, 114 bp, synthetic construct
  source 1..114: mol_type = other DNA; note = Recombinant
atggagctgagcctcctaagaccgtctactcagtcattgctcccctcgttcagcaagcctaatttgcggctccacgtgtacaagccccttaagctccgatgcagcgtagccggt

SEQ ID NO: 72 - DNA, 129 bp, synthetic construct
  source 1..129: mol_type = other DNA; note = Recombinant
atggcgacaggagccaccctgctaacagacctgccgttccgtaggccgcacccgcttacgctcttacgtccgagcgatatcccgtccttttacccactacacataagcctacagaacaatcgtttgagg

SEQ ID NO: 73 - DNA, 90 bp, synthetic construct
  source 1..90: mol_type = other DNA; note = Recombinant
atggtggctgctgcggcaacgatggctaccgccgcaccaccattaagagcgcctcaaactcttgcacgaccgcgaagaggtagtgtgaga

SEQ ID NO: 74 - DNA, 96 bp, synthetic construct
  source 1..96: mol_type = other DNA; note = Recombinant
atgtatgtgtcgcccgcctcgaacaacccacgagcatgcctcaagctgtcacaggaaatggcgtcttcagcagcagacggcaacccaagatccgtt

SEQ ID NO: 75 - DNA, 126 bp, synthetic construct
  source 1..126: mol_type = other DNA; note = Recombinant
atgttgtctagcgcagcgacagctagcagcgcaagtgctcatccttatcgacctgcttctgcccgggcgagtaggagcgtgttggctatggctggatcagacgatactagggcagctcctgcccgg

SEQ ID NO: 76 - DNA, 123 bp, synthetic construct
  source 1..123: mol_type = other DNA; note = Recombinant
atggtggctgcgaccgcaatggccaccgctgcttcggctgctgcgcctctcctaaacggaacgagacgaccggcacgattgagacatagaggtttacgtgttaggtgtgctgcagtagcagga

SEQ ID NO: 77 - DNA, 72 bp, synthetic construct
  source 1..72: mol_type = other DNA; note = Recombinant
atgctttctagcactgccacagcttcctcagcttctagccaccacccgtatcgttcagcttcggcacgtgcc

SEQ ID NO: 78 - DNA, 69 bp, synthetic construct
  source 1..69: mol_type = other DNA; note = Recombinant
atgcttagctcagcagctacggcctctagtgcttctgcccatccataccgtcccgcatctgctcgagca

SEQ ID NO: 79 - DNA, 84 bp, synthetic construct
  source 1..84: mol_type = other DNA; note = Recombinant
atgagcgcgatggcgctttcttctagcatcttgcaatgccccccccactctgacatttctttccgcttcttcgcccacactcgc

SEQ ID NO: 80 - DNA, 39 bp, synthetic construct
  source 1..39: mol_type = other DNA; note = Recombinant
atggcgagtcccacgatcgttgacaaccagaagccagcg

SEQ ID NO: 81 - DNA, 42 bp, synthetic construct
  source 1..42: mol_type = other DNA; note = Recombinant
atggctagtaacgccgctgctgaccacgataagctctcgggt

SEQ ID NO: 82 - DNA, 39 bp, synthetic construct
  source 1..39: mol_type = other DNA; note = Recombinant
atggcgtcgccgtccccaggcgacaaacattcgtctgta

SEQ ID NO: 83 - DNA, 165 bp, synthetic construct
  source 1..165: mol_type = other DNA; note = Recombinant
atgaaggggcggaagagacggatcacgcgggagtctgcaagggagatgtcagcgatggcattgtcttcgagcatactccagtgccctcctcactccgacatctctttccgttttagcgctcactcaccgacacacagccctatcttctttgggcgtcccaggaaa

SEQ ID NO: 84 - DNA, 135 bp, synthetic construct
  source 1..135: mol_type = other DNA; note = Recombinant
atgacctacctcactgatgtgggtagtctcaattgctacaggtcctggcctagcctaccggcccctgggacggtcggagcattgacttctaagaacccccgctacttgatcacatacggtccggctcaccgaaag

SEQ ID NO: 85 - DNA, 201 bp, synthetic construct
  source 1..201: mol_type = other DNA; note = Recombinant
atggtggctgtgttcaacgacgtagtgttccctccttcgcagacccttcttcgcccctccttccacagcccgacgttcttttttagcagccccacaccaaagttcacgcgtacgaggccgaatagaatactgcggtgctcgattgcgcaggagtctacaacatcgccgtcgcagtcgcgagagtcagctccactcgattgt

SEQ ID NO: 86 - DNA, 156 bp, synthetic construct
  source 1..156: mol_type = other DNA; note = Recombinant
atggtggctgctgcggcaacgatggctaccgccgcaccaccattaagagcgcctcaaactcttgcacgaccgcgaagaggtagtgtgagatgtgccgtcgttagcgatgctgcagaagctccggctgctcctggcgctagactctctgcagattgc

SEQ ID NO: 87 - DNA, 153 bp, synthetic construct
  source 1..153: mol_type = other DNA; note = Recombinant
atggccactaccacagcagccgcggcggtcaccatcagcattcctaaaaagcctgtttttatccgccgcccacgacttcgttgctcggcagttgcatccgacgcaatcatctccaacgaggcccctacagggacgacaatctcggctgactgt

SEQ ID NO: 88 - DNA, 231 bp, synthetic construct
  source 1..231: mol_type = other DNA; note = Recombinant
atgacctacctcactgatgtgggtagtctcaattgctacaggtcctggcctagcctaccggcccctgggacggtcggagcattgacttctaagaacccccgctacttgatcacatacggtccggctcaccgaaagtgcaacagctggcgcttccggtgctctattgcaaaggactcccccatcacgcccccaatttcgaacgagagcaattcacagcccctgctagactgc

SEQ ID NO: 89 - DNA, 213 bp, synthetic construct
  source 1..213: mol_type = other DNA; note = Recombinant
atggcgacaggagccaccctgctaacagacctgccgttccgtaggccgcacccgcttacgctcttacgtccgagcgatatcccgtccttttacccactacacataagcctacagaacaatcgtttgaggagtcatttcaggtgctcaatcgccgagggctcgacggcactgagcccatctaacgcatcgtcgcaatcgagtatcttggactgc

SEQ ID NO: 90 - DNA, 213 bp, synthetic construct
  source 1..213: mol_type = other DNA; note = Recombinant
atgtcgtccctaacggacctcccctccctgaatcactataggacgtgcagcccgcgcccattccccatctccaggcagaccagttcatcaattaacccaaacaacttgacgaccagtaaccgttggcgcaggttcaggtgctctattgcgaacgacaccccgatcagcccgccgatttccagcgactctacttcccaccctttcttggactgt

SEQ ID NO: 91 - DNA, 114 bp, synthetic construct
  source 1..114: mol_type = other DNA; note = Recombinant
atggagctaagcctcctaagaccgtctactcagtcattgctcccctcgttcagcaagcctaatttgcggctccacgtgtacaagccccttaagctccgatgcagcgtagccggt

SEQ ID NO: 92 - DNA, 135 bp, synthetic construct
  source 1..135: mol_type = other DNA; note = Recombinant
atgacctacctcactgatgtgggtagtctcaattgctacaggtcttggcctagcctaccggcccctgggacggtcggagcattgacttctaagaacccccgctacttgatcacatacggtccggctcaccgaaag

SEQ ID NO: 93 - DNA, 195 bp, synthetic construct
  source 1..195: mol_type = other DNA; note = Recombinant
atgacctacctcactgatgtgggtagtctcaattgctacaggtcctggcctagcctaccggcccctgggacggtcggagcattgacttctaagaacccccgctacttgatcacatacggtccggctcaccgaaaggactcccccatcacgcccccaatttcgaacgagagcaattcacagcccctgctagactgc

SEQ ID NO: 94 - DNA, 180 bp, synthetic construct
  source 1..180: mol_type = other DNA; note = Recombinant
atgccggcgatagcaatagcttctttaactgatctgccgtcgttgagccccacacagaccctcgttcactcgaacacgagcttcatttcatcgagaacctgcttcgtctgtccgatcatccccttcccatcgaggtcgcaactgaaccgccgcatcgcctgcatcaggtccaacgtaagg

SEQ ID NO: 95 - DNA, 57 bp, synthetic construct
  source 1..57: mol_type = other DNA; note = Recombinant
atggtggctgctgcggaagctccggctgctcctggcgctagactctctgcagattgc

SEQ ID NO: 96 - DNA, 75 bp, synthetic construct
  source 1..75: mol_type = other DNA; note = Recombinant
atggccactaccacagcatccgacgcaatcatctccaacgaggcccctacagggacgacaatctcggctgactgt

SEQ ID NO: 97 - DNA, 36 bp, synthetic construct
  source 1..36: mol_type = other DNA; note = Recombinant
atggccactacagggacgacaatctcggctgactgt

SEQ ID NO: 98 - DNA, 57 bp, synthetic construct
  source 1..57: mol_type = other DNA; note = Recombinant
atggcgacggcactgagcccatctaacgcatcgtcgcaatcgagtatcttggactgc

SEQ ID NO: 99 - DNA, 96 bp, synthetic construct
  source 1..96: mol_type = other DNA; note = Recombinant
atgtcgtccctaacggacctcccctccctgaatcactataggacgtgcagcccgccgatttccagcgactctacttcccaccctttcttggactgt

SEQ ID NO: 100 - protein, 179 aa, Enterobacter cloacae [sequence omitted]

SEQ ID NO: 101 - protein, 178 aa, Pantoea ananatis
MKALILFSTRDGQTQKIASAIADEIKGQQSCDVINIQDAKTLDWQQYDRVLIGASIRYGHFQPVVNEFVKHNLLALQQRVSGFFSVNLTARKPEKRSPETNAYTVKFLAQSPWQPDCCAVFAGALYYPRYRWFDRVMIQFIMRMTGGETDASKEVEYTDWQQVQRFARDFAQLPGKSY

SEQ ID NO: 102 - protein, 177 aa, Pantoea stewartii
MKALILYSTRDGQTRKIASSIADVIRQQQQCDVLNIKDASLPDWAQYDRVLIGASIRYGHFQPVVDKFVKQHLHELQQRTSGFFSVNLTARKPEKRSPETNAYTQKFLAHSPWQPDCCAVFAGALYYPRYRWFDRVMIQLIMRMTGGETDSTKEVEYTDWQQVSTFANDFAQLPGKS

SEQ ID NO: 103 - protein, 181 aa, Escherichia coli
MKTLILFSTRDGQTREIASYLASELKELGIQADVANVHRIEEPQWENYDRVVIGASIRYGHYHSAFQEFVKKHATRLNSMPSAFYSVNLVARKPEKRTPQTNSYARKFLMNSQWRPDRCAVIAGALRYPRYRWYDRFMIKLIMKMSGGETDTRKEVVYTDWEQVANFAREIAHLTDKPTLK

SEQ ID NO: 104 - protein, 178 aa, Erwinia toletana
MKALILFSSREGQTREIASYIANSIKEEMECDVFNILRVEQIDWSQYDRVLIGGSIHYGHFHPAVAKFVKRHLHELQQRSSGFFCVNLTARKADKRTPQTNAYMRKFLLQSPWQPDCCAVFAGALRYTRYRWFDRVMIQLIMRMTGGETDTSKEVEYTDWTQVARFAQEFAHLPGKTQ

SEQ ID NO: 105 - protein, 179 aa, Pectobacterium carotovorum
MKALIVFSSRDGQTRAIASYIANTLKGTLECDVVNVLNANDIDLSQYDRVAIGASIRYGRFHPAVNQFIRKHLTSLQQLPSAFFSVNLTARKPEKRTIQTNAYTRKFLLNSPWQPDLCCVFAGALRYPRYRWFDRVMIQLIMRITGGETDSTKEIEYTDWQQVARFAQDFAQLAAKNPA

SEQ ID NO: 106 - protein, 179 aa, Shimwellia blattae
MKTLILFSTRDGQTHKIARHIAGVLEEQGKACELVDLLQPGEPDWSTVECVVLGASIRYGHFHKSFIRFVNTHAQRLNNMPGALFTVNLVARKPEKQSPQTNSYTRKFLAASPWQPQRCQVFAGALRYPRYSWYDRMMIRLIMKMAGGETDTRKEVEYTDWQSVTRFAREIAQLPGETR

SEQ ID NO: 107 - protein, 178 aa, Pantoea stewartii
MKALILFSSRDGQTQLIASSIAKELEGKQACDVLNILDTTNVEWTQYDRVLIGASIRYGHFHPAVAEFVKRHQRELQQRSSGFFSVNLTARKPEKRSPETNAYTAKFLNQSPWQPDCCAVFAGALRYPRYRWFDRIMIQLIMRMTGGETDSSKEVEYTDWQQVTRFAQEFARLPGKTS

SEQ ID NO: 108 - protein, 180 aa, Enterobacter cloacae
MKTLILFSTRDGQTREIAAFLASELKEQGIYADVINLNRTEEIAWQEYDRVVIGASIRYGHFHPAVDRFVKKHTETLNSLPGAFFSVNLVARKAEKRTPQTNSYTRKFLLNSPWKPAACAVFAGALRYPRYRWYDRFMIRLIMKMTGGETDTRKEVVYTDWSQVASFAREIVQLTRSSRL

SEQ ID NO: 109 - protein, 177 aa, Enterobacter mori
MKILILFSTRDGQTREIAASLASELKEQAFDVDVVNLHRAENIAWEEYDGVVIGASIRYGHFHSTLNSFVKKHQQALKKLPGAFYSVNLVARKPEKRTPQTNSYTRKFLLDSPWQPDLSAVFAGALRYPRYNWYDRIMIRLIMKITGGETDTRKEVVYTDWQQVTHFAHEIVQLVRK

SEQ ID NO: 110 - protein, 178 aa, Enterobacter cloacae
KALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGGETDTSKEVEYTDWEQVKKFAEDFAKLSYKKAL

SEQ ID NO: 111 - protein, 177 aa, Pantoea ananatis
KALILFSTRDGQTQKIASAIADEIKGQQSCDVINIQDAKTLDWQQYDRVLIGASIRYGHFQPVVNEFVKHNLLALQQRVSGFFSVNLTARKPEKRSPETNAYTVKFLAQSPWQPDCCAVFAGALYYPRYRWFDRVMIQFIMRMTGGETDASKEVEYTDWQQVQRFARDFAQLPGKSY

SEQ ID NO: 112 - protein, 180 aa, Escherichia coli
KTLILFSTRDGQTREIASYLASELKELGIQADVANVHRIEEPQWENYDRVVIGASIRYGHYHSAFQEFVKKHATRLNSMPSAFYSVNLVARKPEKRTPQTNSYARKFLMNSQWRPDRCAVIAGALRYPRYRWYDRFMIKLIMKMSGGETDTRKEVVYTDWEQVANFAREIAHLTDKPTLK

SEQ ID NO: 113 - protein, 177 aa, Erwinia toletana
KALILFSSREGQTREIASYIANSIKEEMECDVFNILRVEQIDWSQYDRVLIGGSIHYGHFHPAVAKFVKRHLHELQQRSSGFFCVNLTARKADKRTPQTNAYMRKFLLQSPWQPDCCAVFAGALRYTRYRWFDRVMIQLIMRMTGGETDTSKEVEYTDWTQVARFAQEFAHLPGKTQ

SEQ ID NO: 114 - protein, 178 aa, Pectobacterium carotovorum
KALIVFSSRDGQTRAIASYIANTLKGTLECDVVNVLNANDIDLSQYDRVAIGASIRYGRFHPAVNQFIRKHLTSLQQLPSAFFSVNLTARKPEKRTIQTNAYTRKFLLNSPWQPDLCCVFAGALRYPRYRWFDRVMIQLIMRITGGETDSTKEIEYTDWQQVARFAQDFAQLAAKNPA

SEQ ID NO: 115 - protein, 178 aa, Shimwellia blattae
KTLILFSTRDGQTHKIARHIAGVLEEQGKACELVDLLQPGEPDWSTVECVVLGASIRYGHFHKSFIRFVNTHAQRLNNMPGALFTVNLVARKPEKQSPQTNSYTRKFLAASPWQPQRCQVFAGALRYPRYSWYDRMMIRLIMKMAGGETDTRKEVEYTDWQSVTRFAREIAQLPGETR

SEQ ID NO: 116 - protein, 179 aa, Enterobacter cloacae
KTLILFSTRDGQTREIAAFLASELKEQGIYADVINLNRTEEIAWQEYDRVVIGASIRYGHFHPAVDRFVKKHTETLNSLPGAFFSVNLVARKAEKRTPQTNSYTRKFLLNSPWKPAACAVFAGALRYPRYRWYDRFMIRLIMKMTGGETDTRKEVVYTDWSQVASFAREIVQLTRSSRL

SEQ ID NO: 117 - protein, 178 aa, synthetic construct
  source 1..178: note = Recombinant
KALVLYSTRDGQTHAIASYIASCMKEKAECDVIDLTHGEHVNLTQYDQVLIGANIRYGHFNAVLDKFIKRNVDQLNNMPSAFFCVNLTARKPEKRTPQTNPYVRKFLLATPWQPALCGVFAGALRYPRYRWIDKVMIQLIMRMTGGETDTSKEVEYTDWEQVKKFAEDFAKLSYKKAL

SEQ ID NO: 118 - protein, 172 aa, synthetic construct
  source 1..172: note = Recombinant
KALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGGETDTSKEVEYTDWEQVKKFAEDFAKL

SEQ ID NO: 119 - protein, 179 aa, synthetic construct
  source 1..179: note = Recombinant
KTLILFSTRDGQTREIAAFLASELKEQGIYADVINLNRTEEIAWQEYDRVVIGASIRYGHFHPAVDRFVKKHTETLNSLPGAFFSVNLVARKAEKRTPQTNSYTRKFLLNSPWKPAACAVFAGALRYPRYRWYDRFMIRLIMKMTGGETDTRKEVVYTDWSQIASFAREIVQLTRSSRL

SEQ ID NO: 120 - protein, 504 aa, Amaranthus tuberculatus
MGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 121 - DNA, 537 bp, synthetic construct
  source 1..537: mol_type = other DNA; note = Recombinant
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcagctgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 122 - DNA, 534 bp, synthetic construct
  source 1..534: mol_type = other DNA; note = Recombinant
atgaaagcgctgattctgtttagcacccgcgatggccagacccagaaaattgcgagcgcgattgcggatgaaattaaaggccagcagagctgcgatgtgattaacattcaggatgcgaaaaccctggattggcagcagtatgatcgcgtgctgattggcgcgagcattcgctatggccattttcagccggtggtgaacgaatttgtgaaacataacctgctggcgctgcagcagcgcgtgagcggcttttttagcgtgaacctgaccgcgcgcaaaccggaaaaacgcagcccggaaaccaacgcgtataccgtgaaatttctggcgcagagcccgtggcagccggattgctgcgcggtgtttgcgggcgcgctgtattatccgcgctatcgctggtttgatcgcgtgatgattcagtttattatgcgcatgaccggcggcgaaaccgatgcgagcaaagaagtggaatataccgattggcagcaggtgcagcgctttgcgcgcgattttgcgcagctgccgggcaaaagctat

SEQ ID NO: 123 - DNA, 531 bp, synthetic construct
  source 1..531: mol_type = other DNA; note = Recombinant
atgaaagcgctgattctgtatagcacccgcgatggccagacccgcaaaattgcgagcagcattgcggatgtgattcgccagcagcagcagtgcgatgtgctgaacattaaagatgcgagcctgccggattgggcgcagtatgatcgcgtgctgattggcgcgagcattcgctatggccattttcagccggtggtggataaatttgtgaaacagcatctgcatgaactgcagcagcgcaccagcggcttttttagcgtgaacctgaccgcgcgcaaaccggaaaaacgcagcccggaaaccaacgcgtatacccagaaatttctggcgcatagcccgtggcagccggattgctgcgcggtgtttgcgggcgcgctgtattatccgcgctatcgctggtttgatcgcgtgatgattcagctgattatgcgcatgaccggcggcgaaaccgatagcaccaaagaagtggaatataccgattggcagcaggtgagcacctttgcgaacgattttgcgcagctgccgggcaaaagc

SEQ ID NO: 124 - DNA, 546 bp, Escherichia coli
  source 1..546: mol_type = genomic DNA
gtgaaaacattaattcttttctcaacaagggacggacaaacgcgcgagattgcctcctacctggcttcggaactgaaagaactggggatccaggcggatgtcgccaatgtgcaccgcattgaagaaccacagtgggaaaactatgaccgtgtggtcattggtgcttctattcgctatggtcactaccattcagcgttccaggaatttgtcaaaaaacatgcgacgcggctgaattcgatgccgagcgccttttactccgtgaatctggtggcgcgcaaaccggagaagcgtactccacagaccaacagctacgcgcgcaagtttctgatgaactcgcaatggcgtcccgatcgctgcgcggtcattgccggggcgctgcgttacccacgttatcgctggtacgaccgttttatgatcaagctgattatgaagatgtcaggcggtgaaacggatacgcgcaaagaagttgtctataccgattgggagcaggtggcgaatttcgcccgagaaatcgcccatttaaccgacaaaccgacgctgaaataa

SEQ ID NO: 125 - DNA, 534 bp, synthetic construct
  source 1..534: mol_type = other DNA; note = Recombinant
atgaaagcgctgattctgtttagcagccgcgaaggccagacccgcgaaattgcgagctatattgcgaacagcattaaagaagaaatggaatgcgatgtgtttaacattctgcgcgtggaacagattgattggagccagtatgatcgcgtgctgattggcggcagcattcattatggccattttcatccggcggtggcgaaatttgtgaaacgccatctgcatgaactgcagcagcgcagcagcggctttttttgcgtgaacctgaccgcgcgcaaagcggataaacgcaccccgcagaccaacgcgtatatgcgcaaatttctgctgcagagcccgtggcagccggattgctgcgcggtgtttgcgggcgcgctgcgctatacccgctatcgctggtttgatcgcgtgatgattcagctgattatgcgcatgaccggcggcgaaaccgataccagcaaagaagtggaatataccgattggacccaggtggcgcgctttgcgcaggaatttgcgcatctgccgggcaaaacccag

SEQ ID NO: 126 - DNA, 537 bp, synthetic construct
  source 1..537: mol_type = other DNA; note = Recombinant
atgaaagcgctgattgtgtttagcagccgcgatggccagacccgcgcgattgcgagctatattgcgaacaccctgaaaggcaccctggaatgcgatgtggtgaacgtgctgaacgcgaacgatattgatctgagccagtatgatcgcgtggcgattggcgcgagcattcgctatggccgctttcatccggcggtgaaccagtttattcgcaaacatctgaccagcctgcagcagctgccgagcgcgttttttagcgtgaacctgaccgcgcgcaaaccggaaaaacgcaccattcagaccaacgcgtatacccgcaaatttctgctgaacagcccgtggcagccggatctgtgctgcgtgtttgcgggcgcgctgcgctatccgcgctatcgctggtttgatcgcgtgatgattcagctgattatgcgcattaccggcggcgaaaccgatagcaccaaagaaattgaatataccgattggcagcaggtggcgcgctttgcgcaggattttgcgcagctggcggcgaaaaacccggcg

SEQ ID NO: 127 - DNA, 537 bp, synthetic construct
  source 1..537: mol_type = other DNA; note = Recombinant
atgaagaccttgatcctattctccaccagggacggccaaacacacaagatcgcaaggcacatcgcaggagtcctcgaagagcaggggaaggcctgcgagttggtcgatctgttacagcccggcgaaccagactggagtaccgttgaatgcgtcgttctaggggccagcattagatatggtcacttccataagtctttcatcaggttcgtaaacactcacgcgcagcgcttgaataatatgccaggcgcccttttcacagttaacttagtcgcccgaaagcccgagaagcagagtccacagacgaactcttacacccgcaagtttctcgccgcctccccttggcagccacagcgatgccaagttttcgcgggcgctttgaggtaccctaggtactcgtggtacgacagaatgatgatacgtttgataatgaagatggccgggggcgagactgacacaaggaaggaggttgagtacactgactggcagtcggtgactcggttcgcgagggagatcgctcagctgccgggagagacgcgg

SEQ ID NO: 128 - DNA, 534 bp, synthetic construct
  source 1..534: mol_type = other DNA; note = Recombinant
atgaaagcgctgattctgtttagcagccgcgatggccagacccagctgattgcgagcagcattgcgaaagaactggaaggcaaacaggcgtgcgatgtgctgaacattctggataccaccaacgtggaatggacccagtatgatcgcgtgctgattggcgcgagcattcgctatggccattttcatccggcggtggcggaatttgtgaaacgccatcagcgcgaactgcagcagcgcagcagcggcttttttagcgtgaacctgaccgcgcgcaaaccggaaaaacgcagcccggaaaccaacgcgtataccgcgaaatttctgaaccagagcccgtggcagccggattgctgcgcggtgtttgcgggcgcgctgcgctatccgcgctatcgctggtttgatcgcattatgattcagctgattatgcgcatgaccggcggcgaaaccgatagcagcaaagaagtggaatataccgattggcagcaggtgacccgctttgcgcaggaatttgcgcgcctgccgggcaaaaccagc

SEQ ID NO: 129 - DNA, 540 bp, synthetic construct
  source 1..540: mol_type = other DNA; note = Recombinant
atgaaaaccctgattctgtttagcacccgcgatggccagacccgcgaaattgcggcgtttctggcgagcgaactgaaagaacagggcatttatgcggatgtgattaacctgaaccgcaccgaagaaattgcgtggcaggaatatgatcgcgtggtgattggcgcgagcattcgctatggccattttcatccggcggtggatcgctttgtgaaaaaacataccgaaaccctgaacagcctgccgggcgcgttttttagcgtgaacctggtggcgcgcaaagcggaaaaacgcaccccgcagaccaacagctatacccgcaaatttctgctgaacagcccgtggaaaccggcggcgtgcgcggtgtttgcgggcgcgctgcgctatccgcgctatcgctggtatgatcgctttatgattcgcctgattatgaaaatgaccggcggcgaaaccgatacccgcaaagaagtggtgtataccgattggagccaggtggcgagctttgcgcgcgaaattgtgcagctgacccgcagcagccgcctg

SEQ ID NO: 130 - DNA, 531 bp, synthetic construct
  source 1..531: mol_type = other DNA; note = Recombinant
atgaaaattctgattctgtttagcacccgcgatggccagacccgcgaaattgcggcgagcctggcgagcgaactgaaagaacaggcgtttgatgtggatgtggtgaacctgcatcgcgcggaaaacattgcgtgggaagaatatgatggcgtggtgattggcgcgagcattcgctatggccattttcatagcaccctgaacagctttgtgaaaaaacatcagcaggcgctgaaaaaactgccgggcgcgttttatagcgtgaacctggtggcgcgcaaaccggaaaaacgcaccccgcagaccaacagctatacccgcaaatttctgctggatagcccgtggcagccggatctgagcgcggtgtttgcgggcgcgctgcgctatccgcgctataactggtatgatcgcattatgattcgcctgattatgaaaattaccggcggcgaaaccgatacccgcaaagaagtggtgtataccgattggcagcaggtgacccattttgcgcatgaaattgtgcagctggtgcgcaaa

SEQ ID NO: 131 - DNA, 540 bp, synthetic construct
  source 1..540: mol_type = other DNA; note = Recombinant
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 132 - DNA, 537 bp, synthetic construct
  source 1..537: mol_type = other DNA; note = Recombinant
atgaaggccttgatcctgttctctacacgcgacggacagacacagaagatcgcatctgccatcgctgatgagataaaggggcagcaatcgtgcgacgtgattaacatacaggatgccaaaaccctcgactggcagcagtacgaccgggtactcatcggcgcctccattcgttacgggcatttccagcccgttgtgaatgagtttgtcaagcacaacctcttggccctacagcagagagtttccggattcttctccgtgaacttgacagcccgaaagccagagaagcggagccccgagactaacgcttatacagtcaaattcttggcgcagtcaccctggcaaccggactgctgcgctgtttttgcgggggccctgtactacccacggtaccggtggttcgatagggtgatgatacagttcataatgcgaatgacggggggagagaccgacgcatcgaaagaggtggagtacactgactggcagcaggtgcagcggttcgcgcgagacttcgcgcagttaccgggtaagtcctactga

SEQ ID NO: 133 - DNA, 534 bp, synthetic construct
  source 1..534: mol_type = other DNA; note = Recombinant
atgaaggcgctgatcttgtactcaaccagggacggtcagactcgcaagattgcaagtagcattgcggacgtcatcaggcagcagcagcagtgcgacgtcttaaacattaaagacgcatcacttcctgactgggcccaatatgaccgagtgctcatcggagctagcatccgttacgggcatttccagcccgttgtagacaagttcgtgaagcagcacttgcacgagcttcagcagcggacctccggcttcttctccgtgaacctgacggcgaggaagcctgaaaaaaggagccctgagaccaatgcctacacccagaaattcttggcgcactccccttggcagcccgattgctgtgccgttttcgcgggggccctttactaccccaggtaccgttggttcgaccgggtgatgatccagttgattatgcgcatgactggtggagagaccgactctaccaaggaagtggagtacactgactggcagcaggtgagtaccttcgccaacgattttgcccagcttccaggcaagagctaa

SEQ ID NO: 134 - DNA, 546 bp, synthetic construct
  source 1..546: mol_type = other DNA; note = Recombinant
atgaagaccttgattctattctccacaagggacggccagactagggagatcgcttcctacctggccagcgagctaaaggagcttggcattcaggcagacgtggctaacgtgcaccgaattgaggagccgcagtgggagaactacgatcgggtcgtgatcggcgccagcatccggtatggacactaccacagcgcgttccaggagttcgtgaaaaagcacgcgacccgtctgaatagcatgccatcagcgttctactcggtcaacctcgtggctcgtaagcccgagaagcggacaccccagaccaactcgtatgccaggaagttccttatgaactcgcagtggcgaccggaccgctgcgcggtgatcgccggtgcgctcaggtaccctcgttataggtggtacgacaggtttatgattaaacttataatgaaaatgagcggcggagagaccgacaccagaaaagaggtggtttacacagactgggagcaggtagcaaacttcgctagggagattgctcacctcaccgacaagccgaccttgaagtaa

SEQ ID NO: 135 - DNA, 537 bp, synthetic construct
  source 1..537: mol_type = other DNA; note = Recombinant
atgaaggcccttatactgttcagttccagagaaggccagacgagggagatagcgagttacattgccaactcgataaaggaggaaatggaatgcgacgtgttcaacatccttcgtgtggagcagatcgactggtctcaatacgaccgcgtcctgatcgggggctcgatacactacggccatttccacccagcggtggcaaaatttgtcaagaggcacctccatgagttgcaacagaggtcttccggctttttctgcgtcaacctgacggccaggaaggccgacaagcggactccccagaccaatgcctacatgagaaagttcttgttgcagtccccatggcaacccgattgctgcgccgtgtttgcgggggcccttaggtacacccgttacaggtggttcgacagggtaatgattcagctgatcatgaggatgacgggcggagagactgacacatcgaaggaggtggagtacacagactggacgcaggtcgcccgcttcgcgcaggagttcgcccatttgcccggcaaaactcagtga

SEQ ID NO: 136 - DNA, 540 bp, synthetic construct
  source 1..540: mol_type = other DNA; note = Recombinant
atgaaggctcttatcgtattctcttcgagggatggccaaacccgagcgatcgcgtcttatattgctaataccctcaaagggaccctagagtgcgacgtcgtcaacgtcctcaatgctaacgacattgatttgagccagtacgaccgtgtggccattggcgcctccattcgctacgggaggttccacccagctgttaaccagtttatccggaagcaccttacgagcctccagcagctaccatctgcgttcttctccgtgaacctcacagctcggaagcccgagaagaggactatacaaaccaacgcgtacactaggaagtttctactgaactcgccgtggcagccggacctgtgctgcgtgttcgcgggagcccttcgctatccccgttacaggtggtttgaccgagtgatgattcaactcataatgcgcataacggggggcgagacagactccaccaaggagatcgagtacaccgactggcagcaggtcgcgcgattcgcccaggattttgcacagcttgccgcaaagaacccggcatga

SEQ ID NO: 137 - DNA, 540 bp, synthetic construct
  source 1..540: mol_type = other DNA; note = Recombinant
atgaagaccttgatcctattctccaccagggacggccaaacacacaagatcgcaaggcacatcgcaggagtcctcgaagagcaggggaaggcctgcgagttggtcgatctgttacagcccggcgaaccagactggagtaccgttgaatgcgtcgttctaggggccagcattagatatggtcacttccataagtctttcatcaggttcgtaaacactcacgcgcagcgcttgaataatatgccaggcgcccttttcacagttaacttagtcgcccgaaagcccgagaagcagagtccacagacgaactcttacacccgcaagtttctcgccgcctccccttggcagccacagcgatgccaagttttcgcgggcgctttgaggtaccctaggtactcgtggtacgacagaatgatgatacgtttgataatgaagatggccgggggcgagactgacacaaggaaggaggttgagtacactgactggcagtcggtgactcggttcgcgagggagatcgctcagctgccgggagagacgcggtag

SEQ ID NO: 138 - DNA, 537 bp, synthetic construct
  source 1..537: mol_type = other DNA; note = Recombinant
atgaaggccctaattttattcagtagtagggacggccagacccagcttatagcatcgtctatcgccaaggagctcgaagggaagcaggcgtgcgacgtgttgaatatcctcgacacgactaatgtggagtggacccagtacgaccgcgtgctgattggagcatccatccggtacgggcactttcaccctgcggtcgccgagttcgtaaagcgtcaccagcgagagctacagcagagaagtagtggctttttctctgtgaacttgacggcccgtaagccggaaaagaggtcccccgagactaacgcctataccgccaagttccttaaccaaagtccatggcagcctgactgttgcgctgtgttcgctggggctttgcgataccctcggtaccgctggttcgacagaattatgatccagctaatcatgcggatgactgggggtgagacagattcttcaaaggaggtcgagtacaccgactggcagcaggtgacccgcttcgcgcaagagttcgccaggcttccgggaaagaccagttga

SEQ ID NO: 139 - DNA, 543 bp, synthetic construct
  source 1..543: mol_type = other DNA; note = Recombinant
atgaagaccctaatactgttctctacccgcgacgggcagacaagggagatcgccgcgttccttgcctcggagctgaaggagcaggggatttacgctgacgtcataaaccttaaccggacggaggagatagcttggcaggagtatgatagagtcgtaatcggggcgtcgatccgatacgggcatttccaccctgctgtcgaccgcttcgtgaagaagcacacagagacactcaactcactgcccggcgcctttttctctgtaaaccttgttgcccggaaagccgagaagagaacgccgcagacgaactcatacaccaggaagttcctattaaacagcccgtggaagccagcggcctgcgcggtctttgctggggccctccgctaccctagataccgctggtacgacaggttcatgatacgactgattatgaaaatgacaggcggggagacggatacccgaaaggaggtagtctacactgactggtcgcaggtcgcgtcgtttgccagagagatagtccagttgaccaggtcatcgcgcttgtga

SEQ ID NO: 140 - DNA, 534 bp, synthetic construct
  source 1..534: mol_type = other DNA; note = Recombinant
atgaagatattaatccttttctccacccgtgacggccaaacccgtgagattgcggcgtccttggcgtccgaactcaaggagcaggcattcgacgtggacgtcgtcaaccttcaccgggccgagaacatcgcatgggaggagtacgacggtgttgtcatcggagcgtccatcaggtacggccactttcatagtaccctgaactcatttgtcaagaagcatcagcaggctcttaagaagcttcccggggctttctacagcgtgaacctcgtcgcccggaagcctgagaagcgcacaccgcagaccaatagctacacccgcaagttcctcttggattccccgtggcagcccgacctttcagccgtgttcgccggggcactcaggtaccctcggtacaattggtacgaccgtatcatgattagacttatcatgaagattacaggcggcgagactgataccaggaaggaagtagtctacacagactggcagcaggtcactcactttgctcacgagatcgtccagctcgtgcggaagtag

SEQ ID NO: 141 - DNA, 537 bp, synthetic construct
  source 1..537: mol_type = other DNA; note = Recombinant
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 142 - DNA, 534 bp, synthetic construct
  source 1..534: mol_type = other DNA; note = Recombinant
aaggccttgatcctgttctctacacgcgacggacagacacagaagatcgcatctgccatcgctgatgagataaaggggcagcaatcgtgcgacgtgattaacatacaggatgccaaaaccctcgactggcagcagtacgaccgggtactcatcggcgcctccattcgttacgggcatttccagcccgttgtgaatgagtttgtcaagcacaacctcttggccctacagcagagagtttccggattcttctccgtgaacttgacagcccgaaagccagagaagcggagccccgagactaacgcttatacagtcaaattcttggcgcagtcaccctggcaaccggactgctgcgctgtttttgcgggggccctgtactacccacggtaccggtggttcgatagggtgatgatacagttcataatgcgaatgacggggggagagaccgacgcatcgaaagaggtggagtacactgactggcagcaggtgcagcggttcgcgcgagacttcgcgcagttaccgggtaagtcctactga

SEQ ID NO: 143 - DNA, 543 bp, synthetic construct
  source 1..543: mol_type = other DNA; note = Recombinant
aagaccttgattctattctccacaagggacggccagactagggagatcgcttcctacctggccagcgagctaaaggagcttggcattcaggcagacgtggctaacgtgcaccgaattgaggagccgcagtgggagaactacgatcgggtcgtgatcggcgccagcatccggtatggacactaccacagcgcgttccaggagttcgtgaaaaagcacgcgacccgtctgaatagcatgccatcagcgttctactcggtcaacctcgtggctcgtaagcccgagaagcggacaccccagaccaactcgtatgccaggaagttccttatgaactcgcagtggcgaccggaccgctgcgcggtgatcgccggtgcgctcaggtaccctcgttataggtggtacgacaggtttatgattaaacttataatgaaaatgagcggcggagagaccgacaccagaaaagaggtggtttacacagactgggagcaggtagcaaacttcgctagggagattgctcacctcaccgacaagccgaccttgaagtaa

SEQ ID NO: 144 - DNA, 534 bp, synthetic construct
  source 1..534: mol_type = other DNA; note = Recombinant
aaggcccttatactgttcagttccagagaaggccagacgagggagatagcgagttacattgccaactcgataaaggaggaaatggaatgcgacgtgttcaacatccttcgtgtggagcagatcgactggtctcaatacgaccgcgtcctgatcgggggctcgatacactacggccatttccacccagcggtggcaaaatttgtcaagaggcacctccatgagttgcaacagaggtcttccggctttttctgcgtcaacctgacggccaggaaggccgacaagcggactccccagaccaatgcctacatgagaaagttcttgttgcagtccccatggcaacccgattgctgcgccgtgtttgcgggggcccttaggtacacccgttacaggtggttcgacagggtaatgattcagctgatcatgaggatgacgggcggagagactgacacatcgaaggaggtggagtacacagactggacgcaggtcgcccgcttcgcgcaggagttcgcccatttgcccggcaaaactcagtga

SEQ ID NO: 145 - DNA, 537 bp, synthetic construct
  source 1..537: mol_type = other DNA; note = Recombinant
aaggctcttatcgtattctcttcgagggatggccaaacccgagcgatcgcgtcttatattgctaataccctcaaagggaccctagagtgcgacgtcgtcaacgtcctcaatgctaacgacattgatttgagccagtacgaccgtgtggccattggcgcctccattcgctacgggaggttccacccagctgttaaccagtttatccggaagcaccttacgagcctccagcagctaccatctgcgttcttctccgtgaacctcacagctcggaagcccgagaagaggactatacaaaccaacgcgtacactaggaagtttctactgaactcgccgtggcagccggacctgtgctgcgtgttcgcgggagcccttcgctatccccgttacaggtggtttgaccgagtgatgattcaactcataatgcgcataacggggggcgagacagactccaccaaggagatcgagtacaccgactggcagcaggtcgcgcgattcgcccaggattttgcacagcttgccgcaaagaacccggcatga

SEQ ID NO: 146 - DNA, 537 bp, synthetic construct
  source 1..537: mol_type = other DNA; note = Recombinant
aagaccttgatcctattctccaccagggacggccaaacacacaagatcgcaaggcacatcgcaggagtcctcgaagagcaggggaaggcctgcgagttggtcgatctgttacagcccggcgaaccagactggagtaccgttgaatgcgtcgttctaggggccagcattagatatggtcacttccataagtctttcatcaggttcgtaaacactcacgcgcagcgcttgaataatatgccaggcgcccttttcacagttaacttagtcgcccgaaagcccgagaagcagagtccacagacgaactcttacacccgcaagtttctcgccgcctccccttggcagccacagcgatgccaagttttcgcgggcgctttgaggtaccctaggtactcgtggtacgacagaatgatgatacgtttgataatgaagatggccgggggcgagactgacacaaggaaggaggttgagtacactgactggcagtcggtgactcggttcgcgagggagatcgctcagctgccgggagagacgcggtag

SEQ ID NO: 147 - DNA, 540 bp, synthetic construct
  source 1..540: mol_type = other DNA; note = Recombinant
aagaccctaatactgttctctacccgcgacgggcagacaagggagatcgccgcgttccttgcctcggagctgaaggagcaggggatttacgctgacgtcataaaccttaaccggacggaggagatagcttggcaggagtatgatagagtcgtaatcggggcgtcgatccgatacgggcatttccaccctgctgtcgaccgcttcgtgaagaagcacacagagacactcaactcactgcccggcgcctttttctctgtaaaccttgttgcccggaaagccgagaagagaacgccgcagacgaactcatacaccaggaagttcctattaaacagcccgtggaagccagcggcctgcgcggtctttgctggggccctccgctaccctagataccgctggtacgacaggttcatgatacgactgattatgaaaatgacaggcggggagacggatacccgaaaggaggtagtctacactgactggtcgcaggtcgcgtcgtttgccagagagatagtccagttgaccaggtcatcgcgcttgtga

SEQ ID NO: 148 - DNA, 537 bp, synthetic construct
  source 1..537: mol_type = other DNA; note = Recombinant
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacggcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 149 - DNA, 537 bp, synthetic construct
  source 1..537: mol_type = other DNA; note = Recombinant
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgaatattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 150 - DNA, 537 bp, synthetic construct
  source 1..537: mol_type = other DNA; note = Recombinant
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatagtacaagaaggccctctag

SEQ ID NO: 151 - DNA, 534 bp, synthetic construct
  source 1..534: mol_type = other DNA; note = Recombinant
aaggccttgatcctgttctctacacgcgacggacagacacagaagatcgcatctgccatcgctgatgagataaaggggcagcaatcgtgcgacgtgattaacatacaggatgccaaaaccctcgactggcagcagtacgaccgggtactcatcggcgcctccattcgttacgggcatttccagcccgttgtgaatgagtttgtcaagcacaacctcttggccctacagcagagagtttccggattcttctccgtgaacttgacagcccgaaagccagagaagcggagccccgagactaacgcttatacagtcaaattcttggcgcagtcaccctggcaaccggactgctgcgctgtttttgcgggggccctgtactacccacggtaccggtggttcgatagggtgatgatacagttcataatgcgaatgacggggggggagaccgacgcatcgaaagaggtggagtacactgactggcagcaggtgcagcggttcgcgcgagacttcgcgcagttaccgggtaagtcctactga

SEQ ID NO: 152 - DNA, 540 bp, synthetic construct
  source 1..540: mol_type = other DNA; note = Recombinant
aagaccctaatactgttctctacccgcgacgggcagacaagggagatcgccgcgttccttgcctcggagctgaaggagcaggggatttacgctgacgtcataaaccttaaccggacggaggagatagcttggcaggagtatgatagagtcgtaatcggggcgtcgatccgatacgggcatttccaccctgctgtcgaccgcttcgtgaagaagcacacagagacactcaactcactgcccggcgcctttttctctgtaaaccttgttgcccggaaagccgagaagagaacgccgcagacgaactcatacaccaggaagttcctattaaacagcccgtggaagccagcggcctgcgcggtctttgctggggccctccgctaccctagataccgctggtacgacaggttcatgatacgactgattatgaaaatgacaggcggggagacggatacccgaaaggaggtagtctacactgactggtcgcagatcgcgtcgtttgccagagagatagtccagttgaccaggtcatcgcgcttgtga

SEQ ID NO: 153 - DNA, 540 bp, synthetic construct
  source 1..540: mol_type = other DNA; note = Recombinant
atgaaggcgctcgtgctctacagcacacgcgacggccagactcatgcgatcgcctcttacatcgcgtcctgtatgaaggagaaggccgagtgcgacgtcatcgatctcacgcacggggagcacgtgaatcttacgcagtacgaccaagtgctgataggcgcctctatccgttacggccattttaacgccgtcctcgacaaattcatcaagcgcaatgtagaccagctgaacaacatgccctccgcgttcttttgcgtgaacctgacggctcggaagcctgagaagcgaacacctcagaccaacccatacgtgcggaaattcctactcgcaacgccatggcagcccgccctgtgcggggttttcgcaggggcgctacgctatccgcgttaccgctggatcgataaggtgatgatccagctaataatgcgcatgaccggcggcgagacagacacatcgaaggaagtcgaatacacagactgggaacaggtgaagaagtttgcagaggatttcgccaagctctcatacaaaaaggcattgtga

SEQ ID NO: 154 - DNA, 537 bp, synthetic construct
  source 1..537: mol_type = other DNA; note = Recombinant
atgaaggcgcttatactgttctcgacacgcgacggtcagacgcagaaaatcgcctcagccatcgccgacgagatcaagggccagcagagctgcgatgtgatcaatattcaggacgccaaaactctcgactggcagcagtatgaccgcgtgctcattggcgcatcaatccgctacgggcatttccagccagtcgtcaatgagtttgtgaaacataacctcttggcattgcagcagcgggtgtctggcttcttctccgtgaaccttacagctagaaaaccagagaagcggtcgcccgagactaacgcctacaccgttaagttccttgcgcagtcaccgtggcagcctgattgctgcgcggtcttcgccggggcactgtactaccctcgataccggtggtttgatagggtaatgatccagttcataatgcgcatgaccggtggggagaccgacgcaagtaaagaagttgagtacacggattggcagcaggtgcaaaggttcgcacgcgacttcgcgcagctcccgggcaagtcttactga

SEQ ID NO: 155 - DNA, 534 bp, synthetic construct
  source 1..534: mol_type = other DNA; note = Recombinant
atgaaagccctgatcctctattccaccagggacggccagacccgcaagatagcctcctccatcgctgatgtcatccgccagcagcagcagtgcgacgttttaaacattaaggacgcttcactgcctgattgggcccagtatgaccgcgtcctgatcggcgcgtcgattcggtacggccacttccagcctgtggttgacaagttcgtcaagcagcacctgcatgagctgcagcagcgaactagcgggttcttcagtgtgaacctgacagctagaaagcccgaaaagagatccccagaaaccaacgcctatacgcagaaattccttgctcactcaccctggcagcctgactgttgtgccgtcttcgcgggcgccttgtactatccccgctaccgctggttcgatagggtgatgatccagctgattatgagaatgacgggaggggagaccgattcgaccaaggaggtagagtacactgactggcaacaggtgtcaactttcgcaaacgacttcgcacaactacccggtaagtcttga

SEQ ID NO: 156 - DNA, 546 bp, synthetic construct
  source 1..546: mol_type = other DNA; note = Recombinant
atgaaaaccctaatactgttctcgacccgcgacggccagacgcgtgagattgcgagctacctggcctccgagctcaaggagctggggatccaagccgatgtcgcgaacgtgcaccgcattgaggagccgcagtgggagaattacgatcgcgttgtgataggggccagcatccgctatggccactaccactcggcctttcaggagtttgtaaagaaacacgccacaagattaaactccatgcctagcgccttctactccgtcaaccttgtcgcgcgcaagccggagaagcggacacctcagacgaactcctacgcgcggaagttcctgatgaacagccagtggcggccggacagatgtgctgttattgcgggagccctgagatacccgaggtaccggtggtacgataggtttatgattaaacttattatgaagatgtctggtggggagactgacaccaggaaggaggtggtatatacagactgggagcaggtcgccaatttcgctcgggaaatcgcgcatctgacagacaagcctacactgaagtag

SEQ ID NO: 157 - DNA, 537 bp, synthetic construct
  source 1..537: mol_type = other DNA; note = Recombinant
atgaaggccctgatcctctttagctctagggagggccagacccgcgagatcgcgtcatatatcgcgaattccataaaggaggagatggagtgcgatgtgtttaacatccttagggtggagcaaatagactggtctcagtatgaccgtgtgctcataggggggagcatccactacggccactttcacccggccgtggcgaaattcgtcaagcgacacctccacgagcttcagcagcgctcctcagggttcttctgcgtcaacctgacagcaagaaaggcagataaacgcaccccgcagacgaacgcctacatgaggaagttccttctgcagtctccttggcagcccgattgctgcgcggtgttcgccggtgcactgcgctatacgcgctatagatggtttgatagagtcatgattcagctcatcatgcggatgaccggcggggaaacggatactagtaaggaggtggagtacacggactggacccaggtggcacgtttcgcccaggagtttgcacatcttcctgggaagacccaatga

SEQ ID NO: 158 - DNA, 540 bp, synthetic construct
  source 1..540: mol_type = other DNA; note = Recombinant
atgaaggcgctaattgtgttcagctccagggatggccagacgagggctatagcatcctatatcgccaataccttgaaaggaacgctcgagtgtgacgtggtcaacgtcttgaacgccaatgacattgacctttcccagtacgaccgagttgccataggcgcgtcgatccgctacgggcgatttcaccctgcagtcaaccagtttatacggaagcatttgacctcgctgcagcagctcccgtcagccttcttctctgtgaatttaaccgcgcggaagcctgagaaacggacgatccaaacaaacgcctatacccgaaagttcctcctgaacagcccatggcagccagacctgtgctgtgtcttcgccggcgcgttgcggtatccccgctacaggtggttcgatagagtgatgatccagctcatcatgaggatcaccgggggagagaccgatagtaccaaggagatcgagtacacggactggcagcaggtggctcgcttcgcccaggacttcgctcagttggccgcaaagaatccagcataa

SEQ ID NO: 159 - DNA, 540 bp, synthetic construct
  source 1..540: mol_type = other DNA; note = Recombinant
atgaagacactgatcctgttctcgactcgagatggccagactcataaaattgcgcgccacattgcgggggtcctggaggagcagggcaaagcgtgcgagctcgtggacttactccagcccggggagccggactggagcacggtggagtgcgtcgttctgggcgcttccatacgttacgggcatttccacaaaagtttcatccggttcgtcaacacccacgctcaacggctgaacaacatgcctggcgcgctattcactgttaacttagtggctcgtaagcccgagaagcagtctccgcagactaactcctacacaaggaaatttctagcagcaagcccatggcaaccgcagcggtgccaggtgttcgctggagctctgcgctatcctaggtacagttggtacgacagaatgatgatacggttgattatgaagatggcaggcggggagacggacaccaggaaagaggtcgaatacactgactggcaatcagtcactcggtttgctagagagatcgcgcaattaccaggtgagacgcggtaa

SEQ ID NO: 160 - DNA, 537 bp, synthetic construct
  source 1..537: mol_type = other DNA; note = Recombinant
atgaaggctctcatactgttcagctcgagagacgggcagacccagctgatcgcctcctccatagcaaaggagctagagggcaagcaagcctgcgacgtgctcaatattctcgacacaaccaacgtggagtggactcagtacgacagagtcctaatcggcgcgtccatcagatacggccacttccatcccgccgtcgctgaattcgtgaaacgccaccagcgtgagctccagcagcgcagcagcggcttcttcagcgtgaatcttactgcgagaaagccggaaaagcggagtcccgagactaacgcttatacggcaaagttcctcaaccaatctccctggcaaccagactgctgtgccgtgttcgctggggcactgaggtatccgcgctatcggtggttcgatagaatcatgatacagctgataatgcgtatgactggtggggagacggattccagtaaagaggtagagtatactgattggcagcaggtcactaggttcgcgcaggagtttgctaggctgccgggcaagacatcctga

SEQ ID NO: 161 - DNA, 543 bp, synthetic construct
  source 1..543: mol_type = other DNA; note = Recombinant
atgaaaaccttaatcttgttcagcacccgcgacggccagacgcgtgaaatcgcagcgttcctcgcttcggagctcaaggaacagggaatttacgccgacgtcattaacctaaaccgtaccgaagagattgcgtggcaggagtatgaccgcgtggtgattggcgcttctatccgctatggccacttccacccggctgttgaccggttcgtgaagaagcacacggagaccttgaactcactgccgggggcattctttagcgtaaatctggtggcgcgcaaggccgagaagcgcaccccccagacgaacagctacacccgcaaatttttacttaactccccatggaaacctgcggcctgcgcagtgttcgcaggagctctccgctatcctcgctatcgatggtacgatcggttcatgattcggctgattatgaaaatgacgggcggcgagacggatacgcgaaaggaagttgtctacactgactggtcccaggtggcctcgtttgcaagggagatcgtacagctcactcgatctagtaggctctga

SEQ ID NO: 162 - DNA, 534 bp, synthetic construct
  source 1..534: mol_type = other DNA; note = Recombinant
atgaagattctcatcttattttccacccgagacggccaaacccgcgagattgcggcgtccctcgcctccgagttgaaggagcaggcgtttgatgtggatgtggtcaacctccaccgcgcagaaaacatagcgtgggaggagtacgatggggtcgtcatcggagcgtcaatccgctacggacatttccactcaacgctgaattcatttgtgaagaagcaccaacaagcgctcaagaagctgcccggagcattctacagcgtcaacctcgtggctcggaagccggaaaagcgcaccccgcaaacaaacagctacacacgcaagtttctgctcgactcgccctggcaacccgacctgagtgccgttttcgccggggcactgcgctatccccgttacaactggtacgatcgcataatgattcgactgatcatgaagattacaggcggggaaaccgatactcggaaggaggtggtgtatacagactggcagcaggttacccacttcgcccacgagatcgtccagctcgttcgtaagtga

SEQ ID NO: 163 - protein, 442 aa, Xanthomonas campestris
MQTQPVIIAGAGIAGLSIAYELQQKGIPYEIMEASSYAGGVVKSLHIDGYELDAGPNSLAASAAFMAYIDQLGLQDQVLEAAAASKNRFLVRNDKLHAVSPHPFKILQSAYISGGAKWRLFTERFRKAAAPEGEETVSSFVTRRFGKEINDYLFEPVLSGIYAGNPDLMSVGEVLPMLPQWEQKYGSVTQGLLKNKGAMGGRKIIAFKGGNATLTNRLQSLLSGKIRFNCAVTGVTRGADDYIVQYTENGNTAMLNASRVIFTTPAYSTAVAIQALDASLATHLSDVPYPRMGVLHLGFGAEARQKAPAGFGFLVPHAAGKHFLGAICNSAIFPSRVPTGKVLFTVFLGGARQEQLFDQLGPEKLQQTVVKELMELLGLTTPPEMQRFSEWNRAIPQLNVGYAQTRQQIGVFEQRYPGIRLAGNYVTGVAVPAIIQAAKGYC

SEQ ID NO: 164 - protein, 439 aa, Xanthomonas campestris
QPVIIAGAGIAGLSIAYELQQKGIPYEIMEASSYAGGVVKSLHIDGYELDAGPNSLAASAAFMAYIDQLGLQDQVLEAAAASKNRFLVRNDKLHAVSPHPFKILQSAYISGGAKWRLFTERFRKAAAPEGEETVSSFVTRRFGKEINDYLFEPVLSGIYAGNPDLMSVGEVLPMLPQWEQKYGSVTQGLLKNKGAMGGRKIIAFKGGNATLTNRLQSLLSGKIRFNCAVTGVTRGADDYIVQYTENGNTAMLNASRVIFTTPAYSTAVAIQALDASLATHLSDVPYPRMGVLHLGFGAEARQKAPAGFGFLVPHAAGKHFLGAICNSAIFPSRVPTGKVLFTVFLGGARQEQLFDQLGPEKLQQTVVKELMELLGLTTPPEMQRFSEWNRAIPQLNVGYAQTRQQIGVFEQRYPGIRLAGNYVTGVAVPAIIQAAKGYC

SEQ ID NO: 165 - protein, 448 aa, Chitinophaga pinensis
MSDQPVLIVGAGLSGLSIAYELQKLQVPYQVLEVSGHSGGVMKSLRKDGFELDAGANTIAASPEILAYFTSLGLENEILQATAASKHRFLVRRRQLHAVSPHPFKIMSSPYLSRGSKWRLFTERFRKPVVASGEETVTDFITRRFNREIAEYVFDPVLSGIYAGNPDQMSIAEVLPALPRWEREYGSVTKGLMKDKGAMGGRKIISFKGGNQLLTNRLQQLLTTPVRFNCKVTGITASNGGYIVSAVEDGVSESYTASRVILTTPAYSAAATITNLDAATAALLNEIHYPRMGVLHLGFDATALPQPLDGFGFLVPNAENMHFLGAICNAAIFPDKAPPGKILFTVFLGGARQESLFDQMTPEALQQQVVSEVMSLLHLSAPPVMQHFSSWNKAIPQLNVGHVKLRRAVEAFEKKYPGIHLSGNYLQGVAIPALLQHAAALAASLKKN

SEQ ID NO: 166 - protein, 445 aa, synthetic construct
  source 1..445: note = Recombinant
QPVLIVGAGLSGLSIAYELQKLQVPYQVLEVSGHSGGVMKSLRKDGFELDAGANTIATSPEILAYFTSLGLENEILQATATSKHRFLVRRRQLHAVSPHPFKIMSSPYLCRGSKWRLFTERFRKPVVASGEETVTDFITRRFNREIAEYVFDPVLSGIYAGNPDQMSIAEVLPALPRWEREYGSVTKGLMKDKGAMGGRKIISFKGGNQLLTNRLQQLLTTPVRFNCKVTGITASNGGYIVSAVEDGVSESYTASRVILTTPAYSAAATITNLDAATAALLNEIHYPRMGVLHLGFDATALPQPLDGFGFLVPNAENMHFLGAICNAAIFPDKAPPGKILFTVFLGGARQESLFDQMTPEALQQQVVSEVMSLLHLSAPPVMQHFSSWNKAIPQLNVGHVKLRRAVEAFEKKYPGIHLSGNYLQGVAIPALLQHAAALAASLKKN

SEQ ID NO: 167 - protein, 470 aa, Bacillus subtilis
MSDGKKHVVIIGGGITGLAAAFYMEKEIKEKNLPLELTLVEASPRVGGKIQTVKKDGYIIERGPDSFLERKKSAPQLVKDLGLEHLLVNNATGQSYVLVNRTLHPMPKGAVMGIPTKIAPFVSTGLFSLSGKARAAMDFILPASKTKDDQSLGEFFRRRVGDEVVENLIEPLLSGIYAGDIDKLSLMSTFPQFYQTEQKHRSLILGMKKTRPQGSGQQLTAKKQGQFQTLSTGLQTLVEEIEKQLKLTKVYKGTKVTKLSHSGSGYSLELDNGVTLDADSVIVTAPHKAAAGMLSELPAISHLKNMHSTSVANVALGFPEGSVQMEHEGTGFVISRNSDFAITACTWTNKKWPHAAPEGKTLLRAYVGKAGDESIVDLSDNDIINIVLEDLKKVMNINGEPEMTCVTRWHESMPQYHVGHKQRIKELREALASAYPGVYMTGASFEGVGIPDCIDQGKAAVSDALTYLFS

SEQ ID NO: 168 - protein, 470 aa, Bacillus pumilus
MHDNQKHLVIIGGGITGLAAAFYLEKEVEEKGLPIQISLIEASPRLGGKIQTLYKDGYIIERGPDSFLERKVSGPQLAKDVGLSDQLVNNETGQAYVLVNEKLHPMPKGAVMGIPTQISPFITTGLFSVAGKARAAMDFVLPKSKQTEDQSLGEFFRRRVGDEVVENLIEPLLSGIYAGDIDRLSLMSTFPQFYQTEQQHRSLILGMKKSQQHAKAQQVTAKKQGQFQTINQGLQSLVEAVEGKLKLTTVYKGTKVKQIEKTDGGYGLQLDSGQTLFADSAIVTTPHQSIYSMFPKEAGLEYLHDMTSTSVATVALGFKDEDVHNEYDGTGFVISRNSDFSITACTWTNKKWPHTAPKGKTLLRAYVGKAGDESIVEQSDSQIVSIVLEDLKKIMDIKADPELTTVTRWKTSMPQYHVGHQKAISNMRETFKQSYPGVYITGAAFEGVGIPDCIDQGKAAISEAVSYLFS

SEQ ID NO: 169 - protein, 470 aa, Bacillus pumilus
MHDNQKHLVIIGGGITGLAAAFYLEKEVEEKGLPIQISLIEASPRLGGKIQTLYKDGYIIERGPDSFLERKVSGPQLAKDVGLSDQLVNNETGQAYVLVNETLHPMPKGAVMGIPTQISPFITTGLFSVAGKARAAMDFVLPKSKQTEDQSLGEFFRRRVGDEVVENLIEPLLSGIYAGDIDRLSLMSTFPQFYQTEQKHRSLILGMKKSQQHAKAQQVTAKKQGQFQTINQGLQALVEAVESKLKLTTIYKGTKVKQIEKTDGGYGVQLDSGQTLLADSAIVTTPHQSIYSMFPKEAGLEYLHDMTSTSVATVALGFKEEDVHNEYDGTGFVISRNSDFSITACTWTNKKWPHTAPKGKTLLRAYVGKAGDESIVEQSDHQIVSIVLEDLKKIMDIKADPELTTVTRWKTSMPQYHVGHQKAISNMRETFKQSYPGVYITGAAFEGVGIPDCIDQGKAAISEAVSYLFS

SEQ ID NO: 170 - protein, 477 aa, Paenibacillus macerans
MSKKIAVIGGGITGLSVAYYVRKLLREQGVNAGVTLVEQSDRLGGKIRSLRRDGFTIEQGPDSMIARKPAALELIRELGLEDKLAGTNPQAKRSYILHRGKFHPMPPGLMLGIPTQMWPMVKTGLLSPAGKLRAAMDLLLPARRGGGDESLGGFIRRRLGREVLEQMTEPLLAGIYAGDTEQLSLKATFPQFMEMERKHRSLILGLLAGKKQPPRPGGSQVPLPKAAQTSMFLTLTGGLEGLTEALEESLSEEKIITGQAVTGLSQQEAGYELNLSGGERLNADGVILAVPAFAAARLLDGVPEAAYLERIRYVSVANLAFAYRREDVPHDLNGSGVLIPRGEGRMITAITWVSSKWLHSAPGDKALLRAYIGRLGDEAWTAMCRADIERRVAAELRDLLGIAASPLFCELAALPESMPQYPVGHVERLEALRGALCRAKPGLLLCGAGYAGVGIPDCIRQGKEAAESMAAYLRDGR

SEQ ID NO: 171 - protein, 493 aa, Paenibacillus thiaminolyticus
MKALRKLVVIGGGITGLSAAFYALKQADEEGQPISVTIIEQSDRLGGKIQTLRKEGCVIEKGPDSFLARKLPMIDLARDLGMDSELVATNPHAKKTYILRRGKLYRMPPGLVLGIPTELGPFAKTGLISPWGKLRAAMDLFIKPHPADEDESVGAFLDRRLGREVTEHIAEPLLAGIYAGDLQALSLQATFPQFAQVERKHGGLIRGMKASRQAGQSVPGLPDVAKGTMFLTFRNGLTSLVERLEETLRDRAELCLGIGAEGFEKREDGTYLVRLSDGSRLQADAVIVTTPSYHAASLLEEHVDASALQAIRHVSVANVVSVFDRKQVNNQFDGTGFVISRREGRAITACTWTSVKWPHTSRGDKLIIRCYIGRAGDEERVDWPDEALKRTVRSELRELLDIDIDPEFVEITRLRHSMPQYPVGHVQAIRSLRDEVGRTLPGVFLAGQPYEGVGMPDCVRSGRDAAEAAVSAMQAMSTEPEAPAEDAATGTAG

SEQ ID NO: 172 - protein, 474 aa, Paenibacillus polymyxa
MGDKKRRVVVVGGGLTGLSAAFYIRKHYREAGVEPVITLVEKSSSMGGMIETLHRDGFVIEKGPDSFLARKTAMIDLAKELEIDHELVSQNPESKKTYIMQRGKLHPMPAGLVLGIPTELRPFLRSGLVSPAGKLRALMDFVIPPRRTTEDESLGYMIERRLGAEVLENLTEPLLAGIYAGDMRRLSLQATFPQFGEVERDYGSLIRGMMTGRKPAETHTGTKRSAFLNFRQGLQSLVHALVHELQDVDQRLNTAVKSLQRLDGAQTRYRVELGNGEMLEADDVVVTVPTYVASELLKPHVDTAALDAINYVSVANVVLAFEKKEVEHVFDGSGFLVPRKEGRNITACTWTSTKWLHTSPDDKVLLRCYVGRSGDEQNVELPDEALTNLVLKDLRETMGIEAVPIFSEITRLRKSMPQYPVGHLQHIAALREELGSKLPGVYIAGAGYEGVGLPDCIRQAKEMSVQATQELAAD

SEQ ID NO: 173 - protein, 470 aa, Bacillus atrophaeus
MSDGKKHLVIIGGGITGLASAFYMEKEIREKNLPLSVTLVEASPRVGGKIQTARKDGYIIERGPDSFLERKKSAPELVEDLGLEHLLVNNATGQSYVLVNETLHPMPKGAVMGIPTKIAPFMSTGLFSFSGKARAAMDFVLPASKPKEDQSLGEFFRRRVGDEVVENLIEPLLSGIYAGDIDRLSLMSTFPQFYQTEQKHRSLILGMKKTRPQGSGQRLTAKKQGQFQTLKTGLQTLVEELENQLKLTKVYKGTKVTNISRGEKGCSIALDNGMTLDADAAIVTSPHKSAAGMFPDLPAVSQLKDMHSTSVANVALGFPQEAVQMEHEGTGFVISRNSDFSITACTWTNKKWPHSAPEGKTLLRAYVGKAGDESIVELSDNEIIKIVLEDLKKVMKIKGEPEMTCVTRWNESMPQYHVGHKQRIKKVREALAASYPGVYMTGASFEGVGIPDCIDQGKSAVSDVLAYLFG

SEQ ID NO: 174 - protein, 470 aa, Bacillus atrophaeus
MSDGKKHLVIIGGGITGLASAFYMEKEIREKNLPLSVTLVEASPRVGGKIQTARKDGYIIERGPDSFLERKKSAPELVEDLGLEHLLVNNATGQSYVLVNETLHPMPKGAVMGIPTKIAPFMSTRLFSFSGKARAAMDFVLPASKPKEDQSLGEFFRRRVGDEVVENLIEPLLSGIYAGDIDRLSLMSTFPQFYQTEQKHRSLILGMKKTRPQGSGQQLTAKKQGQFQTLKTGLQTLVEELENQLKLTKVYKGTKVTNISRGEKGCSIALDNGMTLDADAAIVTSPHKSAAGMFPDLPAVSQLKDMHSTSVANVALGFPQEAVQMEHEGTGFVISRNSDFSITACTWTNKKWPHSAPEGKTLLRAYVGKAGDESIVELSDNEIIKIVLEDLKKVMKIKGEPEMTCVTRWNESMPQYHVGHKQRIKKVREALAASYPGVYMTGASFEGVGIPDCIDQGKSAVSDVLAYLFE

SEQ ID NO: 175 - protein, 475 aa, synthetic construct
  source 1..475: note = Recombinant
KKIAVIGGGITGLSVAYYVRKLLREQGVNAGVTLVEQSDRLGGKIRSLRRDGFTIEQGPDSMIARKPAALELIRELGLEDKLAGTNPQAKRSYILHRGKFHPMPPGLMLGIPTQMWPMVKTGLLSPAGKLRAAMDLLLPARRGGGDESLGGFIRRRLGREVLEQMTEPLLAGIYAGDTEQLSLKATFPQFMEMERKHRSLILGLLAGKKQPPRPGGSQVPLPKAAQTSMFLTLTGGLEGLTEALEESLSEEKIITGQAVTGLSQQEAGYELNLSGGERLNADGVILAVPAFAAARLLDGVPEAAYLERIRYVSVANLAFAYRREDVPHDLNGSGVLIPRGEGRMITAITWVSSKWLHSAPGDKALLRAYIGRLGDEAWTAMCRADIERRVAAELRDLLGIAASPLFCELAALPESMPQYPVGHVERLEALRGALCRAKPGLLLCGAGYAGVGIPDCIRQGKEAAESMAAYLRDGR

SEQ ID NO: 176 - protein, 489 aa, synthetic construct
  source 1..489: note = Recombinant
RKLVVIGGGITGLSAAFYALKQADEEGQPISVTIIEQSDRLGGKIQTLRKEGCVIEKGPDSFLARKLPMIDLARDLGMDSELVATNPHAKKTYILRRGKLYRMPPGLVLGIPTELGPFAKTGLISPWGKLRAAMDLFIKPHPADEDESVGAFLDRRLGREVTEHIAEPLLAGIYAGDLQALSLQATFPQFAQVERKHGGLIRGMKASRQAGQSVPGLPDVAKGTMFLTFRNGLTSLVERLEETLRDRAELCLGIGAEGFEKREDGTYLVRLSDGSRLQADAVIVTTPSYHAASLLEEHVDASALQAIRHVSVANVVSVFDRKQVNNQFDGTGFVISRREGRAITACTWTSVKWPHTSRGDKLIIRCYIGRAGDEERVDWPDEALKRTVRSELRELLDIDIDPEFVEITRLRHSMPQYPVGHVQAIRSLRDEVGRTLPGVFLAGQPYEGVGMPDCVRSGRDAAEAAVSAMQAMSTEPEAPAEDAATGTAG

SEQ ID NO: 177 - protein, 469 aa, synthetic construct
  source 1..469: note = Recombinant
RRVVVVGGGLTGLSAAFYIRKHYREAGVEPVITLVEKSSSMGGMIETLHRDGFVIEKGPDSFLARKTAMIDLAKELEIDHELVSQNPESKKTYIMQRGKLHPMPAGLVLGIPTELRPFLRSGLVSPAGKLRALMDFVIPPRRTTEDESLGYMIERRLGAEVLENLTEPLLAGIYAGDMRRLSLQATFPQFGEVERDYGSLIRGMMTGRKPAETHTGTKRSAFLNFRQGLQSLVHALVHELQDVDQRLNTAVKSLQRLDGAQTRYRVELGNGEMLEADDVVVTVPTYVASELLKPHVDTAALDAINYVSVANVVLAFEKKEVEHVFDGSGFLVPRKEGRNITACTWTSTKWLHTSPDDKVLLRCYVGRSGDEQNVELPDEALTNLVLKDLRETMGIEAVPIFSEITRLRKSMPQYPVGHLQHIAALREELGSKLPGVYIAGAGYEGVGLPDCIRQAKEMSVQATQELAAD

SEQ ID NO: 178 - protein, 465 aa, synthetic construct
  source 1..465: note = Recombinant
KHLVIIGGGITGLASAFYMEKEIREKNLPLSVTLVEASPRVGGKIQTARKDGYIIERGPDSFLERKKSAPELVEDLGLEHLLVNNATGQSYVLVNETLHPMPKGAVMGIPTKIAPFMSTRLFSFSGKARAAMDFVLPASKPKEDQSLGEFFRRRVGDEVVENLIEPLLSGIYAGDIDRLSLMSTFPQFYQTEQKHRSLILGMKKTRPQGSGQQLTAKKQGQFQTLKTGLQTLVEELENQLKLTKVYKGTKVTNISRGEKGCSIALDNGMTLDADAAIVTSPHKSAAGMFPDLPAVSQLKDMHSTSVANVALGFPQEAVQMEHEGTGFVISRNSDFSITACTWTNKKWPHSAPEGKTLLRAYVGKAGDESIVELSDNEIIKIVLEDLKKVMKIKGEPEMTCVTRWNESMPQYHVGHKQRIKKVREALAASYPGVYMTGASFEGVGIPDCIDQGKSAVSDVLAYLFE

SEQ ID NO: 179 - protein, 473 aa, synthetic construct
  source 1..473: note = Recombinant
IAVIGGGITGLSVAYYVRKLLREQGVNAGVTLVEQSDRLGGKIRSLRRDGFTIEQGPDSMIARKPAALELIRELGLEDKLAGTNPQAKRSYILHRGKFHPMPPGLMLGIPTQMWPMVKTGLLSPAGKLRAAMDLLLPARRGGGDESLGGFIRRRLGREVLEQMTEPLLAGIYAGDTEQLSLKATFPQFMEMERKHRSLILGLLAGKKQPPRPGGSQVPLPKAAQTSMFLTLTGGLEGLTEALEESLSEEKIITGQAVTGLSQQEAGYELNLSGGERLNADGVILAVPAFAAARLLDGVPEAAYLERIRYVSVANLAFAYRREDVPHDLNGSGVLIPRGEGRMITAITWVSSKWLHSAPGDKALLRAYIGRLGDEAWTAMCRADIERRVAAELRDLLGIAASPLFCELAALPESMPQYPVGHVERLEALRGALCRAKPGLLLCGAGYAGVGIPDCIRQGKEAAESMAAYLRDGR

SEQ ID NO: 180 - protein, 487 aa, synthetic construct
  source 1..487: note = Recombinant
LVVIGGGITGLSAAFYALKQADEEGQPISVTIIEQSDRLGGKIQTLRKEGCVIEKGPDSFLARKLPMIDLARDLGMDSELVATNPHAKKTYILRRGKLYRMPPGLVLGIPTELGPFAKTGLISPWGKLRAAMDLFIKPHPADEDESVGAFLDRRLGREVTEHIAEPLLAGIYAGDLQALSLQATFPQFAQVERKHGGLIRGMKASRQAGQSVPGLPDVAKGTMFLTFRNGLTSLVERLEETLRDRAELCLGIGAEGFEKREDGTYLVRLSDGSRLQADAVIVTTPSYHAASLLEEHVDASALQAIRHVSVANVVSVFDRKQVNNQFDGTGFVISRREGRAITACTWTSVKWPHTSRGDKLIIRCYIGRAGDEERVDWPDEALKRTVRSELRELLDIDIDPEFVEITRLRHSMPQYPVGHVQAIRSLRDEVGRTLPGVFLAGQPYEGVGMPDCVRSGRDAAEAAVSAMQAMSTEPEAPAEDAATGTAG

SEQ ID NO: 181 - protein, 467 aa, synthetic construct
  source 1..467: note = Recombinant
VVVVGGGLTGLSAAFYIRKHYREAGVEPVITLVEKSSSMGGMIETLHRDGFVIEKGPDSFLARKTAMIDLAKELEIDHELVSQNPESKKTYIMQRGKLHPMPAGLVLGIPTELRPFLRSGLVSPAGKLRALMDFVIPPRRTTEDESLGYMIERRLGAEVLENLTEPLLAGIYAGDMRRLSLQATFPQFGEVERDYGSLIRGMMTGRKPAETHTGTKRSAFLNFRQGLQSLVHALVHELQDVDQRLNTAVKSLQRLDGAQTRYRVELGNGEMLEADDVVVTVPTYVASELLKPHVDTAALDAINYVSVANVVLAFEKKEVEHVFDGSGFLVPRKEGRNITACTWTSTKWLHTSPDDKVLLRCYVGRSGDEQNVELPDEALTNLVLKDLRETMGIEAVPIFSEITRLRKSMPQYPVGHLQHIAALREELGSKLPGVYIAGAGYEGVGLPDCIRQAKEMSVQATQELAAD

SEQ ID NO: 182 - protein, 463 aa, synthetic construct
  source 1..463: note = Recombinant
LVIIGGGITGLASAFYMEKEIREKNLPLSVTLVEASPRVGGKIQTARKDGYIIERGPDSFLERKKSAPELVEDLGLEHLLVNNATGQSYVLVNETLHPMPKGAVMGIPTKIAPFMSTRLFSFSGKARAAMDFVLPASKPKEDQSLGEFFRRRVGDEVVENLIEPLLSGIYAGDIDRLSLMSTFPQFYQTEQKHRSLILGMKKTRPQGSGQQLTAKKQGQFQTLKTGLQTLVEELENQLKLTKVYKGTKVTNISRGEKGCSIALDNGMTLDADAAIVTSPHKSAAGMFPDLPAVSQLKDMHSTSVANVALGFPQEAVQMEHEGTGFVISRNSDFSITACTWTNKKWPHSAPEGKTLLRAYVGKAGDESIVELSDNEIIKIVLEDLKKVMKIKGEPEMTCVTRWNESMPQYHVGHKQRIKKVREALAASYPGVYMTGASFEGVGIPDCIDQGKSAVSDVLAYLFE

SEQ ID NO: 183 - DNA, 1329 bp, Xanthomonas campestris
  source 1..1329: mol_type = genomic DNA
atgcaaacacagcccgttatcattgccggcgccggtattgccggactaagtatagcttacgaattacagcagaaaggcattccctatgaaatcatggaggcctcttcctatgcaggaggcgttgtgaaatcattacatattgatggttatgaactggatgctggccctaattcgctggccgcatctgcagcattcatggcttatatcgatcaactgggtttgcaggaccaggtattggaagctgcggctgccagtaagaaccgctttctggtcagaaatgataaattgcatgcagtatcgccacatccctttaagatactgcagtcagcatatatcagtggtggcgccaagtggcgtctgttcacagaaagatttcgaaaagcggccgctccggagggagaggaaacagtatcttcctttgtgacccgccgttttggaaaggagatcaatgactacctttttgaacccgtgctttctggtatatatgcaggtaatcctgatctgatgtcagttggtgaagtactgcctatgctgccacaatgggagcaaaaatacggtagtgttacgcagggactcctgaagaataaaggagctatgggtggacgtaagatcattgcctttaaaggaggtaatgcgacactgacaaacagattgcaatccctgcttagcggtaagataagatttaactgtgccgtaacgggtgtaacccgtggggcggacgactatattgtacaatataccgagaatggtaatacagctatgctgaatgcatcccgtgtgatattcaccacccctgcatacagtacagccgtagctatacaggcacttgacgcttcccttgctacacatctcagcgatgttccctatccccgtatgggcgtactgcacctggggtttggagcggaagcccggcagaaagcaccggcaggttttggtttcctggtgccgcatgctgcaggaaagcatttcctgggcgctatctgtaacagcgctatattcccttcccgcgtaccgacaggtaaagtgctgtttacggtgttcctgggtggcgcgagacaagaacagctgtttgatcagctggggcctgaaaagctacagcagacagtagtgaaagaactgatggaactgctgggcctgactacaccaccagaaatgcagcgttttagtgaatggaacagagcgattccgcaactaaatgtaggttatgcacagacgaggcagcagataggcgtttttgaacagcgttacccgggcatcagattagcgggtaactatgtgaccggagtggctgtacccgctatcatacaggccgcaaaagggtactgttga

SEQ ID NO: 184 - DNA, 1347 bp, Chitinophaga pinensis
  source 1..1347: mol_type = genomic DNA
atgtctgatcaacccgtattgattgtcggcgccggcttatccggattgagcattgcgtatgaattgcagaaactgcaggtgccttaccaggtactggaagtttcgggtcatagcggcggcgtgatgaaatcattacggaaagatggatttgaactggatgcaggcgctaatacaatcgcagcttctcctgaaatactggcatacttcacatcactgggactggaaaatgagatattgcaggccaccgctgccagcaagcaccggttcctggtaagacggcggcagttgcacgctgtttctccccatcctttcaagatcatgtcgtctccttacctgagcaggggcagtaaatggcggttgtttaccgaacgttttcgcaaacctgttgtggcaagcggagaagaaaccgtcaccgattttataacaagaaggtttaaccgggagatagcagaatatgtgtttgacccggtattatccggcatatatgccggcaatcccgaccagatgagcatagcggaagtattacctgcgttgccgcgctgggagcgggaatatgggagtgttaccaaagggctgatgaaagataaaggcgcaatgggcggccggaagattatcagttttaaaggtggtaaccagttgctcacaaaccgtttgcagcaattgctcactaccccggtgcgctttaattgtaaggtaaccggtatcaccgcatccaatggcggctatattgtaagcgctgtagaagatggcgtatcagaaagttatactgcttcaagggtgatattaaccacacctgcttacagcgcggcagcaactattacgaatcttgatgctgctaccgctgccttgttaaatgaaattcattatccccgtatgggcgtgctgcacctgggttttgacgctactgcgttgccgcagcccctggatggatttggtttcctggtaccgaatgctgaaaatatgcatttcctgggagcaatctgcaacgctgcaattttcccggataaggcgcctccgggaaaaatcctctttacggtattcctgggaggagcaagacaggaaagtttgtttgaccagatgacgcccgaagctctgcaacagcaggtagtttcagaggtcatgtctttactgcatttatctgcgccgccggtaatgcagcatttcagtagctggaataaagcgattccgcagttaaatgtgggtcatgttaagttacggcgtgccgtggaagcttttgaaaaaaaatatcccggtattcacctcagcgggaattacctgcaaggcgtagctatcccggctttactgcaacatgccgccgctttggcggcttccctgaagaaaaattaa

SEQ ID NO: 185 - DNA, 1335 bp, synthetic construct
  source 1..1335: mol_type = other DNA; note = Recombinant
caacccgtcctcatcgttggagctggtctctccgggctctcaatcgcttacgaactacagaagctgcaagtcccttaccaagtgctggaggtttctggacattctggtggagtcatgaagtcactccggaaggacggatttgaactcgacgctggtgccaacaccatagccacgtctcccgagattcttgcgtactttacctcactaggtcttgagaatgagatcctccaggcgactgctacttctaaacaccgcttcttggtgcggcgaaggcaactgcacgccgtgagcccgcacccgttcaagatcatgtcatcgccgtacctctgccgtggctccaaatggaggctctttactgagcggtttcggaaacccgtcgtcgcttcgggcgaggagaccgtcaccgatttcatcacgaggagattcaaccgcgaaatagcggagtatgtgttcgaccctgttctaagtgggatctacgccgggaacccggaccaaatgagtattgctgaggtgttgcctgccttgcctaggtgggaaagggagtacggatcagtgaccaagggccttatgaaggataagggtgcgatgggaggtcgaaagatcatcagctttaagggtggcaaccagctacttacaaaccgcttacagcagctactcactactccggtgagattcaattgcaaggtgacagggattacagccagcaatggcgggtacatcgtgagcgctgttgaggacggcgtatctgagagctacaccgcatctcgtgtgatcttgaccacacccgcttactcagcagcggctaccataactaaccttgatgcagccactgcggcactgttgaacgaaatccattatccacgtatgggcgtgttacacttgggctttgatgcaactgccttgccacagccgctggacgggttcggatttctagtgccgaacgcggagaacatgcacttcctgggagccatctgcaatgcagccatcttcccggacaaggctccgcccggcaagatcctgtttacagtgttcctcggaggcgcacgccaggagtcgctcttcgatcagatgactcctgaggctcttcagcagcaagtcgttagtgaggtgatgagcttgttgcacttgtcagctccaccggtgatgcagcacttctcctcctggaacaaggccatccctcaattgaacgtcgggcacgtgaagttgcggcgcgcggtagaggcgttcgagaagaaataccctggaatccatctctcgggcaactacctccagggagttgcaataccagctttactccagcacgccgcagctttagctgcttctcttaagaagaac

SEQ ID NO: 186 - DNA, 1413 bp, Bacillus subtilis
  source 1..1413: mol_type = genomic DNA
atgagtgacggcaaaaaacatgtagtcatcatcggcggcggcattaccggtttagccgccgccttctatatggaaaaagaaatcaaagaaaagaatctgccgcttgaactgacgcttgttgaggcaagtccgagagtcggcggaaaaatccagactgtcaagaaggacggctatatcatcgaaagagggccagactcatttctggaacgaaagaaaagcgccccgcagcttgttaaagacttaggtcttgagcatttgcttgtcaacaatgcgaccggacaatcctatgtgcttgtaaaccgcactctgcatccaatgccgaagggcgctgtaatggggataccgacaaaaattgcgccgtttgtttctacgggtctgttttctttgtctgggaaggcgagagctgctatggatttcatcctgcctgctagcaaaacaaaggatgatcagtcattgggagaattcttccgcagacgtgtcggagatgaagtggtcgagaacttaatcgagccgcttctatcagggatctacgcaggcgacattgacaagctcagcctgatgtcgacattcccgcaattttatcagacggaacaaaagcatagaagcctgattctcggcatgaaaaaaacaaggcctcaaggctcaggccagcagctgacggcaaaaaaacaagggcagttccagactctgtcaaccggtttgcagacccttgtagaagagatcgaaaagcagttaaagctgacgaaggtgtataaaggcacaaaagtgaccaaactcagccatagcggctctggctattcgctcgaactggataacggcgtcacacttgatgctgattcagtaattgtgactgctccgcataaagcggctgcgggaatgctttctgagcttcctgccatttctcatttgaaaaatatgcactccacatccgtggcaaacgtcgctttaggtttccctgaaggctccgtccaaatggagcatgagggcacgggttttgtcatttcaagaaacagtgactttgcgatcacagcctgtacgtggacgaataaaaaatggccgcacgcagcgccggaaggcaaaacgctgcttcgggcatatgtcggaaaagctggagacgaatccattgtcgatctatcagataatgacattatcaacattgtgttagaagacttaaagaaagtcatgaacataaacggcgagccggaaatgacatgtgtcacccgatggcatgaaagcatgccgcagtaccatgtcggccataagcagcgtatcaaggagctgcgtgaagcacttgcatctgcgtatccgggtgtttatatgacaggcgcttctttcgaaggtgtcggcattcccgactgcattgatcaaggaaaagctgccgtgtctgacgcgcttacctatttattcagctaa

SEQ ID NO: 187 - DNA, 1413 bp, Bacillus pumilus
  source 1..1413: mol_type = genomic DNA
atgcatgacaatcaaaaacaccttgtcatcattggcggtggcatcactggtttagccgccgccttctatttggaaaaggaagtcgaggaaaaaggtcttccgattcaaatatcacttattgaagcgagccctaggctaggtggaaaaatacaaacattatataaagacggctacatcattgaacgtggacctgattcatttttagaaagaaaggtcagtgggccgcagcttgcaaaagatgtcggtctgtccgatcagctcgtcaataatgaaactgggcaagcgtatgtactggtcaatgaaaagcttcacccgatgccaaaaggtgctgttatggggattccaactcaaatcagcccatttattacaactggtcttttttcagttgcgggaaaagcaagagcggcgatggatttcgtgttgccaaaaagcaagcagacggaagaccagtcgcttggtgaattttttagaagacgtgtgggtgatgaggtcgttgagaatttaattgagccgcttctatcaggcatttatgcaggggatattgaccgtctgagcttaatgtcgaccttcccgcaattttatcaaacagaacagcagcatcgaagtttgattcttgggatgaaaaaatcacagcagcatgcgaaagcgcagcaagtgactgcgaaaaaacaaggacagttccaaacgatcaatcaaggattgcagtcgcttgtggaagcagtagaaggtaagctcaagctgacaacggtctataaagggacaaaagtcaaacaaattgaaaaaacggatggaggctatggcttacaattagacagcggtcaaacgctttttgccgattcagccattgtcacgactccgcatcaatcgatttattccatgtttcctaaagaagcagggctagagtatttgcatgacatgacctctacttctgttgcaacagtagcactcggttttaaagatgaggatgttcataatgaatatgacggcactggatttgtcatctcaagaaacagtgatttctctattacggcctgtacatggacaaacaaaaaatggccgcatactgctccgaaaggaaaaacgctattgcgtgcgtatgtagggaaggctggcgacgaatcaattgtcgagcagtcagacagtcaaatcgtcagcattgtgctagaagatttaaagaaaatcatggatattaaagcagatccagaattgacgacagtgactcgctggaagacaagtatgccgcaatatcacgtcggtcatcagaaagccatttcgaacatgcgagaaacgtttaagcaatcatatcctggtgtttatattacaggtgctgcttttgaaggtgtcggaatccctgattgtattgatcaaggaaaagccgccatctcagaggctgtatcgtatctattttcataa

SEQ ID NO: 188 - DNA, 1413 bp, Bacillus pumilus
  source 1..1413: mol_type = genomic DNA
atgcatgacaatcaaaaacaccttgtcatcattggcggtggcatcactggtttagccgccgccttctatttggaaaaggaagtcgaagaaaaaggtcttcccattcaaatatctcttattgaagcgagccctaggctaggtggaaaaatccaaacattatataaagacggctacatcattgaacgtgggcctgattcatttttagaaagaaaggtcagtggaccgcagctggcgaaagatgtaggtctatccgatcagctcgtcaataatgaaacagggcaggcgtatgtactagtcaatgaaacccttcacccgatgccaaaaggcgctgtcatgggtattccaactcaaatcagcccattcatcacaaccggtcttttttcagttgcaggaaaagcgagagccgcaatggatttcgtcttgccaaaaagcaagcaaacagaagatcagtcgctcggtgaattttttagaagacgtgtcggtgatgaagtagttgagaatttaatcgaacctcttctatcaggcatttatgcaggtgacattgaccgtctcagcttaatgtccaccttcccgcagttttatcaaacagaacaaaagcatcgcagtttgattcttgggatgaaaaaatcacagcagcatgcgaaagcgcagcaagtgacagcgaaaaaacaagggcagttccaaacgatcaatcaaggacttcaagcgcttgttgaagcagtagaaagcaagctcaagctgacaacgatttataaagggacaaaagtgaagcagattgaaaaaacagatgggggctacggtgtgcagttagacagcggtcaaacgcttttggctgattcagccattgtgacaactccgcatcaatcgatctattccatgtttccaaaagaagcggggcttgagtacttgcatgatatgacatctacttctgttgcaacggttgcactcggttttaaagaagaggatgttcataatgaatatgacggtactggttttgtcatctcaagaaacagtgatttctctattacagcttgtacgtggacgaacaaaaaatggccgcatacagctcctaaaggaaaaacattattgcgtgcttatgtagggaaggctggcgacgaatcaattgtcgaacagtcagaccatcaaatcgtcagcattgtactggaggatttgaagaaaattatggatattaaagcagatccagaactgacaacagtgactcgctggaagacgagcatgccgcaatatcacgtcggtcatcaaaaagccatttcgaacatgcgagaaacgtttaagcaatcatatcctggtgtttatatcacaggtgctgcttttgaaggtgtcggaatccctgattgtattgatcaaggaaaagctgccatttcagaggctgtatcttatctattttcataa

SEQ ID NO: 189 - DNA, 1329 bp, synthetic construct
  source 1..1329: mol_type = other DNA; note = Recombinant
atgcaaactcagcccgtcataatagcgggcgctggcattgcgggcctttctatcgcatacgagctgcaacagaagggcattccttacgaaattatggaagcctcgtcctacgccggaggcgtggtcaagtcccttcacattgatggctacgaactagacgccggacctaattcacttgccgcgtccgctgccttcatggcctacatcgaccaactcggactccaagatcaagtgcttgaagccgccgcagcatccaagaaccgcttcctcgtaagaaacgacaagctccatgcagtctcgccgcacccgtttaagatcctccagtcggcctacatcagtggcggcgctaagtggagattgtttaccgaaaggttccgcaaagctgcggctccagagggtgaggagacagtgagcagcttcgtgacgaggaggtttggcaaggagatcaacgactacctgtttgaacccgtcttgtccgggatctacgcgggcaacccggatttgatgagtgttggcgaggttctgccgatgcttcctcaatgggagcagaagtacggcagcgttacacaaggcttgttgaagaataagggcgcaatgggcggccgaaagataatcgctttcaagggcgggaatgccacactgaccaaccgtcttcagtcactgctctcaggaaagatccgcttcaattgcgccgtgacgggtgtcacacgaggcgcagacgactacattgttcagtacactgagaatggcaataccgcaatgttgaatgcaagccgcgtgatcttcacaacacccgcttactcaactgctgttgccatccaggcgttggacgccagcttggccactcacctctctgatgtaccctatcctcgcatgggtgtgttgcacttgggcttcggtgctgaggcaaggcagaaggctcctgcgggctttgggttcttggtcccacacgcagccggaaagcacttcctgggagcaatctgtaactccgctatcttcccttcgcgggtgcccactggcaaggtgttattcaccgtgttcttgggcggtgccagacaggagcaactgtttgaccagctaggccctgagaagttacaacagacagtggtgaaggagcttatggaattgctgggcctaactacgccgccggagatgcaacgattctctgagtggaatcgcgcaataccgcaacttaatgttggctacgcccagactcgtcagcagattggcgtattcgagcagcgctaccctggcatccgcttggccgggaactatgtaactggagtggcggtgcccgccattatccaagctgcaaagggctattgctaa

SEQ ID NO: 190 - DNA, 1320 bp, synthetic construct
  source 1..1320: mol_type = other DNA; note = Recombinant
cagcccgtcataatagcgggcgctggcattgcgggcctttctatcgcatacgagctgcaacagaagggcattccttacgaaattatggaagcctcgtcctacgccggaggcgtggtcaagtcccttcacattgatggctacgaactagacgccggacctaattcacttgccgcgtccgctgccttcatggcctacatcgaccaactcggactccaagatcaagtgcttgaagccgccgcagcatccaagaaccgcttcctcgtaagaaacgacaagctccatgcagtctcgccgcacccgtttaagatcctccagtcggcctacatcagtggcggcgctaagtggagattgtttaccgaaaggttccgcaaagctgcggctccagagggtgaggagacagtgagcagcttcgtgacgaggaggtttggcaaggagatcaacgactacctgtttgaacccgtcttgtccgggatctacgcgggcaacccggatttgatgagtgttggcgaggttctgccgatgcttcctcaatgggagcagaagtacggcagcgttacacaaggcttgttgaagaataagggcgcaatgggcggccgaaagataatcgctttcaagggcgggaatgccacactgaccaaccgtcttcagtcactgctctcaggaaagatccgcttcaattgcgccgtgacgggtgtcacacgaggcgcagacgactacattgttcagtacactgagaatggcaataccgcaatgttgaatgcaagccgcgtgatcttcacaacacccgcttactcaactgctgttgccatccaggcgttggacgccagcttggccactcacctctctgatgtaccctatcctcgcatgggtgtgttgcacttgggcttcggtgctgaggcaaggcagaaggctcctgcgggctttgggttcttggtcccacacgcagccggaaagcacttcctgggagcaatctgtaactccgctatcttcccttcgcgggtgcccactggcaaggtgttattcaccgtgttcttgggcggtgccagacaggagcaactgtttgaccagctaggccctgagaagttacaacagacagtggtgaaggagcttatggaattgctgggcctaactacgccgccggagatgcaacgattctctgagtggaatcgcgcaataccgcaacttaatgttggctacgcccagactcgtcagcagattggcgtattcgagcagcgctaccctggcatccgcttggccgggaactatgtaactggagtggcggtgcccgccattatccaagctgcaaagggctattgctaa

SEQ ID NO: 191 - DNA, 1347 bp, synthetic construct
  source 1..1347: mol_type = other DNA; note = Recombinant
atgagcgaccagcccgtcctcatcgttggagctggtctctccgggctctcaatcgcttacgaactacagaagctgcaagtcccttaccaagtgctggaggtttctggacattctggtggagtcatgaagtcactccggaaggacggatttgaactcgacgctggtgccaacaccatagccgcgtctcccgagattcttgcgtactttacctcactaggtcttgagaatgagatcctccaggcgactgctgcttctaaacaccgcttcttggtgcggcgaaggcaactgcacgccgtgagcccgcacccgttcaagatcatgtcatcgccgtacctcagccgtggctccaaatggcggctctttactgagcggtttcggaagcccgtcgtcgcttcgggcgaggagaccgtcaccgatttcatcacgaggagattcaaccgcgaaatagcggagtatgtgttcgaccctgttctaagcgggatctacgccgggaacccggaccaaatgagtattgctgaggtgttgcctgccttgcctaggtgggaaagggagtacggatcagtgaccaagggccttatgaaggataagggtgcgatgggaggtcgaaagatcatcagctttaagggtggcaaccagctacttacaaaccgcttacagcagctactcactactccggtgagattcaattgcaaggtgacagggattacagccagcaatggcgggtacatcgtgagcgctgttgaggacggcgtatctgagagctacaccgcatctcgtgtgatcttgaccacacccgcttactcagcagcggctaccataactaaccttgatgcagccactgcggcactgttgaacgaaatccattatccacgtatgggcgtgttacacttgggctttgatgcaactgccttgccacagccgctggacgggttcggatttctagtgccgaacgcggagaacatgcacttcctgggagccatctgcaatgcagccatcttcccggacaaggctccgcccggcaagatcctgtttacagtgttcctcggaggcgcacgccaggagtcgctcttcgatcagatgactcctgaggctcttcagcagcaagtcgttagtgaggtgatgagcttgttgcacttgtcagctccaccggtgatgcagcacttctcctcctggaacaaggccatccctcaattgaacgtcgggcacgtgaagttgcggcgcgcggtagaggcgttcgagaagaaataccctggaatccatctctcgggcaactacctccagggagttgcaataccagctttactccagcacgccgcagctttagctgcttctcttaagaagaactga

SEQ ID NO: 192 - DNA, 1413 bp, synthetic construct
  source 1..1413: mol_type = other DNA; note = Recombinant
atgtcggatggcaagaagcacgtcgtcatcataggcggtgggatcactggcttggccgctgcattctacatggagaaggagattaaggagaagaacctcccacttgagctgacgctagttgaggccagtcccagggtcggcggcaagatccagacggtcaagaaggacgggtacataattgaacgcggccctgacagcttcttagagcgcaagaaatcggctccgcagctagttaaggacttgggacttgagcacctgctcgtcaacaacgcgaccggacagtcgtacgtgctcgtgaaccggacgctccacccgatgccgaagggcgctgtgatgggcattccgaccaagatagcaccattcgtgagtaccggcctattcagcctttccggcaaggcaagggctgcgatggacttcatcttgcctgcctctaagactaaggacgatcagtccttgggcgagttcttccgccgccgggtgggtgatgaggtggtggagaacttaattgagccgctcctatctggaatctacgctggtgacatcgacaaactgtctctgatgtccacctttccgcagttctaccaaactgagcagaagcaccgttcacttatcttgggaatgaagaagactagacctcaaggttcgggtcagcaactgacggccaagaaacagggtcagttccagacgctaagcaccgggcttcagacactcgtggaggagattgagaaacagctcaaacttactaaggtgtacaagggcacgaaggtgacaaagttatcccactccggcagcgggtactccctggagttggacaatggcgtaacgttggacgccgactcagttatcgtgacagcgccgcataaggctgctgccgggatgttgtcagaactcccggcgatttcccatctcaagaacatgcacagtacctcggttgccaacgtcgccctcggattcccggaaggaagtgttcaaatggagcacgaaggcacgggtttcgtaatttccaggaactccgactttgccatcaccgcttgtacttggaccaacaagaagtggcctcatgctgcgccggagggcaagacattgctcagagcttacgtcgggaaggcgggcgacgagtcaatcgtcgatcttagcgacaacgacatcattaacattgtgctggaggacttgaagaaggttatgaacatcaatggcgagccagagatgacctgcgtgacccgatggcacgagtctatgccgcagtaccacgtcggtcacaagcagcgcatcaaggagttgcgcgaggcactcgcctcagcttaccctggcgtgtacatgactggcgcttcgtttgagggcgttggtattcctgactgcatcgaccagggaaaggcggccgtcagtgacgcgctcacctacctcttcagttga

SEQ ID NO: 193 - DNA, 1413 bp, synthetic construct
  source 1..1413: mol_type = other DNA; note = Recombinant
atgcacgacaaccagaagcacctggtcataatcggaggcggcataaccggccttgctgcggccttctacctggagaaggaggtcgaggagaagggtctccctatccagatttcattgattgaggcttcgcctcggctgggagggaagatccagacattgtacaaggacgggtacatcatcgagcgtggtccagacagtttcctggagcggaaggtcagcggaccgcagctcgccaaggacgtgggacttagcgaccaactggtgaacaacgagacaggacaggcgtacgtcttggtgaatgagaagttgcacccgatgcctaagggtgccgtgatgggcatcccaacgcaaatctcacctttcatcaccaccggactcttctccgtggccggaaaggcacgagctgcaatggacttcgttctgcctaagtcgaaacagaccgaagaccagtctctaggcgagttcttccgccgccgtgtgggtgacgaggttgtggagaacctcatcgagcctttgttgtctgggatctacgcgggcgacatcgacagacttagtctcatgagtacctttccgcaattctatcagacagaacagcagcatcgaagtctcatactcgggatgaagaagtcacaacaacatgcaaaggcccagcaagttaccgccaagaaacagggccagttccaaacgatcaaccagggcctccagagcttggtggaggcagtggagggaaagttgaagctcaccaccgtttacaaagggacaaaggttaaacagattgagaagacggacggcggttacgggttacaattggactccggacagactctcttcgctgattccgctatcgtaactactcctcaccagagcatctactctatgttcccgaaggaggcgggcctggagtacctgcacgacatgacttcaacgtctgtcgccaccgtggctttgggcttcaaggacgaggacgtccacaatgagtatgacgggacgggattcgttatcagtaggaactccgacttcagcatcaccgcctgcacgtggaccaacaagaagtggccacacaccgcgcccaaagggaagacccttctgagggcatacgtgggcaaggcgggcgacgagagcatcgtcgagcaatctgattctcagattgtttcaatcgtcctcgaagacctcaagaagatcatggacatcaaggcagacccggaacttaccaccgttactcgatggaagacctcgatgcctcagtatcacgtcgggcaccagaaggcaatcagcaacatgagggagacattcaagcagtcgtatcctggcgtgtacattaccggagcagcattcgaaggcgtaggaatccctgactgcattgaccagggcaaggctgctatctcagaggccgtgtcctatctcttctcgtga

SEQ ID NO: 194 - DNA, 1413 bp, synthetic construct
  source 1..1413: mol_type = other DNA; note = Recombinant
atgcacgacaaccagaagcacctggtgataattggaggcgggattaccggcctagcagccgctttctatctggagaaggaggtggaggagaagggcctcccgatacagatttcgctgattgaagcctctccgcgcctgggcggcaagatccagacattgtacaaggacgggtacatcattgagcgcgggcctgactcgttcctggagcggaaggtctccggtcctcaactggccaaagacgtgggtctttccgatcagcttgtgaacaatgagaccggtcaggcttacgtcttggtcaacgaaactctgcatcccatgcctaagggagccgttatgggcattccaacgcaaatctctccgttcataacgactgggctgttcagcgttgcgggcaaagcaagggctgctatggacttcgtgctgccaaagagtaagcagaccgaggaccagtccctcggcgagttcttccgccgccgagtgggcgatgaggtggttgagaatctaatcgaaccgctgttgtcgggcatctatgcgggcgacatcgacaggctaagtcttatgtccactttccctcagttctaccagacagagcagaaacacaggagtctcatccttggaatgaagaagtcccagcagcacgcgaaggctcagcaagtgaccgccaagaagcaaggacagttccagaccatcaaccagggcctacaggcccttgtcgaagccgttgagtcgaagttaaagttgacgacgatctacaagggcaccaaggtgaagcagattgagaagactgacggtggctatggtgtgcaactcgattcgggccaaacattgctcgctgactccgctatcgtcacgacgccacaccagtcgatctactcgatgttcccgaaggaggcgggcctagagtaccttcacgacatgacctccacttcggtcgccaccgttgcactcggctttaaggaggaggacgttcacaacgagtacgatggcaccggattcgtgatctccaggaactcggacttctcgattaccgcgtgcacgtggacaaataagaagtggccgcacacagcgccaaagggcaagacccttctgcgggcgtatgtgggcaaggccggtgacgagagcattgtcgaacaatctgaccatcagatcgtttctattgttcttgaggatctcaagaagataatggacattaaggccgaccctgagcttaccacagtgacgaggtggaagacctcgatgccgcagtatcacgtagggcaccagaaggccatctccaacatgcgggagacattcaagcagtcgtaccctggcgtgtacattactggcgctgctttcgagggcgttggcatcccggactgcatcgaccagggcaaggccgcaatctcagaggcagtgtcgtacctgttcagctag

SEQ ID NO: 195 - DNA, 1329 bp, synthetic construct
  source 1..1329: mol_type = other DNA; note = Recombinant
atgcaaacgcaaccagtgataatcgcgggcgctggcatcgccggactttccattgcgtacgagctccagcagaagggtatcccgtacgagatcatggaggcaagttcctatgccggcggcgtcgtcaagtcactgcacatcgacgggtacgagctggacgcgggacccaacagcctagccgcttccgctgccttcatggcgtacatcgaccagctggggctccaagaccaagtcctcgaggctgccgcggcgagtaaaaatcgctttctcgtgaggaacgacaagcttcacgctgtgtcaccgcaccctttcaaaatacttcaaagcgcctacatctcgggcggtgctaagtggcggttattcacggagcgttttaggaaggccgccgctcccgaaggtgaggagactgtttcctctttcgtcacacgcaggttcggaaaggagatcaacgattatctctttgagcctgttctcagcggaatatacgccggcaacccagaccttatgagcgtcggagaggttctccctatgctgccgcaatgggagcaaaagtatggttctgtgacccaaggcctactgaagaataagggggcgatgggcggaagaaagataattgcattcaaggggggtaatgccacccttacaaatcgcctgcaaagccttttgtcgggaaaaatccgtttcaattgtgccgtcaccggtgttacaagaggcgcagatgattacatcgttcagtacaccgagaacggtaataccgccatgctaaacgcatctagggtgattttcacaaccccggcctactcaactgccgtcgccatccaagccctcgacgccagcctggccactcatctcagtgatgtgccttaccctcgtatgggggtattacatcttggcttcggggccgaagcgcgacagaaagcccccgctggatttggcttcctagtccctcacgccgccggtaaacattttcttggcgccatctgtaactccgcaatcttcccatccagagtgcctactggcaaggttctgtttactgtgttcctgggcggtgcccgccaggagcagctattcgaccaattaggcccagaaaagctccaacaaaccgttgtgaaggaactaatggagttgctcggactgacgacaccacccgagatgcagaggttttctgagtggaaccgcgcgattccacaactcaacgtcgggtacgcccagacccggcaacagataggggttttcgagcagcgctatccaggcattcgacttgctggtaattacgtcacaggagtcgctgtgccagccataatacaagctgcaaaggggtattgctga

SEQ ID NO: 196 - DNA, 1347 bp, synthetic construct
  source 1..1347: mol_type = other DNA; note = Recombinant
atgtcagaccaaccagtcttgattgttggggccggcctctctggcctgtcgattgcctacgaactgcagaagctccaggtgccgtaccaagtcctggaggtgtcgggccatagcggcggtgtcatgaaatcgctgcgtaaggacggcttcgagttggacgcgggcgcgaacacaatcgcggctagcccagaaatacttgcttactttacaagtctgggtctggagaatgagatcctccaggctacagccgctagcaaacatcgattcctggtgcgcaggcgacaactgcacgccgtcagtccacatccattcaagataatgtcgagcccctatttaagccgcgggtccaagtggaggctctttactgaaagatttcgaaaaccggtcgtcgctagcggagaagaaactgttacagattttattactcgcaggttcaacagggagattgcagaatatgtcttcgatccagttctctcaggaatttacgcgggcaacccagaccagatgagcatcgctgaagtcctgcccgcgctccctcggtgggaacgagaatatggaagcgtcaccaaaggtctcatgaaggacaagggggccatgggcggtcggaagatcatatcgtttaaaggcgggaaccagcttctgactaaccggctgcaacagctgctcactacaccagtgcggtttaattgcaaagtcacaggtataacggctagtaatggcggctacattgtttcagcggtcgaagatggtgtgagcgagtcatacaccgcctcccgcgtgatccttaccacaccggcctactcggcggcagctacaatcaccaatcttgacgcggctacagccgcattactcaacgagattcattatcccaggatgggggtcctccatctgggcttcgacgcgacagctcttccccagcccttggatggcttcgggtttctggtcccgaacgccgaaaacatgcattttctcggcgccatttgcaacgccgcgatcttcccggataaggccccgcctggaaaaatattgttcactgtctttcttggcggcgcacgccaggagtccctgttcgaccaaatgaccccagaggctctgcagcagcaggtggtctctgaggtgatgtcacttctgcacctttctgcacctccagtgatgcagcacttctcaagctggaataaagctatcccccagttgaacgtcggccacgtgaagcttcgtagggcggtcgaagcgttcgaaaagaagtatccaggcattcacctgtccggcaactatctgcagggcgtcgcaatcccggcgctactccagcacgccgctgcgctagccgcgtctcttaagaagaattag

SEQ ID NO: 197 - DNA, 1413 bp, synthetic construct
  source 1..1413: mol_type = other DNA; note = Recombinant
atgagtgacgggaagaagcacgttgtgataatcggaggcgggataaccggcctcgccgccgccttctatatggagaaggaaattaaggagaaaaacctcccgctagagctgacgttggtggaagcgtcaccaagggtcggcggtaagatccagaccgtcaaaaaggatggctacatcatcgagcgcggcccggacagcttcctcgagcggaagaagtccgcaccccagttagtcaaagacctcggcttggaacaccttttggtcaacaacgcgacaggtcagtcctatgtgcttgtgaatcggacgctgcacccgatgcctaagggcgctgtcatgggtatccccacgaagatcgcgccgttcgtatcgaccggcctgttctccctatcaggtaaggcccgcgctgccatggactttatcctccctgcctcgaaaactaaagacgatcagtcactaggcgagttctttcggcggcgagtgggtgacgaggtggtggagaacctcatagaacccctgctgtccgggatctacgctggagacatcgacaagctgagcctcatgtctacttttccgcaattttatcagaccgagcagaaacacagatctcttatccttggcatgaagaagaccaggcctcaggggtcgggtcaacagctcacagcaaagaagcaagggcagttccaaaccctgagcacaggcttgcagaccctggtcgaagaaattgagaagcagctgaaattaacgaaggtttacaagggaaccaaggtcaccaaacttagtcacagcggctcgggctacagcctagagcttgacaacggagtgactctggacgcagacagcgtgatcgtgacggcgccccacaaggctgcggcgggaatgctgagtgagctccccgccataagtcatctcaagaacatgcactcgacgtcggtagccaatgtcgcgttggggtttcccgagggtagcgtccaaatggaacacgaaggaactggtttcgtcatatcccggaactctgacttcgcgatcacagcgtgcacttggacgaataaaaagtggccgcacgcagcgcctgaggggaagacccttcttcgagcgtatgtgggcaaagcgggcgatgaaagcattgtggatttatcggacaacgacattatcaacatcgtactggaagacctaaagaaagtcatgaacataaacggcgaaccggagatgacatgcgtcactaggtggcacgagagcatgccgcagtaccacgtggggcacaagcagcgcatcaaggaattgagggaggccctcgctagcgcgtaccctggagtttacatgaccggcgccagttttgagggtgtcggtatccctgactgtatcgaccagggtaaggccgcggtaagcgacgcattgacgtacctgttctcatga

SEQ ID NO: 198 - DNA, 1413 bp, synthetic construct
  source 1..1413: mol_type = other DNA; note = Recombinant
atgcacgacaaccagaagcatctggtcatcattggcgggggcatcacgggcttggcagccgccttctacctggagaaggaggtcgaggagaagggccttccgattcaaatatctctgattgaggcgtctccccgactcggcgggaagatccagaccctctataaagacggctatataattgagcggggaccagattctttcctggagcgaaaggtctcgggcccacagttggcgaaagatgtcggcctctccgatcaactcgtgaacaacgagaccgggcaggcctatgttctggtgaacgagaaattgcatcctatgcctaagggggccgtcatgggaataccaacccaaatatctcccttcataacaaccggactgttctcggttgccggtaaggccagggccgcgatggacttcgtcctgccaaagtctaagcagacggaggaccagtccctcggggaatttttccgccgccgggtcggcgacgaggttgtggaaaacctgattgagccgttgctgtctggcatctacgcaggcgatatcgacaggctgagccttatgtctacgttcccgcaattttatcagaccgagcagcagcaccggtctctgatacttggcatgaagaaatcacaacagcacgccaaagcacaacaggttactgctaagaagcaaggacaattccagacaatcaaccaagggttgcagtccctcgtggaggcggtagaaggcaaattgaaactcaccaccgtctacaagggcacgaaagttaagcagatcgagaaaacggatggcgggtacggtctccagctcgatagcggccagacactgttcgccgactcagcgatcgtcaccaccccccaccagtccatctacagcatgttccctaaggaggcggggttagaatacttacatgacatgacctccacctccgtcgccacagtagctctcggcttcaaggacgaggacgtgcacaacgaatacgacggtaccgggttcgtgatctcgcggaattcggacttcagtattactgcctgcacctggacgaacaagaagtggccacacacagcacccaaaggtaagaccttgctgagggcttatgtgggtaaggcgggggacgagagcatagtggagcagtctgactcgcagatcgtcagcatcgtactggaagacctgaagaagatcatggacatcaaggccgacccggagttgaccaccgtcacacggtggaaaacctcaatgccacaatatcatgtcggacatcagaaggccatctccaacatgcgcgagaccttcaagcagtcttacccgggcgtgtatatcaccggagcggctttcgagggggtcggcatccctgactgcatagaccaggggaaggcggccatcagcgaggctgtgtcgtaccttttctcgtga

SEQ ID NO: 199 - DNA, 1413 bp, synthetic construct
  source 1..1413: mol_type = other DNA; note = Recombinant
atgcatgacaaccagaagcacctggttatcattggcggtggcataaccgggctcgccgccgccttttacctggagaaggaggtggaggaaaaaggcctcccaatccagatcagtttgatagaggcgagtccgcgcttggggggcaagatccagactctgtataaagatggatacattatcgagaggggtccagacagcttcctggagcgcaaggtctccgggcctcagttggcgaaggatgttgggttgtcagatcagctcgtgaacaacgaaacgggccaggcgtatgtgttagtcaatgaaactctgcaccccatgcccaagggcgcggtgatggggatacccacccagatcagtcccttcatcacaaccggtctgttctcggtcgcagggaaggcccgagcggcgatggattttgtcctgcccaagtcgaagcagaccgaggaccagagcctcggggagtttttcaggcgcagagttggcgatgaggtcgtcgagaacctcattgagccgcttctcagcgggatttatgcgggagacatcgacaggctctccctgatgtcaacttttccgcagttctaccagacggagcaaaagcacaggagcctgattctgggaatgaagaagtcacaacaacatgctaaagcccagcaggtaactgcaaagaagcagggtcagttccaaacaatcaatcaaggtctccaggcactcgtcgaggccgtggagtcaaagctaaagctgaccaccatatacaagggtaccaaagtgaaacaaatcgagaagacagacggcggttacggagtgcagcttgactccggccagaccctcctcgccgactctgcgatcgtgaccacgccgcaccagtccatctactctatgttccccaaggaggccgggctcgaatatttgcacgatatgaccagcaccagcgtcgctacggtagcactcgggttcaaggaggaggacgtccacaacgagtacgatggcactggcttcgtgatcagccgtaactctgatttcagcatcactgcatgcacatggactaataagaaatggccccacactgcacccaagggcaagacgctgctgcgagcctacgtcgggaaggccggggacgagtctattgtagagcagagcgatcaccagattgtgagtatcgtactggaggacctgaaaaagatcatggatataaaggcggacccagagctgactaccgtgacccgctggaaaacatccatgccgcaataccatgtgggccaccaaaaagcgatctccaacatgcgggagacgttcaagcaatcttatcccggcgtgtacatcacgggagccgcgttcgagggcgtgggcatcccggattgcatcgatcagggtaaggctgcgatatcggaggctgtcagttacctgttttcttag

SEQ ID NO: 200 - DNA, 1434 bp, Paenibacillus macerans
  source 1..1434: mol_type = genomic DNA
gtgagcaaaaaaatcgccgtcatcggcggaggcataaccgggttaagcgtggcttattacgtgcgtaaattgctgcgtgaacagggggtaaacgctggggttaccctcgtggaacagtccgatcggctgggcggcaaaatccgttccctacgacgtgacggctttacgatagaacagggcccggattcaatgatcgcgcgcaagcccgccgcgctggaattgatccgggaactcgggctggaggataagctggcgggaacgaatccgcaggcgaagcgaagttatatattgcatcgcggcaaattccatcccatgccgccggggctgatgctcggcataccgacgcaaatgtggccgatggtcaagacggggctgctctctccggccggcaagctgcgggccgcgatggatctgctgcttcccgcgcggcgcggcggcggcgacgaatcgctcggcggcttcatccgccgccggctcggcagagaagtgctggagcagatgacggagccgcttctagccggcatatatgccggggacaccgaacagcttagcttgaaagcgacgtttccgcagtttatggagatggagcgcaagcaccgcagcctgatccttgggctgctggccggcaaaaagcagccgccgcggccggggggaagccaggtcccgctgccgaaggccgcgcaaaccagcatgtttctgacgttgacgggcggtttggagggactgacggaagcgctggaggaatcgctaagcgaagagaaaataattaccggccaggcggtaaccggactgtcgcagcaagaggcgggttatgagcttaacttaagcgggggcgagcgtttgaacgcggacggagtcattttggcagttcctgcttttgctgcggcccggctattggatggcgttcccgaagccgcttacctggagcggatccgttatgtgtccgtggccaatttagccttcgcctaccggcgggaagacgttccgcacgatttgaacggctccggcgtgcttatcccgcgcggggaggggcgaatgattacggccattacctgggtttcttcgaaatggctgcattcggctcccggcgataaagcgctgctgcgagcctatatcggccgcctgggcgacgaggcatggaccgcgatgtgcagggccgacatcgagcgccgggtggccgccgagctgcgcgatttgctgggcatcgccgccagcccgctgttttgcgagctcgccgctttgccggagtcgatgccccaatatccggtcgggcatgtcgagcggcttgaggcgctgcgcggggcattgtgccgggcgaagccggggctgctgctgtgcggcgcgggatatgccggcgtaggcattcccgactgcatccggcagggcaaggaagccgctgaaagcatggcggcttatttgagggatggacggtga

SEQ ID NO: 201 - DNA, 1482 bp, Paenibacillus thiaminolyticus
  source 1..1482: mol_type = genomic DNA
atgaaagctctgcggaaacttgtcgttatcggtggcggaattacgggattgagcgcggcgttctatgcgctgaagcaggcggatgaagaggggcagcccatctccgttaccatcatagagcaatcggaccgtctcggcgggaagatacagaccctgcggaaggaagggtgtgtcattgagaaaggcccggactccttcctcgcccggaagctgccgatgatcgatttggcgcgcgacctcggaatggattctgaattggtcgccacgaatccgcatgccaaaaaaacatatatattgcgccggggcaagctgtaccggatgccgcccggcctcgtgctgggcatcccgacggagctggggccgttcgcgaagacagggctcatctccccgtgggggaagctgcgcgcggctatggatctgttcatcaagccgcatccggcggatgaagatgaatccgttggcgcgttcctggacagacggctcggacgcgaagtgacggagcatattgccgagccgctgcttgccggcatttatgccggagatttgcaggcgctgagcctgcaggccaccttcccgcagttcgcgcaggtggagcggaagcacggtggcctgatacgcggaatgaaggcgagccgccaagcaggccaatcggtaccggggctgccggatgtcgccaaaggaacgatgttcctgacattccgcaacggcttgacctcgctcgtcgaacggctggaggagacgctgcgggaccgggccgaattgtgccttggcatcggcgcggaaggattcgagaagcgggaggacggaacgtatctggtgcgcttgagcgatgggagcaggctgcaggcggatgccgtcatcgtgacgacgccttcgtatcatgcggcatccttgctcgaggagcatgtcgatgcgagcgccttgcaggcgatccgtcatgtatccgtcgcgaatgtcgtcagcgtgttcgatcgcaagcaggtcaataatcagttcgacggcacagggttcgtcatctcgcgccgggaaggccgggcgattacggcctgcacgtggacctcggtgaagtggccgcatacgagccgcggggacaagcttattatccgctgctacattggccgggccggtgacgaggaacgggtggactggccggacgaggcgctcaagcggacggtgcgcagcgagctgcgggagctgcttgatatcgatatcgacccggagttcgtcgagattacgcgccttcgccactcgatgccccagtatccggtcggccatgtgcaggcgatccgctcgctgagggacgaggtggggcgcacgctcccaggcgtgttcctggcaggacagccgtacgaaggggtcggcatgcccgattgcgttcgcagcggccgcgatgcggcggaagccgcggttagcgcgatgcaggccatgagtacggagccagaggcgccagccgaggatgccgctactggaacggcggggtaa

SEQ ID NO: 202 - DNA, 1425 bp, Paenibacillus polymyxa
  source 1..1425: mol_type = genomic DNA
atgggtgataagaaacgccgtgttgttgttgtcggcggtggccttaccggcctcagcgcggcattttatatccgcaagcattaccgggaagcaggagttgaacctgtgattactttggtcgagaaaagctcgtccatgggaggcatgattgagacactgcaccgggatggatttgtgattgaaaaagggcccgattcgttcctggctcgcaaaacggcaatgattgatctggccaaagaattggagatcgatcatgagctggtaagtcagaatccggagtcgaagaaaacgtatatcatgcagcgtggcaagcttcatcctatgccagcaggacttgttctcggtattccgacagaactaagaccattcttgagaagtggtttggtttctccggcaggcaaactgcgggcgttgatggattttgtcatcccgccgcgtcgtacaacagaggatgaatcgctcggttatatgattgaacgccgtcttggagcagaagtgctggagaacttgacggaaccactgctcgcaggaatctatgcaggtgatatgcggcgattgagcctccaggctaccttcccgcagttcggagaagtagagcgcgattacggcagcttgatccggggcatgatgacggggcgcaaaccggctgagacgcataccggaacaaaacggagcgcttttttgaactttcgccagggacttcagagccttgttcatgcactcgtccatgagttgcaggatgtggatcaacgtctgaacactgcggtgaaatcgctgcaacgccttgatggagcgcagaccagataccgtgttgaacttggtaatggcgaaatgcttgaagccgatgatgtagtggttactgtgccgacatatgtcgcgtcggagctgttgaagcctcacgtggacacagcggcactggatgcgattaactatgtgtctgtagccaatgtagtgctcgcttttgagaaaaaagaggtggagcatgtattcgacggatcgggtttcctcgttccgcggaaagagggtcggaatattacggcttgcacgtggacatcgacgaaatggctgcataccagcccggatgataaagtactgcttcgctgttatgttggtcgctccggtgacgaacagaacgtagagcttccggatgaagcgctgacgaatctcgttctcaaagatctgagagagacgatgggtattgaagcagtgccgatcttctccgagattacaaggcttcgtaaatccatgccacagtatccggtgggacaccttcaacatattgccgctctccgtgaggagcttggcagcaaattaccgggtgtgtacattgcaggtgcaggttatgagggcgtaggcttgcctgattgcatcagacaagcgaaggaaatgtctgttcaggctacacaagagcttgcagcagattaa

SEQ ID NO: 203 - DNA, 1413 bp, Bacillus atrophaeus
  source 1..1413: mol_type = genomic DNA
atgagtgacggcaaaaagcatcttgtcatcatcggcggcggcatcacgggattggcctccgccttctatatggaaaaagaaatcagagagaaaaatttgcctctttctgtgacgttagtcgaagcaagcccgagagttggcgggaaaattcaaacggcccgcaaggacggttatattattgaaagagggccggactcatttttagaaagaaaaaaaagcgcaccggagcttgtcgaagatttaggccttgagcatttgcttgtcaacaatgcgacggggcagtcttatgtgctggttaacgaaacgcttcacccgatgccaaagggcgctgttatgggcatacctactaaaatagcgccatttatgtctaccggcttattttcattttccggcaaagcgcgcgcggctatggatttcgttttgcccgcaagcaagccgaaggaagatcagtccctgggtgaattcttccgcaggcgtgtcggtgacgaagttgttgaaaatttgattgagccgctattatccggcatttatgcgggtgacattgacaggctcagcctgatgtcgacgttcccgcagttttatcagaccgaacaaaagcacagaagcttgatcctcggcatgaaaaaaacaaggcctcagggctccggacagcggttaacggctaaaaaacaagggcaattccaaaccttaaagaccggcttgcagacactcgtcgaagagctggaaaaccagctgaagctgacgaaggtatacaagggtacaaaagtaaccaatatcagccgcggggaaaagggctgctccatcgctcttgataacgggatgacgctggatgccgatgcagcgattgtaacctcaccgcacaaatcggctgccggaatgtttccggatctgccagctgtcagtcagttaaaagacatgcactctacctctgtggcgaatgtcgcgcttggctttccacaagaggctgtccaaatggaacatgaaggaacgggttttgtcatctcaagaaacagtgatttttcaataacggcctgtacttggacgaataaaaaatggccgcactctgctccggaaggcaaaacgctcctcagggcttatgtcggaaaagcgggtgatgaatcaatcgtcgaactgtctgataatgagattatcaaaattgtattagaagacctaaagaaagtcatgaaaatcaaaggcgaacctgaaatgacgtgcgtcacacgctggaatgagagtatgccccaatatcatgtcggccacaaacagcgtataaaaaaagtgcgcgaagcactggctgcttcctatccgggagtttacatgacgggcgcttcattcgaaggcgttgggattccggactgtatcgatcaagggaaaagcgccgtttcagacgtacttgcttatttattcggttga

SEQ ID NO: 204 - DNA, 1413 bp, Bacillus atrophaeus
  source 1..1413: mol_type = genomic DNA
atgagtgacggcaaaaagcatcttgtcatcatcggcggcggcatcacgggattggcctccgccttctatatggaaaaagaaatcagagagaaaaatttgcctctttctgtgacgttagtcgaagcaagcccgagagttggcgggaaaattcaaacggcccgcaaggacggttatattattgaaagagggccggactcatttttagaaagaaaaaaaagcgcaccggagcttgtcgaagatttaggacttgagcatttgcttgtcaacaatgcgacggggcagtcttatgtgctggttaacgaaacgcttcacccgatgccaaagggcgctgttatgggcatacctactaaaatagcgccatttatgtctacccgcttattttcattttccggcaaagcgcgcgcggctatggatttcgttttgcccgcaagcaagccgaaggaagatcagtccctgggtgaattcttccgcaggcgtgtcggtgacgaagttgttgaaaatttgattgagccgctattatccggcatttatgcgggtgacattgacagactcagcctgatgtcgacgttcccgcagttttatcagaccgaacaaaagcacagaagcttgatcctcggcatgaaaaaaacaaggcctcagggctccggacagcagttaacggctaaaaaacaagggcaattccaaaccttaaagaccggcttgcagacactcgtcgaagagctggaaaaccagctgaaactgacgaaggtatacaagggtacaaaagtaaccaatatcagccgcggggaaaagggctgctccatcgctcttgataacgggatgacgctggatgccgatgccgcgattgtgacctcaccgcacaaatcggctgccggaatgtttccggatctgccagctgtcagccagttaaaagacatgcactctacctctgtggcgaatgtcgcgcttggctttccacaagaggctgtccaaatggaacatgaaggaacgggttttgtcatctcaagaaacagtgatttttcaataacggcctgtacttggacgaataaaaaatggccgcactctgctccggaaggcaaaacgctcctcagggcttatgtcggaaaagcgggtgatgaatcaatcgtcgaactgtctgataatgagattatcaaaattgtattagaagacctaaagaaagtcatgaaaatcaaaggcgaacctgaaatgacgtgcgtcacacgctggaatgagagtatgccccaatatcatgtcggccacaaacagcgtataaaaaaagtgcgcgaagcactggctgcttcctatccgggagtttacatgacgggcgcttcattcgaaggcgttgggattccggactgtatcgaccaagggaaaagcgccgtttcagacgtacttgcttatttattcgaatga

SEQ ID NO: 205 - DNA, 1434 bp, synthetic construct
  source 1..1434: mol_type = other DNA; note = Recombinant
atgtcaaagaagattgcagtcattggtggtgggataacagggttgtccgtggcctactacgtgaggaagctgcttcgggagcaaggcgttaatgcgggcgttaccctcgtcgagcaatccgaccgcctcggcgggaagattagatccttgagacgagacggctttaccattgagcaaggccctgactctatgattgcacgtaagcccgcagctctcgaacttatccgtgagcttggtctggaggacaagttggcgggcacaaaccctcaagccaaacgctcctacatactgcaccgtggcaagtttcatccgatgccacctgggctgatgctcgggattcccactcaaatgtggccaatggtcaagaccgggctgctatctccggccggaaagctacgggctgcgatggacctacttcttcctgcaaggcgcggaggcggcgacgaatcacttggtgggtttatccggaggcggcttggacgtgaggtgttggagcagatgaccgaaccactccttgctggaatctatgctggcgacacagaacagctttcacttaaagcgacctttcctcaattcatggagatggaaaggaaacatcgcagtctcatccttggactattggctgggaagaaacagccaccgcgtcccggtggtagccaagtgccgctcccaaaggccgctcagaccagtatgttcttgacactcaccggcgggttggaaggtctgaccgaagcactagaggaaagcctatcagaggagaagataattactggccaagcagttaccggactttcgcagcaagaggccgggtatgagttaaatctctctggcggagagagacttaatgcagacggagtgatcctcgcagtcccagcgttcgctgccgcccgacttcttgacggcgtgcctgaggccgcctacctagagcgcatccgctatgtcagtgttgctaatttggcgttcgcttacaggcgtgaggacgtgcctcatgatctgaatgggtccggcgtgttaatccctagaggtgaagggaggatgattacggccataacttgggtttcgtccaaatggttgcattcagcacccggtgacaaggcactgctgagagcgtacattgggcgactaggtgatgaggcttggacagccatgtgtagggccgacatcgagcgtagagtcgccgctgaactccgcgatctactaggaattgccgctagtcctttgttctgtgaactagccgcactcccagaatctatgccgcagtatccagtgggtcacgtcgaacgactcgaagccttgcgaggagcattgtgtcgcgctaaaccagggttgttgttgtgtggtgccgggtacgctggcgttggcattccagactgcattcggcaaggcaaagaagccgctgagtcgatggcggcttatttgagggacggacgctag

SEQ ID NO: 206 - DNA, 1482 bp, synthetic construct
  source 1..1482: mol_type = other DNA; note = Recombinant
atgaaggctctgaggaaacttgtggtcatcggcggagggatcactgggctttcggccgccttctatgcactaaagcaagccgatgaggaagggcagcccatctcggtcaccataattgaacagagcgataggctcggcggaaagatccagacactccgcaaggagggctgcgtaattgagaagggcccggattccttcctcgctaggaagttgccgatgattgatctagctcgggatcttggcatggactccgaattggtggcgactaatccgcacgcaaagaagacttacatcttgaggcgcggaaagctctaccggatgcctccaggcttagtgcttggcatacctacggaactaggaccattcgctaagacagggctcattagcccttggggcaaactccgcgccgctatggatttgttcattaagcctcatccagccgatgaagacgaaagtgttggcgctttcctggacagacgtctcggtagggaagtgaccgagcacattgcggaacctttattggcgggcatctacgcgggcgacttgcaagccttaagccttcaagccactttcccacagtttgcacaagtagagcgcaagcacggagggctgatacgcggtatgaaggccagcagacaggccggtcagtccgtgcctgggctgccggacgtcgccaagggtacgatgttccttacctttcgcaacgggcttaccagcttagttgaaaggttggaggaaactctcagagacagggctgaactctgtctgggcatcggcgcagaagggtttgagaaacgtgaagatggaacataccttgttcgactaagcgatggttcgaggctccaggccgacgcagtaattgtcactacgccgagctatcatgcggcatccctgttggaggagcatgtggatgcttcggccctccaggccattcgtcatgtaagcgttgcaaatgtcgttagcgtcttcgaccgaaagcaagtgaataaccagttcgacggcacagggtttgttatctcacggcgagaaggtcgcgcaatcaccgcctgtacctggacatccgtgaaatggccgcatacttcgcgcggcgacaaactgattatccggtgctacatcggtagggctggcgacgaggagcgagtggattggcccgatgaagctctcaagcgtactgtaagatcagaactgcgtgagttgctggacattgacattgatccggaatttgtggagattacacgactcaggcactctatgcctcaatacccagtcggccacgtccaggctatccgctctttgagggacgaggtcggtaggactttaccgggcgtgttccttgctgggcaaccctacgaaggtgtgggaatgcctgactgtgtgaggtccggccgggatgccgccgaagcagcagtaagtgctatgcaagcaatgagtacagaaccagaagcaccggcagaggacgccgctactggaacggcgggttga

SEQ ID NO: 207 - DNA, 1425 bp, synthetic construct
  source 1..1425: mol_type = other DNA; note = Recombinant
atgggagacaagaagcggagagttgttgttgttggcggcggcttgactggcctaagcgccgccttctacatccggaaacattatcgagaagctggagttgagcccgtcatcacgcttgttgagaaatctagctcgatgggagggatgattgagacccttcatagggacgggtttgtcatcgagaagggcccggacagtttcttggcacggaagaccgcaatgattgatctggcgaaagagctggagattgaccacgagttggtcagccagaatccagaatcgaagaagacctacataatgcaacgtggaaagctgcaccctatgccagcgggacttgttctgggcattcccaccgaattgcgtccctttctccggagcgggcttgtctcacccgctgggaagttgcgggcgctgatggacttcgtaataccgccacgaaggacgaccgaagatgagtcactcgggtacatgatcgagcgccgactgggtgccgaggtgttggagaacctcacagagccgttgctcgctggaatctacgctggcgacatgagaagattgtccctccaggctacgtttccgcagttcggtgaggtggagcgcgactacggctccttaatcagaggaatgatgaccggacgtaagcctgcggagacacacacagggaccaagaggtctgcctttctcaatttcagacagggtctgcaatcactggttcacgccttagtccatgaactccaggatgtagatcagaggttaaatactgcggtgaagtcgcttcagaggcttgacggcgcacaaacccgttatcgcgttgaactcggcaatggcgaaatgcttgaggctgacgacgtggtggttactgtaccaacctacgtggcgagcgagcttcttaagccgcacgtggacacggcggcgttagacgctattaactatgtgtcggtggctaatgtagttcttgcattcgagaagaaggaagtagagcacgtcttcgatggatcgggcttcttggtgcctcggaaggagggaaggaacataaccgcctgcacctggacttcgaccaagtggctccacacatcaccagatgacaaggttctgttacgttgttacgtgggcagaagtggagatgagcagaatgtggaactcccggatgaggcactcactaatctggtgcttaaggatctgagagagacgatgggcatcgaggcggttccaatcttctcagagattacccggctccgcaagtcaatgccgcagtacccagtaggacatctccagcacatcgccgcattgcgcgaggaactcggctctaagctaccaggagtgtacatcgccggagcgggctacgagggcgttggtcttccggattgcattcgccaggccaaagaaatgtcagtccaggcaacgcaagaactcgctgccgactga

SEQ ID NO: 208 - DNA, 1413 bp, synthetic construct
  source 1..1413: mol_type = other DNA; note = Recombinant
atgagtgacgggaagaagcacttggttataatcggggggggaataaccggcctggccagcgctttctatatggagaaggagatccgggagaagaacttacctctcagcgtgaccttggtggaggcatccccgcgggtaggggggaagatccagactgctcgaaaggacggctatatcatagagcggggcccggacagcttcctggagcgcaagaagtcggcgcccgagttagtcgaggacctcggtctcgagcacttactcgtaaacaacgctacagggcagtcttacgtcctcgtcaacgaaacactgcacccgatgcccaaaggcgcggtgatgggaatccccactaagattgcacctttcatgtcgactggccttttcagcttcagtgggaaggcgagggcggcaatggacttcgtcctgcccgcgtccaagccgaaggaggatcagagcctcggcgagtttttccgcaggcgagttggggatgaggtcgtggaaaacctcattgagcccttgctatccggaatctatgccggagacatcgacaggctcagccttatgtctactttcccccagttctaccagacagagcaaaagcaccgaagtttgatcctcgggatgaagaagacgcgtcctcagggttctggtcagaggctaacagcaaagaagcagggtcaattccagacgcttaaaacagggcttcaaacacttgtggaggaactcgagaatcagcttaaactaaccaaagtgtacaagggcacgaaggtaactaacatcagccgcggtgaaaagggctgcagcatcgcacttgacaacgggatgacactggacgcggacgcagcaatcgtcacgagcccccacaaatcagcggcgggaatgttccccgaccttccggcggtcagccagctgaaagacatgcactccacctccgtcgcaaacgtcgcgctcggcttcccgcaggaggctgtccagatggagcatgaggggactggcttcgttatcagcagaaattcggacttcagtatcacagcgtgcacttggacaaacaagaaatggcctcacagcgcacctgaggggaagacacttttgcgagcgtacgtggggaaagctggggacgagtccatagttgaactaagcgacaacgagataattaagatcgtgcttgaggaccttaagaaagtgatgaagataaagggcgagcccgaaatgacatgcgtaactagatggaatgagtccatgccacagtaccacgtcgggcacaagcagcgtatcaaaaaggtcagggaggctttggcggcctcatacccgggcgtatacatgaccggtgcatccttcgagggggtggggataccagactgcatcgaccaaggcaaatccgcagtctcagacgttttggcatacttgttcggctag

SEQ ID NO: 209 - DNA, 1413 bp, synthetic construct
  source 1..1413: mol_type = other DNA; note = Recombinant
atgtcggatggcaagaagcacctcgtcatcatcggcgggggtatcaccggacttgcgtccgcgttctacatggagaaggagatcagggagaagaacttgcccctctcagtgaccctggtggaggcctcgccccgtgttggtggtaagatccagacagcgcgaaaagacggctacattatcgagcgggggcccgactccttcctcgagaggaagaagtctgcccccgagcttgtggaggacttggggcttgagcacctcctcgtgaacaatgcgaccgggcagagctacgttttggtgaacgagaccctgcacccgatgcccaagggagccgtgatggggatccctaccaagatcgcgcctttcatgagcactcgacttttttcattcagcggcaaggccagagccgctatggactttgttctcccggcttctaagcctaaggaagaccagagtctaggcgaattcttcaggcgaagagtcggcgatgaggttgttgagaaccttatagagccattattgtcaggtatatacgcaggagacattgacaggctgtctctcatgagtaccttccctcaattctaccagacggagcagaaacacaggagcctcatattggggatgaagaagacgcgtcctcaaggaagcggacagcagttgacggccaagaagcagggccagttccaaacgctcaagaccggacttcagaccctcgtcgaggagcttgagaaccagctaaagttgacgaaggtttacaagggcactaaggtcacaaacatctcgaggggcgagaagggatgcagcatcgcgttagacaacgggatgaccctagacgctgacgcagctattgtgactagcccccataagtccgcagccggcatgtttccagacttgccggccgttagccagttgaaggacatgcactcgaccagcgtggcaaacgtcgcattgggcttcccacaggaggcggtgcagatggagcatgaggggaccggattcgtgatctcaaggaattccgatttctccattacggcatgtacctggacaaacaaaaaatggccccacagcgccccagaagggaaaacactcctacgcgcttatgttggcaaggccggcgatgagtcaattgtggagctctccgacaatgagatcatcaaaatcgttcttgaagatcttaagaaggtaatgaagattaagggggaaccggaaatgacgtgtgtgacaaggtggaacgagagtatgccccaatatcacgtgggccacaagcagaggataaagaaggtgagggaggcgttggcggcgtcttaccccggcgtgtacatgacgggggcttcattcgagggggtgggcatccccgactgcattgaccaaggcaaaagcgcggtgtctgacgtgctcgcgtacctgttcgagtag

SEQ ID NO: 210 - DNA, 1413 bp, synthetic construct
  source 1..1413: mol_type = other DNA; note = Recombinant
atgtccgacgggaagaagcacctggtaatcatcggtggtgggatcaccggtctggcttcagcgttctacatggaaaaggagatccgggagaagaacttgcccctttcggtgactctagtggaggcctctccacgggtggggggcaagattcagaccgcgcgcaaggatggctacatcatagagcgaggaccagactcattcctagagcgtaagaagtccgccccagagctcgtcgaggatctcggtctagagcacttgctagtgaataacgctacaggacagtcctacgtgctcgtgaacgagacactacacccgatgcctaagggggctgtcatgggtataccgaccaagatcgccccgttcatgtccactcgccttttctcgttctcgggcaaagctcgggccgctatggatttcgtcttgcctgcctcgaaaccgaaggaggaccagtccttaggagagttcttccgccggagggtcggcgacgaggtggtggagaacttaatcgaacccttgctctcggggatctacgctggagacattgatcgactatcgcttatgtctacgtttcctcaattttaccagacggagcagaagcaccgtagcctcattttgggtatgaagaagacacggcctcaaggttcggggcagcagcttactgccaagaagcagggccaattccagacactcaagaccggcttgcagactctagtggaggagctggagaatcaattgaagctgacaaaggtctacaagggtaccaaggtgacaaacatatcgcgtggcgaaaagggatgctccattgccctcgacaacggtatgaccctcgacgccgacgcagcgattgtgacgagcccacacaagagcgccgcgggcatgttcccggacttgcctgcagtgtcacagctgaaagacatgcattctacatccgtcgccaacgtcgccctgggctttccccaggaggctgtgcagatggagcacgaggggacgggcttcgttatcagccgcaactccgacttttctattaccgcgtgcacatggaccaacaagaagtggccgcacagcgctccggaggggaaaacacttctccgagcatacgtaggcaaggccggggacgagtcaattgttgagctctccgacaatgaaatcattaaaatagttctggaggatcttaagaaggtaatgaagataaagggggaacctgaaatgacgtgtgttacccgctggaatgagtcaatgccccagtaccatgtgggacacaagcagaggataaagaaggtgagggaggcgctcgctgcgtcctacccaggggtctacatgacaggagcgagttttgagggggtgggtattcccgactgtatcgaccagggtaagtcggcagtgtctgacgtgctcgcttacctattcgagtag

SEQ ID NO: 211 - DNA, 1434 bp, synthetic construct
  source 1..1434: mol_type = other DNA; note = Recombinant
atgtcgaagaagatcgccgttatcggtggaggcattacagggctctcggtcgcctactacgtgcgtaagctgcttcgtgagcaaggcgtcaacgctggtgtgacgctggttgagcagtctgatcgcctcggtggaaaaatccgtagccttcgcagagacgggttcacgattgaacaaggaccagattccatgatcgcgcgcaaacccgcggcgttggagctaattcgagaactcggactcgaggacaagctcgccggcactaacccacaggcaaagcggtcgtacatccttcaccgcgggaagttccacccgatgcccccaggcctgatgctcggcatcccgacccagatgtggccgatggtcaagaccgggctcctgtctcccgcggggaaactaagggccgctatggacctcctcctccctgctcggaggggcggcggtgatgagagtctcgggggatttatcaggcggagattaggccgcgaggtacttgagcaaatgaccgaaccactgctcgcaggtatctatgcaggcgatacggaacaactgtccttgaaagcaacatttccacaattcatggagatggaaagaaaacataggtccctcatactcggtcttcttgctggaaaaaagcaacctccgagacccggtggttcacaagtgcctctgcctaaagcggcgcaaacttcaatgttcctgactctgacaggcgggctcgaaggccttaccgaagctctagaggaatccttgtctgaggaaaaaataatcaccggccaggctgttaccgggcttagccaacaggaagccggttatgaactgaacctttcaggtggagagaggttgaacgccgatggggtcatattggctgtaccggcgttcgccgcggctcgcctgctggacggcgtccctgaggccgcgtatttggagcgcatacgctatgtttctgttgcgaacctcgcttttgcatatagacgggaagatgtgccccatgatcttaatggttccggagtgttgatcccacgcggggagggtcgaatgataacggcaattacttgggtttccagcaagtggttacattcggctcctggggataaagctcttttgcgggcatacatcggacgtctcggcgacgaagcctggacggccatgtgcagagccgacattgagcgacgggtcgctgcagagctgagagacttgttgggcatagctgcatctccattgttctgcgagctggctgcattgcctgaaagcatgccgcaatatccagtagggcatgtggagcgcctcgaagctctccgaggcgcgttgtgtagggcgaaacctggactgctgctctgcggtgccggctatgcaggtgtgggaattcctgactgtatcaggcaaggtaaagaagcggcagagtccatggccgcttaccttagggatgggcgctag

SEQ ID NO: 212 - DNA, 1482 bp, synthetic construct
  source 1..1482: mol_type = other DNA; note = Recombinant
atgaaggcgctgcggaagctggtggtaatcgggggggggatcacggggctgtcggccgcgttctacgcactcaagcaggccgacgaagagggtcagccaatttccgtaacgattatcgagcaatccgatcgacttggcggcaagatacagaccctgagaaaggagggatgcgtcattgaaaagggaccagattcatttctggcgaggaagctccccatgatcgatctggcgagagacttaggcatggactcggagctggtggccacaaatcctcatgcaaaaaagacttacatcctacggcgcggtaagttgtaccgcatgccaccgggcctggtgttggggattcctaccgagttaggacccttcgcgaaaaccggactcatcagcccctgggggaaacttcgagccgcgatggaccttttcatcaaaccacatccagccgatgaagatgagtctgtgggagcttttttagatagacgtttaggtcgcgaggtgacggagcacatcgcagagccgctgctcgccgggatatacgcaggcgatcttcaagctttgtccttgcaagctacgttccctcagttcgcgcaagtggaacgcaaacacggaggtctcatcagaggtatgaaagcgtctcgccaagctggacagtcagtcccagggctcccagatgtggccaagggtaccatgtttcttactttcagaaatggtttgactagcctggtggagcgtctcgaagaaacccttcgagatagagccgagctctgtctgggtatcggtgcagaggggtttgaaaaacgggaagacggcacgtaccttgttcgattatctgatggctccagattgcaagccgacgccgttatagttaccacaccatcataccatgccgcctccctactggaggagcacgtcgacgccagcgcgttacaggctatccgccacgtatctgtagccaacgtggtgagcgttttcgataggaagcaggttaacaatcagtttgatgggacaggttttgttatctcaagacgcgaaggcagggctatcactgcttgcacttggacctcagttaagtggccgcataccagccggggggataagttgataatccggtgttacattggtcgtgcaggagatgaggagcgcgtggattggccagacgaagcgctaaagcggaccgtgagaagtgagcttcgcgagctgttagacatagacatagatcccgaattcgtggaaattacacggttgaggcactctatgccacaataccctgttggtcatgtgcaagctatacggtccctgcgcgacgaagtaggccggaccttgccgggcgtgtttcttgcgggtcagccgtatgagggggttgggatgccagattgtgtgcgttctggccgcgacgcggcagaggctgccgtatcagccatgcaagccatgtcgacagaacccgaagccccggcggaagatgcagcgacaggaactgcaggttag

SEQ ID NO: 213 - DNA, 1425 bp, synthetic construct
  source 1..1425: mol_type = other DNA; note = Recombinant
atgggggataagaagaggagggtcgttgtcgtgggtgggggactgaccggactatcagccgcgttctacattagaaagcactaccgagaggccggcgtggagccggtgatcacgctggtcgagaagtcgagttcgatgggcggaatgatcgagaccctacacagggacggctttgtgattgagaagggaccagatagcttccttgcacgcaagacagccatgatcgatctcgcaaaagagctcgagatagaccacgaactggtgtctcagaacccggagtccaagaagacatatatcatgcagagaggtaaactacaccccatgccagccgggttggttctaggaatacctaccgagctccgcccgtttttgcgtagcggtctcgtgagccccgccgggaagctgcgtgcgctaatggacttcgtgatcccgcctcggcgaacgaccgaagacgaatcgctgggatacatgattgaacggcgattgggcgctgaggtgcttgaaaatcttacggagcctctgcttgcagggatttatgcgggtgatatgaggcggttgtctctccaggcaacgttcccacagttcggtgaggtagaacgcgattacggctcactgatacggggcatgatgaccggtcgcaagcctgccgagacacacaccggtacaaaaaggtcagcctttcttaatttccggcaagggttacagtcacttgttcatgcacttgtacacgaattgcaggacgtcgatcaaagacttaataccgcagtgaagagcctgcagcgcctggatggggcccaaactaggtaccgtgtggaattaggcaatggagagatgctggaggccgatgacgtggtggtcaccgtcccaacgtacgtagcttctgagctcctcaagccccacgttgacaccgcagctctggatgcaatcaattatgtgagcgtggctaatgtcgtcctggcctttgagaagaaggaagtggagcatgtgttcgacggatcagggttcttggttccgagaaaagagggcaggaatatcacggcgtgcacttggacttcgacaaaatggctccacacctccccggatgacaaagtacttctgcgatgctatgtgggccgaagtggtgatgagcagaatgtagagctccccgacgaggcactgaccaacctcgtcctcaaggacctaagggagactatgggcattgaggccgtgccaattttctctgaaataacacgcctgcgcaagtccatgccccaataccctgtgggccatcttcaacacattgcggccctgcgggaagaacttgggtctaagctgccgggcgtgtacatagcgggcgccggttacgagggtgtcgggttgcctgactgtattagacaggcaaaggaaatgtccgtgcaagcaacccaagaacttgctgctgactga

SEQ ID NO: 214 - DNA, 1413 bp, synthetic construct
  source 1..1413: mol_type = other DNA; note = Recombinant
atgagtgacggtaagaagcatttggtcatcatcggcggcggcatcaccggcttagcctccgccttctacatggaaaaggagattcgggagaagaaccttcccttgtcagttaccctggtggaggcctcgccacgggtcgggggtaaaatccagacggcccggaaggatggttatattatcgagcgcggacccgactcgttcctcgagcgcaagaagagcgcacccgaactcgttgaggaccttggcctcgaacatctcctcgttaacaatgcaactggtcagtcgtacgtcctggtcaacgagacactccatcccatgcccaagggcgcggtgatgggcattccgacgaagattgccccttttatgtcgactggccttttcagcttctcgggaaaggcccgtgccgctatggacttcgtcctccctgcctcgaaaccgaaggaggaccagtctcttggagaattttttaggcgcagagtgggggacgaggttgtggagaatctgatcgaaccgcttctgagcggaatctatgcgggcgacattgaccgcctctcactcatgagcaccttcccacaattctaccagacggagcagaagcatcggtcactcatcctggggatgaaaaaaacccggcctcaaggatcaggacaaaggcttacagctaaaaagcaggggcagtttcaaactctcaagacgggcctgcagactctagtcgaggagttagaaaaccagttgaagttgaccaaggtgtacaagggcacgaaagtgacaaacatcagccggggcgaaaagggttgttcaatcgcgttggacaacggcatgaccctggacgcagacgcagcaatcgtgacatcgccccacaagagtgctgcgggcatgttccctgatctgccggcggtcagccagcttaaggatatgcactcaacctcggtggctaacgtggccttgggcttccctcaggaggccgtccaaatggagcacgaaggaaccggctttgttatcagccgtaacagtgacttctcgattaccgcttgtacctggacgaacaagaagtggcctcacagcgcgccagaagggaagaccctcctgcgagcctacgtcggcaaggctggtgacgagtcgatcgttgagttgtctgacaacgagattatcaagatcgtacttgaagatctcaagaaggtcatgaagataaagggtgaacccgagatgacttgcgttactagatggaacgagtctatgcctcagtatcacgtggggcacaagcagaggatcaagaaggtccgggaggccttggctgcctcgtatccgggagtctacatgaccggggcctcatttgagggagtcggtatccccgactgcatcgaccaaggaaagtccgccgtctctgacgtgttggcttatctattcggctag

SEQ ID NO: 215 - DNA, 1413 bp, synthetic construct
  source 1..1413: mol_type = other DNA; note = Recombinant
atgagcgacggaaagaaacatctcgtgatcatcgggggcggaataacaggcctagcctcggcattctacatggagaaggagatcagagagaaaaacctcccgctctctgtgaccctggtggaggcttcaccgagagtgggcgggaagatacagacggcgcgcaaggatggctacataatagagcggggcccagattctttcctggagagaaaaaaaagcgccccggaattggtggaggacctcggcctcgaacacctcctggtgaataacgcaacagggcaaagctacgtactcgttaatgagactctccaccccatgccaaaaggggccgtgatgggaatccccacaaagatcgctccattcatgagcaccaggttattctctttctctggtaaagctagggcagccatggacttcgtcctgccagcctccaaaccgaaagaagaccaaagcctcggggaattcttccgccggagggtgggcgacgaggtggttgagaatttaattgaacctctcctctcaggtatatacgcaggggacatcgaccgcttgtcgctgatgagcacctttccgcagttctaccagacggagcagaagcatcgctcactcattcttggtatgaagaagactcgtccgcaagggtctggccagcagctgacagccaagaaacaggggcagttccaaactcttaagaccggcctacagactctggtggaggagctcgagaaccagctgaagctcacaaaggtttacaagggcacaaaggtgacaaacatctcaaggggggagaagggttgctccatcgcgctcgataacggcatgacactcgatgctgatgcggcgatagtaactagcccgcacaagtcggccgcgggaatgttccccgacctccccgcggtctcgcaactgaaggacatgcattccaccagcgtcgccaacgtagctctaggctttcctcaggaggcagtccaaatggaacacgagggcacgggtttcgtaatctcccgcaacagcgacttctcaatcactgcttgcacgtggactaacaagaagtggccgcattcggcccccgagggcaagacgcttcttcgagcatacgtgggtaaggctggtgatgagagtatcgtcgagctctcggacaacgagatcattaagatcgtgttggaggacttgaagaaggtgatgaaaatcaagggggagccggaaatgacttgcgtgactcgctggaacgagagcatgccgcagtaccacgttgggcataagcagaggataaagaaagttcgcgaagcgctggccgcgtcttaccctggagtgtatatgacgggagcctcctttgagggtgtggggatcccggactgcatcgaccagggaaagtcagctgtctccgacgtgctggcctacttattcgagtga

SEQ ID NO: 216 - DNA, 1428 bp, synthetic construct
  source 1..1428: mol_type = other DNA; note = Recombinant
aagaagattgcagtcattggtggtgggataacagggttgtccgtggcctactacgtgaggaagctgcttcgggagcaaggcgttaatgcgggcgttaccctcgtcgagcaatccgaccgcctcggcgggaagattagatccttgagacgagacggctttaccattgagcaaggccctgactctatgattgcacgtaagcccgcagctctcgaacttatccgtgagcttggtctggaggacaagttggcgggcacaaaccctcaagccaaacgctcctacatactgcaccgtggcaagtttcatccgatgccacctgggctgatgctcgggattcccactcaaatgtggccaatggtcaagaccgggctgctatctccggccggaaagctacgggctgcgatggacctacttcttcctgcaaggcgcggaggcggcgacgaatcacttggtgggtttatccggaggcggcttggacgtgaggtgttggagcagatgaccgaaccactccttgctggaatctatgctggcgacacagaacagctttcacttaaagcgacctttcctcaattcatggagatggaaaggaaacatcgcagtctcatccttggactattggctgggaagaaacagccaccgcgtcccggtggtagccaagtgccgctcccaaaggccgctcagaccagtatgttcttgacactcaccggcgggttggaaggtctgaccgaagcactagaggaaagcctatcagaggagaagataattactggccaagcagttaccggactttcgcagcaagaggccgggtatgagttaaatctctctggcggagagagacttaatgcagacggagtgatcctcgcagtcccagcgttcgctgccgcccgacttcttgacggcgtgcctgaggccgcctacctagagcgcatccgctatgtcagtgttgctaatttggcgttcgcttacaggcgtgaggacgtgcctcatgatctgaatgggtccggcgtgttaatccctagaggtgaagggaggatgattacggccataacttgggtttcgtccaaatggttgcattcagcacccggtgacaaggcactgctgagagcgtacattgggcgactaggtgatgaggcttggacagccatgtgtagggccgacatcgagcgtagagtcgccgctgaactccgcgatctactaggaattgccgctagtcctttgttctgtgaactagccgcactcccagaatctatgccgcagtatccagtgggtcacgtcgaacgactcgaagccttgcgaggagcattgtgtcgcgctaaaccagggttgttgttgtgtggtgccgggtacgctggcgttggcattccagactgcattcggcaaggcaaagaagccgctgagtcgatggcggcttatttgagggacggacgctag

SEQ ID NO: 217 - DNA, 1470 bp, synthetic construct
  source 1..1470: mol_type = other DNA; note = Recombinant
aggaaacttgtggtcatcggcggagggatcactgggctttcggccgccttctatgcactaaagcaagccgatgaggaagggcagcccatctcggtcaccataattgaacagagcgataggctcggcggaaagatccagacactccgcaaggagggctgcgtaattgagaagggcccggattccttcctcgctaggaagttgccgatgattgatctagctcgggatcttggcatggactccgaattggtggcgactaatccgcacgcaaagaagacttacatcttgaggcgcggaaagctctaccggatgcctccaggcttagtgcttggcatacctacggaactaggaccattcgctaagacagggctcattagcccttggggcaaactccgcgccgctatggatttgttcattaagcctcatccagccgatgaagacgaaagtgttggcgctttcctggacagacgtctcggtagggaagtgaccgagcacattgcggaacctttattggcgggcatctacgcgggcgacttgcaagccttaagccttcaagccactttcccacagtttgcacaagtagagcgcaagcacggagggctgatacgcggtatgaaggccagcagacaggccggtcagtccgtgcctgggctgccggacgtcgccaagggtacgatgttccttacctttcgcaacgggcttaccagcttagttgaaaggttggaggaaactctcagagacagggctgaactctgtctgggcatcggcgcagaagggtttgagaaacgtgaagatggaacataccttgttcgactaagcgatggttcgaggctccaggccgacgcagtaattgtcactacgccgagctatcatgcggcatccctgttggaggagcatgtggatgcttcggccctccaggccattcgtcatgtaagcgttgcaaatgtcgttagcgtcttcgaccgaaagcaagtgaataaccagttcgacggcacagggtttgttatctcacggcgagaaggtcgcgcaatcaccgcctgtacctggacatccgtgaaatggccgcatacttcgcgcggcgacaaactgattatccggtgctacatcggtagggctggcgacgaggagcgagtggattggcccgatgaagctctcaagcgtactgtaagatcagaactgcgtgagttgctggacattgacattgatccggaatttgtggagattacacgactcaggcactctatgcctcaatacccagtcggccacgtccaggctatccgctctttgagggacgaggtcggtaggactttaccgggcgtgttccttgctgggcaaccctacgaaggtgtgggaatgcctgactgtgtgaggtccggccgggatgccgccgaagcagcagtaagtgctatgcaagcaatgagtacagaaccagaagcaccggcagaggacgccgctactggaacggcgggttga

SEQ ID NO: 218 - DNA, 1410 bp, synthetic construct
  source 1..1410: mol_type = other DNA; note = Recombinant
cggagagttgttgttgttggcggcggcttgactggcctaagcgccgccttctacatccggaaacattatcgagaagctggagttgagcccgtcatcacgcttgttgagaaatctagctcgatgggagggatgattgagacccttcatagggacgggtttgtcatcgagaagggcccggacagtttcttggcacggaagaccgcaatgattgatctggcgaaagagctggagattgaccacgagttggtcagccagaatccagaatcgaagaagacctacataatgcaacgtggaaagctgcaccctatgccagcgggacttgttctgggcattcccaccgaattgcgtccctttctccggagcgggcttgtctcacccgctgggaagttgcgggcgctgatggacttcgtaataccgccacgaaggacgaccgaagatgagtcactcgggtacatgatcgagcgccgactgggtgccgaggtgttggagaacctcacagagccgttgctcgctggaatctacgctggcgacatgagaagattgtccctccaggctacgtttccgcagttcggtgaggtggagcgcgactacggctccttaatcagaggaatgatgaccggacgtaagcctgcggagacacacacagggaccaagaggtctgcctttctcaatttcagacagggtctgcaatcactggttcacgccttagtccatgaactccaggatgtagatcagaggttaaatactgcggtgaagtcgcttcagaggcttgacggcgcacaaacccgttatcgcgttgaactcggcaatggcgaaatgcttgaggctgacgacgtggtggttactgtaccaacctacgtggcgagcgagcttcttaagccgcacgtggacacggcggcgttagacgctattaactatgtgtcggtggctaatgtagttcttgcattcgagaagaaggaagtagagcacgtcttcgatggatcgggcttcttggtgcctcggaaggagggaaggaacataaccgcctgcacctggacttcgaccaagtggctccacacatcaccagatgacaaggttctgttacgttgttacgtgggcagaagtggagatgagcagaatgtggaactcccggatgaggcactcactaatctggtgcttaaggatctgagagagacgatgggcatcgaggcggttccaatcttctcagagattacccggctccgcaagtcaatgccgcagtacccagtaggacatctccagcacatcgccgcattgcgcgaggaactcggctctaagctaccaggagtgtacatcgccggagcgggctacgagggcgttggtcttccggattgcattcgccaggccaaagaaatgtcagtccaggcaacgcaagaactcgctgccgactga

SEQ ID NO: 219 - DNA, 1398 bp, synthetic construct
  source 1..1398: mol_type = other DNA; note = Recombinant
aagcacctggtaatcatcggtggtgggatcaccggtctggcttcagcgttctacatggaaaaggagatccgggagaagaacttgcccctttcggtgactctagtggaggcctctccacgggtggggggcaagattcagaccgcgcgcaaggatggctacatcatagagcgaggaccagactcattcctagagcgtaagaagtccgccccagagctcgtcgaggatctcggtctagagcacttgctagtgaataacgctacaggacagtcctacgtgctcgtgaacgagacactacacccgatgcctaagggggctgtcatgggtataccgaccaagatcgccccgttcatgtccactcgccttttctcgttctcgggcaaagctcgggccgctatggatttcgtcttgcctgcctcgaaaccgaaggaggaccagtccttaggagagttcttccgccggagggtcggcgacgaggtggtggagaacttaatcgaacccttgctctcggggatctacgctggagacattgatcgactatcgcttatgtctacgtttcctcaattttaccagacggagcagaagcaccgtagcctcattttgggtatgaagaagacacggcctcaaggttcggggcagcagcttactgccaagaagcagggccaattccagacactcaagaccggcttgcagactctagtggaggagctggagaatcaattgaagctgacaaaggtctacaagggtaccaaggtgacaaacatatcgcgtggcgaaaagggatgctccattgccctcgacaacggtatgaccctcgacgccgacgcagcgattgtgacgagcccacacaagagcgccgcgggcatgttcccggacttgcctgcagtgtcacagctgaaagacatgcattctacatccgtcgccaacgtcgccctgggctttccccaggaggctgtgcagatggagcacgaggggacgggcttcgttatcagccgcaactccgacttttctattaccgcgtgcacatggaccaacaagaagtggccgcacagcgctccggaggggaaaacacttctccgagcatacgtaggcaaggccggggacgagtcaattgttgagctctccgacaatgaaatcattaaaatagttctggaggatcttaagaaggtaatgaagataaagggggaacctgaaatgacgtgtgttacccgctggaatgagtcaatgccccagtaccatgtgggacacaagcagaggataaagaaggtgagggaggcgctcgctgcgtcctacccaggggtctacatgacaggagcgagttttgagggggtgggtattcccgactgtatcgaccagggtaagtcggcagtgtctgacgtgctcgcttacctattcgagtag

SEQ ID NO: 220 - DNA, 1422 bp, synthetic construct
  source 1..1422: mol_type = other DNA; note = Recombinant
attgcagtcattggtggtgggataacagggttgtccgtggcctactacgtgaggaagctgcttcgggagcaaggcgttaatgcgggcgttaccctcgtcgagcaatccgaccgcctcggcgggaagattagatccttgagacgagacggctttaccattgagcaaggccctgactctatgattgcacgtaagcccgcagctctcgaacttatccgtgagcttggtctggaggacaagttggcgggcacaaaccctcaagccaaacgctcctacatactgcaccgtggcaagtttcatccgatgccacctgggctgatgctcgggattcccactcaaatgtggccaatggtcaagaccgggctgctatctccggccggaaagctacgggctgcgatggacctacttcttcctgcaaggcgcggaggcggcgacgaatcacttggtgggtttatccggaggcggcttggacgtgaggtgttggagcagatgaccgaaccactccttgctggaatctatgctggcgacacagaacagctttcacttaaagcgacctttcctcaattcatggagatggaaaggaaacatcgcagtctcatccttggactattggctgggaagaaacagccaccgcgtcccggtggtagccaagtgccgctcccaaaggccgctcagaccagtatgttcttgacactcaccggcgggttggaaggtctgaccgaagcactagaggaaagcctatcagaggagaagataattactggccaagcagttaccggactttcgcagcaagaggccgggtatgagttaaatctctctggcggagagagacttaatgcagacggagtgatcctcgcagtcccagcgttcgctgccgcccgacttcttgacggcgtgcctgaggccgcctacctagagcgcatccgctatgtcagtgttgctaatttggcgttcgcttacaggcgtgaggacgtgcctcatgatctgaatgggtccggcgtgttaatccctagaggtgaagggaggatgattacggccataacttgggtttcgtccaaatggttgcattcagcacccggtgacaaggcactgctgagagcgtacattgggcgactaggtgatgaggcttggacagccatgtgtagggccgacatcgagcgtagagtcgccgctgaactccgcgatctactaggaattgccgctagtcctttgttctgtgaactagccgcactcccagaatctatgccgcagtatccagtgggtcacgtcgaacgactcgaagccttgcgaggagcattgtgtcgcgctaaaccagggttgttgttgtgtggtgccgggtacgctggcgttggcattccagactgcattcggcaaggcaaagaagccgctgagtcgatggcggcttatttgagggacggacgctag

SEQ ID NO: 221 - DNA, 1464 bp, synthetic construct
  source 1..1464: mol_type = other DNA; note = Recombinant
cttgtggtcatcggcggagggatcactgggctttcggccgccttctatgcactaaagcaagccgatgaggaagggcagcccatctcggtcaccataattgaacagagcgataggctcggcggaaagatccagacactccgcaaggagggctgcgtaattgagaagggcccggattccttcctcgctaggaagttgccgatgattgatctagctcgggatcttggcatggactccgaattggtggcgactaatccgcacgcaaagaagacttacatcttgaggcgcggaaagctctaccggatgcctccaggcttagtgcttggcatacctacggaactaggaccattcgctaagacagggctcattagcccttggggcaaactccgcgccgctatggatttgttcattaagcctcatccagccgatgaagacgaaagtgttggcgctttcctggacagacgtctcggtagggaagtgaccgagcacattgcggaacctttattggcgggcatctacgcgggcgacttgcaagccttaagccttcaagccactttcccacagtttgcacaagtagagcgcaagcacggagggctgatacgcggtatgaaggccagcagacaggccggtcagtccgtgcctgggctgccggacgtcgccaagggtacgatgttccttacctttcgcaacgggcttaccagcttagttgaaaggttggaggaaactctcagagacagggctgaactctgtctgggcatcggcgcagaagggtttgagaaacgtgaagatggaacataccttgttcgactaagcgatggttcgaggctccaggccgacgcagtaattgtcactacgccgagctatcatgcggcatccctgttggaggagcatgtggatgcttcggccctccaggccattcgtcatgtaagcgttgcaaatgtcgttagcgtcttcgaccgaaagcaagtgaataaccagttcgacggcacagggtttgttatctcacggcgagaaggtcgcgcaatcaccgcctgtacctggacatccgtgaaatggccgcatacttcgcgcggcgacaaactgattatccggtgctacatcggtagggctggcgacgaggagcgagtggattggcccgatgaagctctcaagcgtactgtaagatcagaactgcgtgagttgctggacattgacattgatccggaatttgtggagattacacgactcaggcactctatgcctcaatacccagtcggccacgtccaggctatccgctctttgagggacgaggtcggtaggactttaccgggcgtgttccttgctgggcaaccctacgaaggtgtgggaatgcctgactgtgtgaggtccggccgggatgccgccgaagcagcagtaagtgctatgcaagcaatgagtacagaaccagaagcaccggcagaggacgccgctactggaacggcgggttga

SEQ ID NO: 222 - DNA, 1404 bp, synthetic construct
  source 1..1404: mol_type = other DNA; note = Recombinant
gttgttgttgttggcggcggcttgactggcctaagcgccgccttctacatccggaaacattatcgagaagctggagttgagcccgtcatcacgcttgttgagaaatctagctcgatgggagggatgattgagacccttcatagggacgggtttgtcatcgagaagggcccggacagtttcttggcacggaagaccgcaatgattgatctggcgaaagagctggagattgaccacgagttggtcagccagaatccagaatcgaagaagacctacataatgcaacgtggaaagctgcaccctatgccagcgggacttgttctgggcattcccaccgaattgcgtccctttctccggagcgggcttgtctcacccgctgggaagttgcgggcgctgatggacttcgtaataccgccacgaaggacgaccgaagatgagtcactcgggtacatgatcgagcgccgactgggtgccgaggtgttggagaacctcacagagccgttgctcgctggaatctacgctggcgacatgagaagattgtccctccaggctacgtttccgcagttcggtgaggtggagcgcgactacggctccttaatcagaggaatgatgaccggacgtaagcctgcggagacacacacagggaccaagaggtctgcctttctcaatttcagacagggtctgcaatcactggttcacgccttagtccatgaactccaggatgtagatcagaggttaaatactgcggtgaagtcgcttcagaggcttgacggcgcacaaacccgttatcgcgttgaactcggcaatggcgaaatgcttgaggctgacgacgtggtggttactgtaccaacctacgtggcgagcgagcttcttaagccgcacgtggacacggcggcgttagacgctattaactatgtgtcggtggctaatgtagttcttgcattcgagaagaaggaagtagagcacgtcttcgatggatcgggcttcttggtgcctcggaaggagggaaggaacataaccgcctgcacctggacttcgaccaagtggctccacacatcaccagatgacaaggttctgttacgttgttacgtgggcagaagtggagatgagcagaatgtggaactcccggatgaggcactcactaatctggtgcttaaggatctgagagagacgatgggcatcgaggcggttccaatcttctcagagattacccggctccgcaagtcaatgccgcagtacccagtaggacatctccagcacatcgccgcattgcgcgaggaactcggctctaagctaccaggagtgtacatcgccggagcgggctacgagggcgttggtcttccggattgcattcgccaggccaaagaaatgtcagtccaggcaacgcaagaactcgctgccgactga

SEQ ID NO: 223 - DNA, 1398 bp, synthetic construct; identical to SEQ ID NO: 219
  source 1..1398: mol_type = other DNA; note = Recombinant

SEQ ID NO: 224 - protein, 445 aa, synthetic construct
  source 1..445: note = Recombinant
QPVLIVGAGLSGLSIAYELQKLQVPYQVLEVSGHSGGVMKSLRKDGFELDAGANTIAASPEILAYFTSLGLENEILQATAASKHRFLVRRRQLHAVSPHPFKIMSSPYLSRGSKWRLFTERFRKPVVASGEETVTDFITRRFNREIAEYVFDPVLSGIYAGNPDQMSIAEVLPALPRWEREYGSVTKGLMKDKGAMGGRKIISFKGGNQLLTNRLQQLLTTPVRFNCKVTGITASNGGYIVSAVEDGVSESYTASRVILTTPAYSAAATITNLDAATAALLNEIHYPRMGVLHLGFDATALPQPLDGFGFLVPNAENMHFLGAICNAAIFPDKAPPGKILFTVFLGGARQESLFDQMTPEALQQQVVSEVMSLLHLSAPPVMQHFSSWNKAIPQLNVGHVKLRRAVEAFEKKYPGIHLSGNYLQGVAIPALLQHAAALAASLKKN

SEQ ID NO: 225 - protein, 448 aa, synthetic construct
  source 1..448: note = Recombinant
MSDQPVLIVGAGLSGLSIAYELQKLQVPYQVLEVSGHSGGVMKSLRKDGFELDAGANTIATSPEILAYFTSLGLENEILQATATSKHRFLVRRRQLHAVSPHPFKIMSSPYLCRGSKWRLFTERFRKPVVASGEETVTDFITRRFNREIAEYVFDPVLSGIYAGNPDQMSIAEVLPALPRWEREYGSVTKGLMKDKGAMGGRKIISFKGGNQLLTNRLQQLLTTPVRFNCKVTGITASNGGYIVSAVEDGVSESYTASRVILTTPAYSAAATITNLDAATAALLNEIHYPRMGVLHLGFDATALPQPLDGFGFLVPNAENMHFLGAICNAAIFPDKAPPGKILFTVFLGGARQESLFDQMTPEALQQQVVSEVMSLLHLSAPPVMQHFSSWNKAIPQLNVGHVKLRRAVEAFEKKYPGIHLSGNYLQGVAIPALLQHAAALAASLKKN

SEQ ID NO: 226 - protein, 466 aa, synthetic construct
  source 1..466: note = Recombinant
KKHVVIIGGGITGLAAAFYMEKEIKEKNLPLELTLVEASPRVGGKIQTVKKDGYIIERGPDSFLERKKSAPQLVKDLGLEHLLVNNATGQSYVLVNRTLHPMPKGAVMGIPTKIAPFVSTGLFSLSGKARAAMDFILPASKTKDDQSLGEFFRRRVGDEVVENLIEPLLSGIYAGDIDKLSLMSTFPQFYQTEQKHRSLILGMKKTRPQGSGQQLTAKKQGQFQTLSTGLQTLVEEIEKQLKLTKVYKGTKVTKLSHSGSGYSLELDNGVTLDADSVIVTAPHKAAAGMLSELPAISHLKNMHSTSVANVALGFPEGSVQMEHEGTGFVISRNSDFAITACTWTNKKWPHAAPEGKTLLRAYVGKAGDESIVDLSDNDIINIVLEDLKKVMNINGEPEMTCVTRWHESMPQYHVGHKQRIKELREALASAYPGVYMTGASFEGVGIPDCIDQGKAAVSDALTYLFS

SEQ ID NO: 227 - protein, 465 aa, synthetic construct
  source 1..465: note = Recombinant
KHLVIIGGGITGLAAAFYLEKEVEEKGLPIQISLIEASPRLGGKIQTLYKDGYIIERGPDSFLERKVSGPQLAKDVGLSDQLVNNETGQAYVLVNEKLHPMPKGAVMGIPTQISPFITTGLFSVAGKARAAMDFVLPKSKQTEDQSLGEFFRRRVGDEVVENLIEPLLSGIYAGDIDRLSLMSTFPQFYQTEQQHRSLILGMKKSQQHAKAQQVTAKKQGQFQTINQGLQSLVEAVEGKLKLTTVYKGTKVKQIEKTDGGYGLQLDSGQTLFADSAIVTTPHQSIYSMFPKEAGLEYLHDMTSTSVATVALGFKDEDVHNEYDGTGFVISRNSDFSITACTWTNKKWPHTAPKGKTLLRAYVGKAGDESIVEQSDSQIVSIVLEDLKKIMDIKADPELTTVTRWKTSMPQYHVGHQKAISNMRETFKQSYPGVYITGAAFEGVGIPDCIDQGKAAISEAVSYLFS

SEQ ID NO: 228 - protein, 465 aa, synthetic construct
  source 1..465: note = Recombinant
KHLVIIGGGITGLAAAFYLEKEVEEKGLPIQISLIEASPRLGGKIQTLYKDGYIIERGPDSFLERKVSGPQLAKDVGLSDQLVNNETGQAYVLVNETLHPMPKGAVMGIPTQISPFITTGLFSVAGKARAAMDFVLPKSKQTEDQSLGEFFRRRVGDEVVENLIEPLLSGIYAGDIDRLSLMSTFPQFYQTEQKHRSLILGMKKSQQHAKAQQVTAKKQGQFQTINQGLQALVEAVESKLKLTTIYKGTKVKQIEKTDGGYGVQLDSGQTLLADSAIVTTPHQSIYSMFPKEAGLEYLHDMTSTSVATVALGFKEEDVHNEYDGTGFVISRNSDFSITACTWTNKKWPHTAPKGKTLLRAYVGKAGDESIVEQSDHQIVSIVLEDLKKIMDIKADPELTTVTRWKTSMPQYHVGHQKAISNMRETFKQSYPGVYITGAAFEGVGIPDCIDQGKAAISEAVSYLFS

SEQ ID NO: 229 - DNA, 537 bp, synthetic construct
  source 1..537: mol_type = other DNA; note = Recombinant
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcaactgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 230 - DNA, 1338 bp, synthetic construct
  source 1..1338: mol_type = other DNA; note = Recombinant
cagcccgtcctcatcgttggagctggtctctccgggctctcaatcgcttacgaactacagaagctgcaagtcccttaccaagtgctggaggtttctggacattctggtggagtcatgaagtcactccggaaggacggatttgaactcgacgctggtgccaacaccatagccgcgtctcccgagattcttgcgtactttacctcactaggtcttgagaatgagatcctccaggcgactgctgcttctaaacaccgcttcttggtgcggcgaaggcaactgcacgccgtgagcccgcacccgttcaagatcatgtcatcgccgtacctcagccgtggctccaaatggcggctctttactgagcggtttcggaagcccgtcgtcgcttcgggcgaggagaccgtcaccgatttcatcacgaggagattcaaccgcgaaatagcggagtatgtgttcgaccctgttctaagcgggatctacgccgggaacccggaccaaatgagtattgctgaggtgttgcctgccttgcctaggtgggaaagggagtacggatcagtgaccaagggccttatgaaggataagggtgcgatgggaggtcgaaagatcatcagctttaagggtggcaaccagctacttacaaaccgcttacagcagctactcactactccggtgagattcaattgcaaggtgacagggattacagccagcaatggcgggtacatcgtgagcgctgttgaggacggcgtatctgagagctacaccgcatctcgtgtgatcttgaccacacccgcttactcagcagcggctaccataactaaccttgatgcagccactgcggcactgttgaacgaaatccattatccacgtatgggcgtgttacacttgggctttgatgcaactgccttgccacagccgctggacgggttcggatttctagtgccgaacgcggagaacatgcacttcctgggagccatctgcaatgcagccatcttcccggacaaggctccgcccggcaagatcctgtttacagtgttcctcggaggcgcacgccaggagtcgctcttcgatcagatgactcctgaggctcttcagcagcaagtcgttagtgaggtgatgagcttgttgcacttgtcagctccaccggtgatgcagcacttctcctcctggaacaaggccatccctcaattgaacgtcgggcacgtgaagttgcggcgcgcggtagaggcgttcgagaagaaataccctggaatccatctctcgggcaactacctccagggagttgcaataccagctttactccagcacgccgcagctttagctgcttctcttaagaagaactga

SEQ ID NO: 231 - DNA, 1344 bp, synthetic construct
  source 1..1344: mol_type = other DNA; note = Recombinant
atgagcgaccaacccgtcctcatcgttggagctggtctctccgggctctcaatcgcttacgaactacagaagctgcaagtcccttaccaagtgctggaggtttctggacattctggtggagtcatgaagtcactccggaaggacggatttgaactcgacgctggtgccaacaccatagccacgtctcccgagattcttgcgtactttacctcactaggtcttgagaatgagatcctccaggcgactgctacttctaaacaccgcttcttggtgcggcgaaggcaactgcacgccgtgagcccgcacccgttcaagatcatgtcatcgccgtacctctgccgtggctccaaatggaggctctttactgagcggtttcggaaacccgtcgtcgcttcgggcgaggagaccgtcaccgatttcatcacgaggagattcaaccgcgaaatagcggagtatgtgttcgaccctgttctaagtgggatctacgccgggaacccggaccaaatgagtattgctgaggtgttgcctgccttgcctaggtgggaaagggagtacggatcagtgaccaagggccttatgaaggataagggtgcgatgggaggtcgaaagatcatcagctttaagggtggcaaccagctacttacaaaccgcttacagcagctactcactactccggtgagattcaattgcaaggtgacagggattacagccagcaatggcgggtacatcgtgagcgctgttgaggacggcgtatctgagagctacaccgcatctcgtgtgatcttgaccacacccgcttactcagcagcggctaccataactaaccttgatgcagccactgcggcactgttgaacgaaatccattatccacgtatgggcgtgttacacttgggctttgatgcaactgccttgccacagccgctggacgggttcggatttctagtgccgaacgcggagaacatgcacttcctgggagccatctgcaatgcagccatcttcccggacaaggctccgcccggcaagatcctgtttacagtgttcctcggaggcgcacgccaggagtcgctcttcgatcagatgactcctgaggctcttcagcagcaagtcgttagtgaggtgatgagcttgttgcacttgtcagctccaccggtgatgcagcacttctcctcctggaacaaggccatccctcaattgaacgtcgggcacgtgaagttgcggcgcgcggtagaggcgttcgagaagaaataccctggaatccatctctcgggcaactacctccagggagttgcaataccagctttactccagcacgccgcagctttagctgcttctcttaagaagaac

SEQ ID NO: 232 - DNA, 1338 bp, synthetic construct
  source 1..1338: mol_type = other DNA; note = Recombinant
cagcccgtcctcatcgttggagctggtctctccgggctctcaatcgcttacgaactacagaagctgcaagtcccttaccaagtgctggaggtttctggacattctggtggagtcatgaagtcactccggaaggacggatttgaactcgacgctggtgccaacaccatagccacgtctcccgagattcttgcgtactttacctcactaggtcttgagaatgagatcctccaggcgactgctacttctaaacaccgcttcttggtgcggcgaaggcaactgcacgccgtgagcccgcacccgttcaagatcatgtcatcgccgtacctctgccgtggctccaaatggaggctctttactgagcggtttcggaaacccgtcgtcgcttcgggcgaggagaccgtcaccgatttcatcacgaggagattcaaccgcgaaatagcggagtatgtgttcgaccctgttctaagtgggatctacgccgggaacccggaccaaatgagtattgctgaggtgttgcctgccttgcctaggtgggaaagggagtacggatcagtgaccaagggccttatgaaggataagggtgcgatgggaggtcgaaagatcatcagctttaagggtggcaaccagctacttacaaaccgcttacagcagctactcactactccggtgagattcaattgcaaggtgacagggattacagccagcaatggcgggtacatcgtgagcgctgttgaggacggcgtatctgagagctacaccgcatctcgtgtgatcttgaccacacccgcttactcagcagcggctaccataactaaccttgatgcagccactgcggcactgttgaacgaaatccattatccacgtatgggcgtgttacacttgggctttgatgcaactgccttgccacagccgctggacgggttcggatttctagtgccgaacgcggagaacatgcacttcctgggagccatctgcaatgcagccatcttcccggacaaggctccgcccggcaagatcctgtttacagtgttcctcggaggcgcacgccaggagtcgctcttcgatcagatgactcctgaggctcttcagcagcaagtcgttagtgaggtgatgagcttgttgcacttgtcagctccaccggtgatgcagcacttctcctcctggaacaaggccatccctcaattgaacgtcgggcacgtgaagttgcggcgcgcggtagaggcgttcgagaagaaataccctggaatccatctctcgggcaactacctccagggagttgcaataccagctttactccagcacgccgcagctttagctgcttctcttaagaagaactga

SEQ ID NO: 233 - DNA, 1401 bp, synthetic construct
  source 1..1401: mol_type = other DNA; note = Recombinant
aagaagcacgtcgtcatcataggcggtgggatcactggcttggccgctgcattctacatggagaaggagattaaggagaagaacctcccacttgagctgacgctagttgaggccagtcccagggtcggcggcaagatccagacggtcaagaaggacgggtacataattgaacgcggccctgacagcttcttagagcgcaagaaatcggctccgcagctagttaaggacttgggacttgagcacctgctcgtcaacaacgcgaccggacagtcgtacgtgctcgtgaaccggacgctccacccgatgccgaagggcgctgtgatgggcattccgaccaagatagcaccattcgtgagtaccggcctattcagcctttccggcaaggcaagggctgcgatggacttcatcttgcctgcctctaagactaaggacgatcagtccttgggcgagttcttccgccgccgggtgggtgatgaggtggtggagaacttaattgagccgctcctatctggaatctacgctggtgacatcgacaaactgtctctgatgtccacctttccgcagttctaccaaactgagcagaagcaccgttcacttatcttgggaatgaagaagactagacctcaaggttcgggtcagcaactgacggccaagaaacagggtcagttccagacgctaagcaccgggcttcagacactcgtggaggagattgagaaacagctcaaacttactaaggtgtacaagggcacgaaggtgacaaagttatcccactccggcagcgggtactccctggagttggacaatggcgtaacgttggacgccgactcagttatcgtgacagcgccgcataaggctgctgccgggatgttgtcagaactcccggcgatttcccatctcaagaacatgcacagtacctcggttgccaacgtcgccctcggattcccggaaggaagtgttcaaatggagcacgaaggcacgggtttcgtaatttccaggaactccgactttgccatcaccgcttgtacttggaccaacaagaagtggcctcatgctgcgccggagggcaagacattgctcagagcttacgtcgggaaggcgggcgacgagtcaatcgtcgatcttagcgacaacgacatcattaacattgtgctggaggacttgaagaaggttatgaacatcaatggcgagccagagatgacctgcgtgacccgatggcacgagtctatgccgcagtaccacgtcggtcacaagcagcgcatcaaggagttgcgcgaggcactcgcctcagcttaccctggcgtgtacatgactggcgcttcgtttgagggcgttggtattcctgactgcatcgaccagggaaaggcggccgtcagtgacgcgctcacctacctcttcagttga

SEQ ID NO: 234 - DNA, 1398 bp, synthetic construct
  source 1..1398: mol_type = other DNA; note = Recombinant
aagcacctggtcataatcggaggcggcataaccggccttgctgcggccttctacctggagaaggaggtcgaggagaagggtctccctatccagatttcattgattgaggcttcgcctcggctgggagggaagatccagacattgtacaaggacgggtacatcatcgagcgtggtccagacagtttcctggagcggaaggtcagcggaccgcagctcgccaaggacgtgggacttagcgaccaactggtgaacaacgagacaggacaggcgtacgtcttggtgaatgagaagttgcacccgatgcctaagggtgccgtgatgggcatcccaacgcaaatctcacctttcatcaccaccggactcttctccgtggccggaaaggcacgagctgcaatggacttcgttctgcctaagtcgaaacagaccgaagaccagtctctaggcgagttcttccgccgccgtgtgggtgacgaggttgtggagaacctcatcgagcctttgttgtctgggatctacgcgggcgacatcgacagacttagtctcatgagtacctttccgcaattctatcagacagaacagcagcatcgaagtctcatactcgggatgaagaagtcacaacaacatgcaaaggcccagcaagttaccgccaagaaacagggccagttccaaacgatcaaccagggcctccagagcttggtggaggcagtggagggaaagttgaagctcaccaccgtttacaaagggacaaaggttaaacagattgagaagacggacggcggttacgggttacaattggactccggacagactctcttcgctgattccgctatcgtaactactcctcaccagagcatctactctatgttcccgaaggaggcgggcctggagtacctgcacgacatgacttcaacgtctgtcgccaccgtggctttgggcttcaaggacgaggacgtccacaatgagtatgacgggacgggattcgttatcagtaggaactccgacttcagcatcaccgcctgcacgtggaccaacaagaagtggccacacaccgcgcccaaagggaagacccttctgagggcatacgtgggcaaggcgggcgacgagagcatcgtcgagcaatctgattctcagattgtttcaatcgtcctcgaagacctcaagaagatcatggacatcaaggcagacccggaacttaccaccgttactcgatggaagacctcgatgcctcagtatcacgtcgggcaccagaaggcaatcagcaacatgagggagacattcaagcagtcgtatcctggcgtgtacattaccggagcagcattcgaaggcgtaggaatccctgactgcattgaccagggcaaggctgctatctcagaggccgtgtcctatctcttctcgtga

SEQ ID NO: 235 - DNA, 1398 bp, synthetic construct
  source 1..1398: mol_type = other DNA; note = Recombinant
aagcacctggtgataattggaggcgggattaccggcctagcagccgctttctatctggagaaggaggtggaggagaagggcctcccgatacagatttcgctgattgaagcctctccgcgcctgggcggcaagatccagacattgtacaaggacgggtacatcattgagcgcgggcctgactcgttcctggagcggaaggtctccggtcctcaactggccaaagacgtgggtctttccgatcagcttgtgaacaatgagaccggtcaggcttacgtcttggtcaacgaaactctgcatcccatgcctaagggagccgttatgggcattccaacgcaaatctctccgttcataacgactgggctgttcagcgttgcgggcaaagcaagggctgctatggacttcgtgctgccaaagagtaagcagaccgaggaccagtccctcggcgagttcttccgccgccgagtgggcgatgaggtggttgagaatctaatcgaaccgctgttgtcgggcatctatgcgggcgacatcgacaggctaagtcttatgtccactttccctcagttctaccagacagagcagaaacacaggagtctcatccttggaatgaagaagtcccagcagcacgcgaaggctcagcaagtgaccgccaagaagcaaggacagttccagaccatcaaccagggcctacaggcccttgtcgaagccgttgagtcgaagttaaagttgacgacgatctacaagggcaccaaggtgaagcagattgagaagactgacggtggctatggtgtgcaactcgattcgggccaaacattgctcgctgactccgctatcgtcacgacgccacaccagtcgatctactcgatgttcccgaaggaggcgggcctagagtaccttcacgacatgacctccacttcggtcgccaccgttgcactcggctttaaggaggaggacgttcacaacgagtacgatggcaccggattcgtgatctccaggaactcggacttctcgattaccgcgtgcacgtggacaaataagaagtggccgcacacagcgccaaagggcaagacccttctgcgggcgtatgtgggcaaggccggtgacgagagcattgtcgaacaatctgaccatcagatcgtttctattgttcttgaggatctcaagaagataatggacattaaggccgaccctgagcttaccacagtgacgaggtggaagacctcgatgccgcagtatcacgtagggcaccagaaggccatctccaacatgcgggagacattcaagcagtcgtaccctggcgtgtacattactggcgctgctttcgagggcgttggcatcccggactgcatcgaccagggcaaggccgcaatctcagaggcagtgtcgtacctgttcagctag

SEQ ID NO: 236 - protein, 68 aa, Glycine max; identical to SEQ ID NO: 1

SEQ ID NO: 237 - protein, 56 aa, synthetic construct
  source 1..56: note = Recombinant
MATTTAAAAVTISIPKKPVFIRRPRLRGPVDCRGLHASDAIISNEAPTGTTISADC

SEQ ID NO: 238 - protein, 76 aa, Eragrostis tef
MAAAPPLAADMVLPSPCPAAVAPTPVVAAAWGAARAGSVRCKATQLRMMRTGGPVAPVAGRRRRAPLSVRCDASSR

SEQ ID NO: 239 - protein, 80 aa, Setaria italica
MAAAPPLSADALSFLPSAAAPAAAAPTPVVAAAWGAARAAGSVRGKAALRMARRGSGLAPVVGRRPRRPPLSVRCDATSR

SEQ ID NO: 240 - protein, 84 aa, Acalypha ostryifolia
MATTTATTSFSGVSICPPHQTNRTSLFPPQSLSFPSSKHGSLVNSVQFNRSRRARRNHFSLTSITNAPRRKRLLSVRCDASATS

SEQ ID NO: 241 - protein, 78 aa, Adansonia digitata
MAASSSSVVSFSGISLCSTHSISNKTYLFSAHPRISVSFPSKPNSLKSFKQLQLKKNGLFEKFSRTSSRSFVVRCDAS

SEQ ID NO: 242 - protein, 82 aa, Taraxacum officinale
MATTASFSGVRIHAPSSTCIDRTTLFAQPSVSFSSFSKPRRTTLRSLKLRSRSNDVLLRTRTGDRFGGKSSRSFVVRCDASS

SEQ ID NO: 243 - protein, 77 aa, Amaranthus cruentus
MATATTSFPGAYLRVPPKNGVRNALFSQSIVSIARNSRKPKSLKSLKLSTNSFNFGLHKSCRKGSKSGSFVVRCDAA

SEQ ID NO: 244 - protein, 80 aa, Amaranthus cruentus
MAIATTSFPGTYLRVPPKNGVRNALFSRSVVSNGVNSRKPNSLESLKSSRNSSNVCLSTSFGHYRKSSKSGSFFVRCNAA

SEQ ID NO: 245 - protein, 80 aa, Amaranthus hypochondriacus; identical to SEQ ID NO: 244

SEQ ID NO: 246 - protein, 77 aa, Amaranthus palmeri
MATATTSFPGAYLRVPPKNGVRNALFSQSIVSIALNSRKPKSFKSLKSSANSCNFGLHKSYRKGSKSGSFVVRCDAA

SEQ ID NO: 247 - protein, 80 aa, Amaranthus palmeri
MATATTSFPGTYLRVPPKNGVRNALFSRSVVSNGVNSRKPNSLKSLKLSRNSSNVCLYTSFGHYRKSSKSGSFIIRCNAA

SEQ ID NO: 248 - protein, 77 aa, Amaranthus palmeri; identical to SEQ ID NO: 246

SEQ ID NO: 249 - protein, 77 aa, Amaranthus palmeri; identical to SEQ ID NO: 246

SEQ ID NO: 250 - protein, 80 aa, Amaranthus palmeri
MATATTSFPGTYLRVPPKNGVRNALFSRSVVSNGVNSRKPKSLKSLKSSRNSSNVCLYTSFGHYRKSSKSGSFIIRCNAA

SEQ ID NO: 251 - protein, 80 aa, Amaranthus tuberculatus
MATATTSFPGTYLRVPPKNGVRNALFSRSVVSNGVDSRKPNSLKSMKLSRNSSNVCLYTSFGHYRKSSKSGSFIVRCNAA

SEQ ID NO: 252 - protein, 80 aa, Amaranthus tuberculatus; identical to SEQ ID NO: 251

SEQ ID NO: 253 - protein, 80 aa, Amaranthus tuberculatus; identical to SEQ ID NO: 251

SEQ ID NO: 254 - protein, 80 aa, Amaranthus viridis
MAIATTSFPGTYLRVPPKNGVRNALFSRSVVSNGVNSRKPNSLESLKSSRNSSNVCLSTSFGHYRKGSKSGSFVVRCDAA

SEQ ID NO: 255 - protein, 77 aa, Amaranthus viridis; identical to SEQ ID NO: 243

SEQ ID NO: 256 - protein, 71 aa, Ambrosia trifida
MSTMSTLFHLPSSLCTDRTITSSFAQPSVSVNSFSKPRRVALRSLKLKTRSNDVLLRKSSRSLVVRCDASS

SEQ ID NO: 257 - protein, 87 aa, Conyza canadensis
MATAAFSGVPCIDRTSLLSAQPSSSSSSSVVVCYSSFSKPGTTLLPSLKLKSSRNNNNSNVFLFGNTRKTSRLSFLVRCDSSSSSSS

SEQ ID NO: 258 - protein, 71 aa, Cucumis melo
MAFSTAPFYAIGIRFPSHSSSISSTTNALILKSPLALALTAKPKSPLLLKRNVGCQRFGRNSRFVVRCDAS

SEQ ID NO: 259 - protein, 78 aa, Kochia scoparia
MATATTSFPGAYLHLPPKNGVRNALFSQPICSSNLNLKKPNSLKSVKLSRSSGNALFYKNAKKNSKFGSLVVRCDAAG

SEQ ID NO: 260 - protein, 78 aa, Kochia scoparia; identical to SEQ ID NO: 259

SEQ ID NO: 261 - protein, 78 aa, Kochia scoparia; identical to SEQ ID NO: 259

SEQ ID NO: 262 - protein, 84 aa, unidentified
  source 1..84: note = Rosa hybrid cultivar; note = Rosa hybrida
MASSTTSFAASGVGLRLPQSVSTKCCSKASLFPHPTLSLTFHARPQFFRGLASRQFNPNGAFGTGSGRLGRTPNPFVVRSEASS

SEQ ID NO: 263 - protein, 80 aa, Sedum album
MAASAATITSSISAITPKPSSFSSSPSVTVPRFSVSCSAIPRPHKNPCSLKFRVKDSRFNGIVKKRSNSNSFVVRCDTSS

SEQ ID NO: 264 - protein, 79 aa, Sedum album
MAADAATITAGITLTTARRSSSSIAPQFSVCCSAITNTQKNLSFLKLRVKDATLTTRIEGIQKKRYNSASFVVRCDASS

SEQ ID NO: 265 - protein, 81 aa, Spinacia oleracea
MATATTSFLGAYLRVPPNNGVRNALFSQPFLSLRIKSKRTKSLNSLKFTGDSSKILLFKCSRPFEKGLKSGSFVVRCDAAG

SEQ ID NO: 266 - protein, 79 aa, Allium cepa
MAATSSATTHLPFFSPHTKHAKTNSFFASLPVSAYSTKNSISFKALKAVRWSETFGQSKKANGFAKRKQFAVVRCDASS

SEQ ID NO: 267 - DNA, 204 bp, Glycine max
  source 1..204: mol_type = genomic DNA
atggctactgctactaccacagctaccgctgcattctctggtgttgtgagtgttggaaccgagacacgtagaatttactctttctcacacttgcaacctagcgcagccttccctgccaagccatcatcctttaagtccttgaagctgaaacagtcggcgaggcttacgaggcgcctcgatcatagaccctttgtggtccgatgc

SEQ ID NO: 268 - DNA, 168 bp, synthetic construct
  source 1..168: mol_type = other DNA; note = Recombinant
atggccactaccacagcagccgcggcggtcaccatcagcattcctaaaaagcctgtttttatccgccgcccacgacttcgtgggcccgtcgactgcagaggcctgcatgcatccgacgcaatcatctccaacgaggcccctacagggacgacaatctcggctgactgt

SEQ ID NO: 269 - DNA, 228 bp, Eragrostis tef
  source 1..228: mol_type = genomic DNA
atggcagccgcacctcccctagcagccgacatggtgttaccatccccatgccctgccgcggttgcacctaccccagtggttgcagctgcttggggtgcagcccgagctggatctgttagatgtaaagcgacccaacttcgaatgatgagaactgggggccctgttgctccagttgccggtagacgacgacgagctccattgagtgtacgttgtgatgcttcctccaga

SEQ ID NO: 270 - DNA, 240 bp, Setaria italica
  source 1..240: mol_type = genomic DNA
atggctgccgctcctcccctctctgcagatgcactatcattcctaccatccgccgccgctccggcagccgctgcaccaacacctgttgtagctgcggcatggggagccgcacgagctgcagggtcagttagaggtaaagctgctttgcgtatggctcgaaggggtagtggactggctccagtggttggaagaagacctcgacgacctcctctttcagttagatgtgacgcaacatctcgt

SEQ ID NO: 271 - DNA, 252 bp, Acalypha ostryifolia
  source 1..252: mol_type = genomic DNA
atggctacaaccaccgcgacgacgtctttctcgggcgtctcgatctgcccacctcaccagacgaatcgcacctctttgtttccgccccagtccttgtctttcccctccagtaagcatggcagtcttgtgaactctgtgcaattcaaccgttcgcgacgcgctagacgtaatcacttcagcctcacttccattaccaatgcaccgaggcgcaaaaggttactatctgtccggtgcgacgcgagtgccacatct

SEQ ID NO: 272 - DNA, 234 bp, Adansonia digitata
  source 1..234: mol_type = genomic DNA
atggcggcgtcatcttcgtccgtcgtgagcttctcgggcatctcgttgtgcagtactcactcgatctccaacaagacctatctattctccgcccacccgcgcatttcggtgtcgttccccagtaagcccaatagtttgaagtccttcaagcagctccagctgaagaagaacggactctttgagaagttctctcgtacctccagtcggagcttcgtggtgaggtgcgacgcgtcg

SEQ ID NO: 273 - DNA, 246 bp, Taraxacum officinale
  source 1..246: mol_type = genomic DNA
atggctacaaccgcgagcttctcgggtgttcgtattcacgcgccttcctccacatgtatcgaccggaccactttattcgcccagccttcggtgagcttttcttccttttccaagccgaggcgaacgaccttgaggtcgctgaagctaaggtcgaggtccaacgatgtgttgcttcgcacccgcacaggtgacagattcggcggaaagagctcacgttcatttgttgtgcgctgcgacgcatcttct

SEQ ID NO: 274 - DNA, 231 bp, Amaranthus cruentus
  source 1..231: mol_type = genomic DNA
atggcgaccgcgacgacctcgtttcccggcgcgtacctgcgcgtgccgcccaagaacggggttcgtaacgccctctttagccagtctatcgtgtcaatagcgcgcaactctcggaaacccaaatcgctcaaatcccttaaactatctaccaactcctttaacttcggtctgcacaagtcttgtcgaaagggaagcaaatccgggtcgttcgtagtgcgttgtgacgcggcc

SEQ ID NO: 275 - DNA, 240 bp, Amaranthus cruentus
  source 1..240: mol_type = genomic DNA
atggccatcgccaccacgagctttccgggaacgtacctccgggtgccgcccaagaacggcgtccgaaacgccctattcagtcgctccgtcgtgtctaatggggtgaactcaaggaagccgaactcgctggagtcgcttaaatcgtcgaggaatagctcgaacgtctgcttgagtacgtcgttcgggcattaccggaaatcgagtaagtcgggctcgttcttcgttcggtgtaacgccgcc

SEQ ID NO: 276 - DNA, 240 bp, Amaranthus hypochondriacus
  source 1..240: mol_type = genomic DNA
atggccatcgccacgacctcgttccccggcacgtacctgcgagtgccgcccaagaacggggtccggaacgcgctgttctctcggtccgtggtcagcaatggtgttaattcacgaaagccgaacagtctggaatctctcaagagcagtcgaaactcctccaacgtctgcctttcgaccagcttcggtcactaccggaagtctagtaagagcgggtcgttctttgtccggtgtaatgctgcc

SEQ ID NO: 277 - DNA, 231 bp, Amaranthus palmeri
  source 1..231: mol_type = genomic DNA
atggcgacggccaccacctcgttccccggcgcatacctccgcgtgccgcccaagaacggcgtccgcaacgcactctttagccagagcatcgtcagtatcgcccttaacagtcgcaagcctaaatcgttcaagtcactaaagtcaagcgctaattcgtgcaactttggacttcacaagtcctaccggaaaggcagcaagtctggcagcttcgtcgttcgttgtgatgccgcc

SEQ ID NO: 278 - DNA, 240 bp, Amaranthus palmeri
  source 1..240: mol_type = genomic DNA
atggccaccgccaccacctcgttccccggcacgtacctgcgcgtgccgcccaagaacggcgtcaggaacgcgctgtttagtcgctcagtcgtgtccaacggggtgaactcacgaaagcccaacagtttaaagagcttaaaactgtcgaggaactctagtaatgtctgcctctacacctccttcggacactatcgaaagtccagtaagtcgggctcattcatcatccggtgcaatgcggcc

SEQ ID NO: 279 - DNA, 231 bp, Amaranthus palmeri
  source 1..231: mol_type = genomic DNA
atggccaccgcaacaaccagcttccctggcgcgtaccttcgtgtgccgcccaagaacggcgtccgcaatgcgctgtttagtcagtccatcgtgagtatcgctctcaattcccggaaacctaagagctttaagagcctaaagtcgagcgctaattcttgcaacttcgggcttcacaagagctatcggaaagggtctaagagcgggtcattcgtcgtgcggtgcgacgcggcc

SEQ ID NO: 280 - DNA, 231 bp, Amaranthus palmeri
  source 1..231: mol_type = genomic DNA
atggccacggccacgacctcgtttccgggtgcgtacctgcgagttccgcccaagaacggtgtacggaacgccttgttctcccaatccatcgtgagcatcgccctcaacagtcgcaaaccgaagtcattcaaatccctgaaaagttcggccaatagctgtaacttcgggctgcataagagttaccgcaaggggtcgaaatccgggtcgttcgtcgtccggtgcgacgctgcg

SEQ ID NO: 281 - DNA, 240 bp, Amaranthus palmeri
  source 1..240: mol_type = genomic DNA
atggcaactgccacgacgtcctttccgggaacgtatctccgcgtgccgcccaagaacggcgtccgcaacgccctgttctcacgatccgtcgttagcaatggcgtcaatagccgcaagcctaagtccctgaaatcgctcaagtcgtcgcgcaactctagtaatgtctgtctctacacatcgttcggacattaccgcaaatcatccaaatccggctcgttcataatccggtgcaatgcggct

SEQ ID NO: 282 - DNA, 240 bp, Amaranthus tuberculatus
  source 1..240: mol_type = genomic DNA
atggcgacagcgaccacatccttccctggcacttacctgagagtgccgcccaagaatggggtgagaaacgccttgttcagccgcagcgtagtctctaatggggtggatagtcgcaaaccgaatagcctcaagagtatgaagctcagccgcaacagctcaaatgtctgcctctacacgagctttggccactaccgaaagtcctccaagtctgggtcgttcatcgtgcgctgtaacgccgcg

SEQ ID NO: 283 - DNA, 240 bp, Amaranthus tuberculatus
  source 1..240: mol_type = genomic DNA
atggccacggccaccacctcctttcctggcacatacctccgcgtccctcccaagaatggggtgcgaaacgcactctttagtagatcggtcgtttccaatggtgtcgattcccgcaagccgaactccctcaagtcgatgaagctgtcccgcaactcatcgaacgtttgcctctatacctcgtttgggcactaccgcaagtcgagcaaatcgggctcgttcattgtccggtgtaatgcagcc

SEQ ID NO: 284 - DNA, 240 bp, Amaranthus tuberculatus
  source 1..240: mol_type = genomic DNA
atggcgacggcgacaacctcgtttccgggaacgtacctgcgcgtgccgcccaagaacggggtgcggaacgccctgttcagccgctccgtcgtgtccaatggcgtcgattcgaggaagcctaactcattgaaatctatgaagttgtctcgtaattccagcaacgtttgcctctacacctcgttcgggcattaccgcaagtcaagcaagtccggatcgtttatcgtgcggtgcaacgctgcg

SEQ ID NO: 285 - DNA, 240 bp, Amaranthus viridis
  source 1..240: mol_type = genomic DNA
atggccattgccaccacgtcgtttcccggcacgtacctgagggttccgcccaagaacggagtccgcaacgcactgtttagtcgctccgtggtgagtaacggggtcaactccagaaaacctaattcgctggagtcccttaaatcgagccggaacagctcgaacgtctgcttgtcaacctcctttggccactaccggaagggctccaagtcgggctcattcgtcgtgcggtgcgatgcggcg

SEQ ID NO: 286 - DNA, 231 bp, Amaranthus viridis
  source 1..231: mol_type = genomic DNA
atggccaccgccacgacgtcctttcccggtgcgtatctgcgagtgcctcccaagaacggcgtccggaacgcgctgttcagccagtccatcgtgagcatcgcgcggaatagtcggaaacctaagtcgctcaaatccttgaaactgtcaacgaactctttcaatttcgggttgcataagtcctgccgaaagggtagcaaatccgggtctttcgttgtgcggtgcgacgcggcc

SEQ ID NO: 287 - DNA, 213 bp, Ambrosia trifida
  source 1..213: mol_type = genomic DNA
atgagtacgatgtcaaccctatttcacctcccgtctagcctgtgtaccgacaggacgatcaccagcagcttcgcacaaccgagcgtttcggtcaactcgttctcgaagccgcgccgcgtcgcgctccggtccttaaagctcaaaacgcgaagtaatgacgtcctgctgcggaaatcttcacgttcgctagtcgtgcgttgcgacgccagcagc

SEQ ID NO: 288 - DNA, 261 bp, Conyza canadensis
  source 1..261: mol_type = genomic DNA
atggcgacggccgccttctcgggcgttccgtgcattgaccggacatcactcctctccgcccagccatcgtcctcctcttccagtagcgtcgtggtctgctactcctcctttagcaagccgggcacgaccctattgccgtcgttgaagctcaaaagcagccgcaacaacaacaattcaaacgtattcctcttcgggaacaccaggaaaacatcccgtctgtcattcctagtgcgctgcgattcctcatcttcaagctctagc

SEQ ID NO: 289 - DNA, 213 bp, Cucumis melo
  source 1..213: mol_type = genomic DNA
atggcgtttagcaccgcacccttctacgcaattggtatcagatttcccagccatagctcatcaatctcaagcaccactaacgccctcatccttaaaagtccactggcgttagccctaaccgctaagccgaagtctcccctactcctcaagcgcaacgttggctgccagcgattcgggcgaaactcccgcttcgtcgtgcgctgcgatgcgtcc

SEQ ID NO: 290 - DNA, 234 bp, Kochia scoparia
  source 1..234: mol_type = genomic DNA
atggcgaccgccacgacctccttccccggcgcgtacctccatctcccgcccaagaacggggtccgcaacgccttgttctctcaacccatctgttcatccaacctcaacctcaagaaacctaactctctcaaatcggtgaagctgtcccgcagttccggcaatgccctattctacaagaacgccaagaagaatagtaagttcggcagtctggtcgtgcggtgcgacgcggcggga

SEQ ID NO: 291 - DNA, 234 bp, Kochia scoparia
  source 1..234: mol_type = genomic DNA
atggccaccgcgacgaccagctttcccggcgcgtatctgcacctcccgcccaagaacggcgtgagaaacgcgctgttcagtcaaccgatatgctcgtccaatctcaacctcaagaaacccaattctctgaaaagcgtcaaactgtcgcgtagtagcggcaatgcgctgttctacaagaacgccaagaagaatagcaagttcgggtcgctcgtggtgcgctgcgacgcggcgggc

SEQ ID NO: 292 - DNA, 234 bp, Kochia scoparia
  source 1..234: mol_type = genomic DNA
atggccacagccaccacgtccttccctggggcctacctacatctcccgcccaagaatggcgtgcgaaacgcgctgttcagtcagcctatatgcagcagtaatcttaacctcaagaagcctaattccctcaagtcagtgaaactgagccggtctagcgggaacgcgctgttctacaagaacgccaaaaagaatagcaagttcggctcgctcgtggtccggtgcgacgcggcgggc

SEQ ID NO: 293 - DNA, 252 bp, unidentified
  source 1..252: mol_type = genomic DNA; note = Rosa hybrid cultivar; note = Rosa hybrida
atggcgtcatcgaccacttcgttcgccgccagtggagttggattgcggctccctcagtccgtgagcacgaagtgctgctctaaagcgtcattgttcccacaccccacactatcgttgaccttccacgctaggccacagttctttagaggcttggcgtctcgccagttcaatccaaacggagcgtttgggacgggctccggacggctgggccggacaccaaatccgtttgtcgtcagaagcgaagcgagttct

SEQ ID NO: 294 - DNA, 240 bp, Sedum album
  source 1..240: mol_type = genomic DNA
atggcggcgagcgcggctacgatcacctccagcatatcggcgattaccccgaagccgtcgtccttctcaagcagcccttcggtcaccgtgccccgattctctgtgtcgtgcagcgcgataccgcgtccacacaagaatccctgctcgttgaagttccgggtgaaggactcacggtttaacggaattgtcaagaagcgcagtaacagcaactcattcgtagtacgttgtgacacttcctcg

SEQ ID NO: 295 - DNA, 237 bp, Sedum album
  source 1..237: mol_type = genomic DNA
atggccgccgacgcagctaccattacggcgggtatcactctcacgacggcccgccgctcctcctccagtattgcgccgcagttctcggtgtgttgctcagcgattaccaacacgcaaaaaaatctgagcttcctcaagttgcgcgtgaaagacgccaccttgactacacggattgagggtattcagaagaagcggtacaactccgcgtccttcgtcgtcagatgcgacgcgagcagc

SEQ ID NO: 296 - DNA, 243 bp, Spinacia oleracea
  source 1..243: mol_type = genomic DNA
atggccactgccacgacgtcctttctcggcgcttacttgcgggtgccgcccaacaatggcgtgaggaatgcgctgttcagtcaaccgttcctgtcgcttcgcattaagtccaaacgcactaagagcctcaactcgttgaaattcacaggagactcaagtaagattctgctgtttaagtgctcccggccgtttgagaaggggcttaaatccggctcgttcgtggtgcgctgcgacgcggccggt

SEQ ID NO: 297 - DNA, 237 bp, Allium cepa
  source 1..237: mol_type = genomic DNA
atggcggcaacgagctccgcgaccactcacctcccttttttcagcccgcacaccaaacacgcaaagacaaactctttcttcgcgtcccttccggtcagcgcctactccacgaaaaactctatcagtttcaaggcgctcaaggccgtgcgatggagcgagaccttcgggcaatcgaagaaggccaatggttttgccaaaaggaagcaatttgccgtcgtgcggtgcgatgcgagttca

(19) United States
(12) Patent Application Publication
(10) Pub. No.: US 2026/0308609 A1
(43) Pub. Date: Oct. 8, 2026

(54) METHODS AND COMPOSITIONS FOR GENE EXPRESSION IN PLANTS

(71) Applicant: Monsanto Technology LLC, St. Louis, MO (US)

(72) Inventors: Clayton T. Larue, Chesterfield, MO (US); Joel E. Ream, Edmonds, WA (US); Aabid Shariff, Durham, NC (US); Yuanji Zhang, Weldon Spring, MO (US); Xuefeng Zhou, St. Louis, MO (US)

(21) Appl. No.: 19/717,172

(22) Filed: Jun. 23, 2026

Related U.S. Application Data
(62) Division of application No. 18/182,009, filed on Mar. 10, 2023, now Pat. No. 12,692,510.
Application No. 18/182,009 is a division of application No. 16/931,427, filed on Jul. 16, 2020, now Pat. No. 11,629,358.
Application No. 16/931,427 is a division of application No. 15/660,660, filed on Jul. 26, 2017, now Pat. No. 10,745,712.
(60) Provisional application No. 62/368,840, filed on Jul. 29, 2016.

(51) Int. Cl. C12N 15/82; C12N 9/02

(52) U.S. Cl. CPC C12N 15/8274; C12N 9/001; C12N 15/8221; C12Y 103/03004

(57) ABSTRACT

The invention provides recombinant DNA molecules useful for providing efficient expression of proteins in transgenic plants, as well as compositions and methods for using the recombinant DNA molecules. In particular embodiments, the invention provides recombinant DNA molecules and constructs comprising sequences encoding transit peptides and operably linked sequences conferring herbicide tolerance.

### REFERENCE TO RELATED APPLICATION

This application is a divisional of U.S. patent application Ser. No. 18/182,009, filed Mar. 10, 2023, which is a divisional of U.S. patent application Ser. No. 16/931,427, filed Jul. 16, 2020, now U.S. Pat. No. 11,629,358, which is a divisional of U.S. patent application Ser. No. 15/660,660, filed Jul. 26, 2017, now U.S. Pat. No. 10,745,712, which claims the benefit of U.S. Provisional Serial Application No. 62/368,840, filed Jul. 29, 2016, each of which is herein incorporated by reference in its entirety.

### INCORPORATION OF SEQUENCE LISTING

A computer readable form of a sequence listing is filed with this application by electronic submission and is incorporated into this application by reference in its entirety. The sequence listing is contained in the file named MONS397USD3_ST26.xml, which is 406,167 bytes in size (measured in operating system MS Windows) and was created on Jun. 11, 2026.

### BACKGROUND OF THE INVENTION

### Field of the Invention

The invention relates generally to the fields of agriculture, plant biotechnology, and molecular biology. More specifically, the invention relates to compositions for recombinant protein expression in transgenic plants and methods of use thereof.

### Description of Related Art

Agricultural crop production often utilizes crops with modified genomes, including transgenic traits created using the methods of molecular biology. For example, a heterologous gene, also known as a transgene, can be introduced into a plant genome. Expression of the transgene in the plant confers a trait, such as herbicide-tolerance or insect control, on the plant. Successful expression of a transgene in a plant may be achieved by utilizing heterologous gene expression elements. One example of this is the use of a transit peptide operably linked to a recombinant protein to achieve sub-cellular localization of the recombinant protein and thus enhanced protein expression or function. A need therefore exists for novel transit peptides capable of effectively localizing recombinant proteins within plant cells.

### SUMMARY OF INVENTION

In one aspect, the present invention provides a recombinant DNA molecule comprising a DNA sequence encoding a transit peptide operably linked to a DNA sequence encoding a heterologous herbicide-tolerance protein, wherein the transit peptide comprises an amino acid sequence comprising at least 97 percent identity to a sequence selected from the group consisting of SEQ ID NOs:4-49 and SEQ ID NOs:236-266. In one embodiment, the heterologous herbicide-tolerance protein has herbicide-insensitive protoporphyrinogen oxidase activity. In another embodiment, the heterologous herbicide-tolerance protein comprises an amino acid sequence comprising at least 97 percent identity to a sequence selected from the group consisting of SEQ ID NOs:100-119, SEQ ID NOs:163-182, and SEQ ID NOs:224-228. In a further embodiment, the DNA sequence encoding a transit peptide comprises a nucleic acid sequence comprising at least 97 percent identity to a sequence selected from the group consisting of SEQ ID NOs:54-99 and SEQ ID NOs:267-297. In still a further embodiment, the DNA sequence encoding a heterologous herbicide-tolerance protein comprises a nucleic acid sequence comprising at least 97 percent identity to a sequence selected from the group consisting of SEQ ID NOs:121-162 and SEQ ID NOs:183-223, SEQ ID NOs:229-235. In yet a further embodiment, the recombinant DNA molecule further comprises a heterologous promoter operably linked to the DNA sequence encoding a transit peptide.

In another aspect, the present invention provides a DNA construct comprising a DNA molecule provided herein, such as a recombinant DNA molecule comprising a DNA sequence encoding a transit peptide operably linked to a DNA sequence encoding a heterologous herbicide-tolerance protein, wherein the transit peptide comprises an amino acid sequence comprising at least 97 percent identity to a sequence selected from the group consisting of SEQ ID NOs:4-49 and SEQ ID NOs:236-266, operably linked to a heterologous promoter. In one embodiment, the heterologous herbicide-tolerance protein has herbicide-insensitive protoporphyrinogen oxidase activity. In another embodiment, the heterologous herbicide-tolerance protein comprises an amino acid sequence selected from the group consisting of SEQ ID NOs:100-119, SEQ ID NOs:163-182, and SEQ ID NOs:224-228. In still another embodiment, the DNA construct is present in the genome of a transgenic plant, seed, or cell.

In a further aspect, the present invention provides a transgenic plant, seed, or cell comprising a recombinant DNA molecule provided herein, such as a recombinant DNA molecule comprising a DNA sequence encoding a transit peptide operably linked to a DNA sequence encoding a heterologous herbicide-tolerance protein, wherein the transit peptide comprises an amino acid sequence comprising at least 97 percent identity to a sequence selected from the group consisting of SEQ ID NOs:4-49 and SEQ ID NOs:236-266. In one embodiment, the plant, seed, or cell is tolerant to at least one PPO herbicide. In another embodiment, the PPO herbicide is selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, and S-3100. In a further embodiment, the transgenic plant, seed, or cell is tolerant to at least a second herbicide.

In another aspect, the present invention provides a recombinant protein comprising in operable linkage: a) a transit peptide comprising an amino acid sequence comprising at least 95 percent identity to a sequence selected from the group consisting of SEQ ID NOs:4-49 and SEQ ID NOs:236-266; and b) a heterologous herbicide-tolerance protein. In one embodiment, the heterologous herbicide-tolerance protein has herbicide-insensitive protoporphyrinogen oxidase activity. In a further aspect, the present invention provides a transgenic plant, seed, or cell comprising the recombinant protein provided herein.

In yet another aspect, the present invention provides, a method for producing an herbicide-tolerant plant comprising the steps of: a) transforming a plant cell with a recombinant DNA molecule comprising a DNA sequence encoding a transit peptide operably linked to a DNA sequence encoding a heterologous herbicide-tolerance protein, wherein the transit peptide comprises an amino acid sequence comprising at least 97 percent identity to a sequence selected from the group consisting of SEQ ID NOs:4-49 and SEQ ID NOs:236-266; and b) regenerating therefrom an herbicide-tolerant plant that comprises the DNA molecule. In one embodiment, the heterologous herbicide-tolerance protein comprises an amino acid sequence selected from the group consisting of SEQ ID NOs:100-119, SEQ ID NOs:163-182, and SEQ ID NOs:224-228. In another embodiment, the method further comprises the step of crossing the regenerated plant with itself or with a second plant to produce one or more progeny plants. In yet another embodiment, the method may further comprise the step of selecting a progeny plant that is tolerant to at least one PPO herbicide. In certain embodiments, the PPO herbicide is selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, and S-3100.

In a further aspect, the present invention provides a method for producing an herbicide-tolerant transgenic plant or seed comprising crossing a plant comprising a recombinant DNA molecule provided herein with itself or a second plant to produce an herbicide-tolerant transgenic plant or seed. In certain embodiments, the recombinant DNA molecule comprises a DNA sequence encoding a transit peptide operably linked to a DNA sequence encoding a heterologous herbicide-tolerance protein, wherein the transit peptide comprises an amino acid sequence comprising at least 97 percent identity to a sequence selected from the group consisting of SEQ ID NOs:4-49 and SEQ ID NOs:236-266.

In yet a further aspect, the present invention provides a method for expressing a heterologous herbicide-tolerance protein in a plant or cell, the method comprising growing a plant or cell that comprises a recombinant DNA molecule comprising a DNA sequence encoding a transit peptide operably linked to a DNA sequence encoding a heterologous herbicide-tolerance protein, wherein the transit peptide comprises an amino acid sequence comprising at least 97 percent identity to a sequence selected from the group consisting of SEQ ID NOs:4-49 and SEQ ID NOs:236-266, wherein the growing results in expression of the heterologous herbicide-tolerance protein. In one embodiment, the heterologous herbicide-tolerance protein has herbicide-insensitive protoporphyrinogen oxidase activity.

In another aspect, the present invention provides a method for controlling or preventing weed growth in a plant growth area comprising applying an effective amount of at least one PPO herbicide to a plant growth area that comprises a transgenic plant or seed as provided herein, such as a transgenic plant or seed comprising a recombinant DNA molecule comprising a DNA sequence encoding a transit peptide operably linked to a DNA sequence encoding a heterologous herbicide-tolerance protein, wherein the transit peptide comprises an amino acid sequence comprising at least 97 percent identity to a sequence selected from the group consisting of SEQ ID NOs:4-49 and SEQ ID NOs:236-266, wherein the transgenic plant or seed is tolerant to the PPO herbicide. In certain embodiments, the PPO herbicide is selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, and S-3100.

In a further aspect, the present invention provides method for controlling the growth of herbicide tolerant weeds comprising: a) cultivating in a plant growth area a plant or seed provided herein, for instance a plant or seed comprising a recombinant DNA molecule comprising a DNA sequence encoding a transit peptide operably linked to a DNA sequence encoding a heterologous herbicide-tolerance protein, wherein the transit peptide comprises an amino acid sequence comprising at least 97 percent identity to a sequence selected from the group consisting of SEQ ID NOs:4-49 and SEQ ID NOs:236-266; and b) applying a PPO herbicide and at least one other herbicide to the plant growth area, wherein the plant or seed is tolerant to the PPO herbicide and the at least one other herbicide. In certain embodiments, the PPO herbicide is selected from the group consisting of acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, and S-3100. In another embodiment, the other herbicide to which to plant or seed is tolerant is selected from the group consisting of: an ACCase inhibitor, an ALS inhibitor, an EPSPS inhibitor, a synthetic auxin, a photosynthesis inhibitor, a glutamine synthetase inhibitor, a HPPD inhibitor, a PPO inhibitor, and a long-chain fatty acid inhibitor. In further embodiments, the ACCase inhibitor is an aryloxyphenoxy propionate or a cyclohexanedione; the ALS inhibitor is a sulfonylurea, imidazolinone, triazolopyrimidine, or a triazolinone; the EPSPS inhibitor is glyphosate; the synthetic auxin is a phenoxy herbicide, a benzoic acid, a carboxylic acid, or a semicarbazone; the photosynthesis inhibitor is a triazine, a triazinone, a nitrile, a benzothiadiazole, or a urea; the glutamine synthetase inhibitor is glufosinate; the HPPD inhibitor is an isoxazole, a pyrazolone, or a triketone; the PPO inhibitor is a diphenylether, a N-phenylphthalimide, an aryl triazinone, or a pyrimidinedione; or the very long-chain fatty acid inhibitor is a chloroacetamide, an oxyacetamide, or a pyrazole.

In yet a further aspect, the present invention provides a recombinant DNA molecule comprising a DNA sequence encoding a transit peptide operably linked to a DNA sequence encoding a heterologous herbicide-tolerance protein, wherein the transit peptide comprises an amino acid sequence comprising at least 95 percent identity to a sequence selected from the group consisting of SEQ ID NOs:236-266. In one embodiment, the heterologous herbicide-tolerance protein has herbicide-insensitive protoporphyrinogen oxidase activity. In another embodiment, the heterologous herbicide-tolerance protein comprises an amino acid sequence comprising at least 95 percent identity to a sequence selected from the group consisting of SEQ ID NOs:100-119, SEQ ID NOs:163-182, and SEQ ID NOs:224-228. If a further embodiment, the DNA sequence encoding a transit peptide comprises a nucleic acid sequence comprising at least 95 percent identity to a sequence selected from the group consisting of SEQ ID NOs:267-297. In yet another embodiment, the DNA sequence encoding a heterologous herbicide-tolerance protein comprises a nucleic acid sequence comprising at least 95 percent identity to a sequence selected from the group consisting of SEQ ID NOs:121-162 and SEQ ID NOs:183-223, SEQ ID NOs:229-235. In still a further embodiment, the recombinant DNA molecule further comprises a heterologous promoter operably linked to the DNA sequence encoding a transit peptide.

### BRIEF DESCRIPTION OF THE SEQUENCES

SEQ ID NOs:1-2 and SEQ ID NO:236 are amino acid sequences of the Arabidopsis thaliana albino and pale green (APG6) transit peptide.

SEQ ID NO:3 is the amino acid sequence of the cotton 12G088600TP transit peptide.

SEQ ID NOs:4-49 and SEQ ID NOs:237-266 are amino acid sequences of transit peptides.

SEQ ID NOs:50-52 and SEQ ID NO:267 are nucleic acid sequences encoding the APG6 transit peptide.

SEQ ID NO:53 is the nucleic acid sequence encoding the cotton 12G088600TP transit peptide.

SEQ ID NOs:54-99 and SEQ ID NOs:268-297 are exemplary nucleic acid sequences encoding SEQ ID NOs:4-49 and SEQ ID NOs:237-266, respectively.

SEQ ID NOs:100-119 are amino acid sequences of HemG protoporphyrinogen oxidases.

SEQ ID NO:120 is the amino acid sequence of the wild-type protoporphyrinogen oxidase from Amaranthus tuberculatus (waterhemp) (WH).

SEQ ID NOs:121-162 and SEQ ID NO:229 are exemplary nucleic acid sequences encoding SEQ ID NOs:100-119.

SEQ ID NOs:163-182 and SEQ ID NOs:224-228 are amino acid sequences of HemY protoporphyrinogen oxidases.

SEQ ID NOs:183-223 and SEQ ID NOs:230-235 are exemplary nucleic acid sequences encoding SEQ ID NOs:163-182 and SEQ ID NOs:224-228.

### DETAILED DESCRIPTION

The following descriptions and definitions are provided to better define the invention and to guide those of ordinary skill in the art in the practice of the invention. Unless otherwise noted, terms are to be understood according to conventional usage by those of ordinary skill in the relevant art.

Operably linking a transit peptide to a heterologous protein utilizes the transgenic plant cell's protein localization system to achieve sub-cellular localization of the heterologous protein. The transit peptide is removed from the heterologous protein in a processing step during translocation of the heterologous protein into an organelle. The properties of the combination of a specific transit peptide with a specific heterologous protein when expressed in a plant can be unpredictable and surprising. For example, the efficiency of sub-cellular localization and the efficiency of processing (removal of the transit peptide from the heterologous protein) varies and may be affected by the amino acid sequence of the transit peptide, the heterologous protein, or both. These variables affect the function and levels of a heterologous protein and thus affect the phenotype of a transgenic cell, plant, or seed comprising the heterologous protein. Various transit peptides are known in the art for use in transgenic plants, but in view of the variability in the efficiencies of sub-cellular localization and processing and the continuing development of new transgenic traits, novel transit peptides are needed.

The invention provides novel, recombinant DNA molecules for effectively targeting heterologous proteins within plant cells. Effective targeting of a heterologous protein involves efficient sub-cellular localization of the transit peptide and heterologous protein combination and processing of the transit peptide from the heterologous protein. Although transit peptides for localizing heterologous proteins within cells are known, the degree of localization and processing for any transit peptide and heterologous protein combination varies. Localization and processing affect the expression level and function of a heterologous protein and thus affect the phenotype of the cell, plant, or seed comprising the heterologous protein. For example, inefficient localization and processing of a transit peptide and herbicide-tolerance protein combination can result in poor herbicide-tolerance for a transgenic plant.

The invention overcomes these obstacles by providing novel recombinant DNA molecules capable of providing efficient targeting of a protein through improved localization and processing. Recombinant DNA molecules of the invention comprise a DNA sequence encoding a transit peptide operably linked to a DNA sequence encoding a heterologous protein. In one example, recombinant DNA molecules of the invention include, but are not limited to, a recombinant DNA molecule comprising a DNA sequence encoding a transit peptide operably linked to a DNA sequence encoding an herbicide-tolerant protoporphyrinogen oxidase. Compositions and methods for using these recombinant DNA molecules are also provided.

### Recombinant Molecules

As used herein, the term “recombinant” refers to a non-natural DNA, protein, cell, seed, or organism that is the result of genetic engineering and was created by human intervention. A “recombinant DNA molecule” is a DNA molecule that does not naturally occur and as such is the result of human intervention, such as a DNA molecule comprised of a combination of at least two DNA sequences heterologous to each other. An example of a recombinant DNA molecule is a DNA molecule provided herein encoding a transit peptide of the present invention, such as a transit peptide comprising a sequence selected from the group consisting of SEQ ID NOs:4-49 and SEQ ID NOs:236-266, operably linked to a DNA molecule encoding an herbicide-tolerance protein of the present invention, such as a protoporphyrinogen oxidase comprising a sequence selected from the group consisting of SEQ ID NOs:100-119, 163-182, and 224-228. A “recombinant protein” is a protein produced as a result of human intervention that does not naturally occur. An example of a recombinant protein is a protein provided herein comprising a transit peptide of the present invention, such as a transit peptide comprising a sequence selected from the group consisting of SEQ ID NOs:4-49 and SEQ ID NOs:236-266, operably linked to a heterologous protein, such as an herbicide-tolerance protein of the present invention, for instance, a protoporphyrinogen oxidase comprising a sequence selected from the group consisting of SEQ ID NOs:100-119, 163-182, and 224-228. A recombinant cell, seed, or organism is a cell, seed, or organism comprising transgenic or heterologous DNA or protein, for example a transgenic plant cell, seed, plant, or plant part comprising a heterologous DNA molecule or heterologous protein of the invention.

As used herein, the term “isolated DNA molecule” means that the DNA molecule is present alone or in combination with other compositions but is not within its natural environment. A DNA molecule of the invention is an isolated DNA molecule so long as the DNA molecule is not within the DNA of the organism at the genomic location in which it naturally occurs. For example, a recombinant DNA molecule comprising a protein-coding DNA sequence and heterologous transit peptide DNA sequence is considered isolated when it is found in a context that is not the genome in which both the protein-coding DNA sequence and the heterologous transit peptide DNA sequence are naturally found (such as the genome of a transgenic plant, seed, plant part, or cell).

As used herein, the term “genetic engineering” refers to the creation, modification, or production of a DNA molecule, protein, cell, or organism using the techniques of biotechnology (such as molecular biology, protein biochemistry, bacterial transformation, and plant transformation). Genetic engineering is thus a result of human intervention. For example, genetic engineering may be used to create a recombinant DNA molecule encoding a transit peptide comprising a sequence selected from the group consisting of SEQ ID NOs:4-49 and SEQ ID NOs:236-266 operably linked to a DNA molecule encoding an herbicide-tolerance protein, such as a protoporphyrinogen oxidase comprising a sequence selected from the group consisting of SEQ ID NOs:100-119, 163-182, and 224-228 using one or more of the techniques of molecular biology, such as gene cloning, DNA ligation, and DNA synthesis. Such a recombinant DNA molecule optionally may further comprise a heterologous promoter functional in a plant cell.

As used herein, “herbicide-tolerance” or “herbicide-tolerant” with respect to a protein means the ability to maintain at least some of its activity or function in the presence of an herbicide. For example, a protoporphyrinogen oxidase (PPO) is herbicide-tolerant if it maintains at least some of its enzymatic activity in the presence of one or more PPO herbicide(s). Herbicide-tolerance can be measured by any means known in the art. For example, enzymatic activity of a protoporphyrinogen oxidase can be measured by an enzymatic assay in which the production of the product of protoporphyrinogen oxidase or the consumption of the substrate of protoporphyrinogen oxidase in the presence of one or more PPO herbicide(s) is measured via fluorescence, high performance liquid chromatography (HPLC), or mass spectrometry (MS). Another example of an assay for measuring enzymatic activity of a protoporphyrinogen oxidase is a bacterial assay, such as the growth assays described herein, whereby a recombinant protoporphyrinogen oxidase is expressed in a bacterial cell otherwise lacking PPO activity and the ability of the recombinant protoporphyrinogen oxidase to complement this knockout phenotype is measured. Herbicide-tolerance may be complete or partial insensitivity to an herbicide, and may be expressed as a percent (%) tolerance or insensitivity to a PPO herbicide. As used herein, an “herbicide-tolerant protoporphyrinogen oxidase” exhibits herbicide-tolerance in the presence of one or more PPO herbicide(s).

As used herein, “herbicide-tolerance” or “herbicide-tolerant” with respect to an organism, plant, seed, tissue, part, or cell means the organism, plant, seed, tissue, part, or cell's ability to resist the effects of an herbicide when applied. For example, an herbicide-tolerant plant can survive or continue to grow in the presence of the herbicide. The herbicide-tolerance of a plant, seed, plant tissue, plant part, or cell may be measured by comparing the plant, seed, plant tissue, plant part, or cell to a suitable control. For example, the herbicide-tolerance may be measured or assessed by applying an herbicide to a plant comprising a recombinant DNA molecule encoding a protein capable of conferring herbicide-tolerance (the test plant) and a plant not comprising the recombinant DNA molecule encoding the protein capable of conferring herbicide-tolerance (the control plant) and then comparing the plant injury of the two plants, where herbicide-tolerance of the test plant is indicated by a decreased injury rate as compared to the injury rate of the control plant. An herbicide-tolerant plant, seed, plant tissue, plant part, or cells exhibits a decreased response to the toxic effects of an herbicide when compared to a control plant, seed, plant tissue, plant part, or cell. As used herein, an “herbicide-tolerance trait” is a transgenic trait imparting improved herbicide-tolerance to a plant as compared to the wild-type plant. Contemplated plants which might be produced with an herbicide-tolerance trait of the present invention could include, for instance, any plant including crop plants such as soybean (Glycine max), maize (Zea mays), cotton (Gossypium sp.), wheat (Triticum spp.), and Brassica plants, among others.

As used herein, a “hemG knockout strain” means an organism or cell of an organism, such as E. coli, that lacks HemG activity to the extent that it is unable to grow on heme-free growth medium, or such that its growth is detectably impaired in the absence of heme relative to an otherwise isogenic strain comprising a functional HemG. A hemG knockout strain of, for instance, E. coli may be prepared in view of knowledge in the art, for instance in view of the E. coli hemG sequence (Ecogene Accession No. EG11485; Sasarman et al., “Nucleotide sequence of the hemG gene involved in the protoporphyrinogen oxidase activity of Escherichia coli K12” Can J Microbiol 39:1155-1161, 1993).

The term “transgene” refers to a DNA molecule artificially incorporated into an organism's genome as a result of human intervention, such as by plant transformation methods. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. As a result of such genomic alteration, the transgenic plant is something distinctly different from the related wild-type plant and the transgenic trait is a trait not naturally found in the wild-type plant. Transgenic plants of the invention comprise the recombinant DNA molecules provided by the invention.

As used herein, the term “heterologous” refers to the relationship between two or more things not normally associated in nature, for instance that are derived from different sources or not normally found in nature together in any other manner. For example, a DNA molecule or protein may be heterologous with respect to another DNA molecule, protein, cell, plant, seed, or organism if not normally found in nature together or in the same context. In certain embodiments, a first DNA molecule is heterologous to a second DNA molecule if the two DNA molecules are not normally found in nature together in the same context, and a protein is heterologous with respect to a second operably linked protein, such as a transit peptide, if such combination is not normally found in nature. In another embodiment, a recombinant DNA molecule encoding a transit peptide operably linked to a protoporphyrinogen oxidase is heterologous with respect to an operably linked promoter that is functional in a plant cell if such combination is not normally found in nature. A recombinant DNA molecule also may be heterologous with respect to a cell, seed, or organism into which it is inserted when it would not naturally occur in that cell, seed, or organism. A “heterologous protein” is a protein present in a plant, seed, cell, tissue, or organism in which it does not naturally occur or operably linked to a protein with which it is not naturally linked. An example of a heterologous protein is a protein comprising a sequence selected from the group consisting of SEQ ID NOs:4-49, 236-266, 100-119, 163-182, and 224-228 that is expressed in a plant, seed, cell, tissue, or organism in which it does not naturally occur, or that is operably linked to a second protein, such as a transit peptide or herbicide-tolerant protein, with which it is not naturally linked. In another example, a heterologous protein, such as a heterologous herbicide-tolerance protein, for instance a protoporphyrinogen oxidase may be introduced into a plant cell in which it does not naturally occur using the techniques of molecular biology and plant transformation.

As used herein, the term “protein-coding DNA molecule” refers to a DNA molecule comprising a DNA sequence that encodes a protein. As used herein, a “protein-coding DNA sequence” means a DNA sequence that encodes a protein. A protein-coding DNA sequence may be any DNA sequence that encodes a protein, for example a protein comprising a sequence selected from the group consisting of SEQ ID NOs:4-49, 236-266, 100-119, 163-182, and 224-228. As used herein, the term “protein” refers to a chain of amino acids linked by peptide (amide) bonds and includes both polypeptide chains that are folded or arranged in a biologically functional way and polypeptide chains that are not. A “sequence” means a sequential arrangement of nucleotides or amino acids. The boundaries of a protein-coding sequence are usually determined by a translation start codon at the 5′-terminus and a translation stop codon at the 3′-terminus.

As used herein, the term “herbicide-tolerance protein” means a protein capable of conferring herbicide-tolerance to a cell, tissue, plant part, seed, or organism. Examples of herbicide-tolerance proteins are well known in the art and include, but are not limited to, glyphosate-tolerant 5-enolypyruvyl shikimate 3-phosphate synthases (e.g., CP4-EPSPS, 2mEPSPS), glyphosate oxidoreductases (GOX), glyphosate N-acetyltransferases (GAT), herbicide-tolerant acetolactate synthases (ALS)/acetohydroxyacid synthases (AHAS), herbicide-tolerant 4-hydroxyphenylpyruvate dioxygenases (HPPD), dicamba monooxygenases (DMO), phosphinothricin acetyl transferases (PAT), herbicide-tolerant glutamine synthetases (GS), 2,4-dichlorophenoxyproprionate dioxygenases (TfdA), R-2,4-dichlorophenoxypropionate dioxygenases (RdpA), S-2,4-dichlorophenoxypropionate dioxygenases (SdpA), herbicide-tolerant protoporphyrinogen oxidases (PPO), and cytochrome P450 monooxygenases. For example, a protoporphyrinogen oxidase comprising an amino acid sequence selected from the group consisting of SEQ ID NOs:100-119, SEQ ID NOs:163-182, and SEQ ID NOs:224-228 is an herbicide-tolerant protein.

As used herein, “transgene expression”, “expressing a transgene”, “protein expression”, and “expressing a protein” mean the production of a protein through the process of transcribing a DNA molecule into messenger RNA (mRNA) and translating the mRNA into polypeptide chains, which may or may not be ultimately folded into proteins. A protein-coding DNA molecule may be operably linked to a heterologous promoter in a DNA construct for use in expressing the protein in a cell transformed with, and thus comprising, the recombinant DNA molecule or a portion thereof. As used herein, “operably linked” means two DNA or protein molecules linked in manner so that one may affect the function of the other. Operably-linked DNA molecules may be part of a single contiguous molecule and may or may not be adjacent. For example, a promoter is operably linked with a protein-coding DNA molecule in a DNA construct where the two DNA molecules are so arranged that the promoter may affect the expression of the transgene. In another embodiment, two or more protein molecules may be operably linked. For instance, a transit peptide may be operably linked to a heterologous protein, such as an herbicide-tolerant protein.

In one embodiment, the recombinant DNA molecules of the invention include a DNA sequence encoding a protoporphyrinogen oxidase (PPO) operably linked to a transit peptide sequence. As used herein, “protoporphyrinogen oxidase” or “PPO” means an oxidase capable of converting protoporphyrinogen IX to protoporphyrin IX. Such protoporphyrinogen oxidase are known in the art and include, for instance, the protein sequences provided as SEQ ID NOs:100-119, SEQ ID NOs:163-182, and SEQ ID NOs:224-228.

In another embodiment, the recombinant DNA molecules of the invention include a DNA sequence encoding a transit peptide sequence operably linked to a heterologous nucleic acid sequence encoding a protein that has herbicide-tolerant protoporphyrinogen oxidase activity, whereby the transit peptide sequence facilitates localizing the protein molecule within the cell. Transit peptides are also known in the art as signal sequences, targeting sequences, targeting peptides, and localization sequences. An example of a transit peptide is a chloroplast transit peptide (CTP), a mitochondrial targeting sequence (MTS), or a dual chloroplast and mitochondrial targeting peptide. By facilitating protein localization within the cell, such as to the mitochondria or chloroplast, the transit peptide ensures localization of a protein to an organelle for optimal enzyme activity and may increase the accumulation of the protein and protect the protein from proteolytic degradation, and/or enhance the level of herbicide-tolerance, and thereby reduce levels of injury in the transgenic cell, seed, or organism after herbicide application. Upon translocation into the organelle, the transit peptide is typically cleaved from the protein, also referred to as processing. Transit peptide processing may be complete (meaning that the complete transit peptide is cleaved from the amino-terminal end of the protein), incomplete (meaning that one or more amino acids of the transit peptide remain on amino-terminal end of the protein), or result in removal one or more amino acids from the amino-terminal end of the protein. Complete processing of the transit peptide from a protoporphyrinogen oxidase increases the level of protein accumulation, thereby increasing PPO herbicide-tolerance and reducing levels of injury in the transgenic cell, seed, or organism after herbicide application. For example, transit peptides may comprise an amino acid sequence of the present invention, such as those provided by SEQ ID NOs:1-49 and SEQ ID NOs:236-266. Such a transit peptide may be encoded by a nucleic acid sequence of the invention, for instance as provided by SEQ ID NOs:50-99 and SEQ ID NOs:267-297.

Recombinant DNA molecules of the present invention may be synthesized and modified by methods known in the art, either completely or in part, especially where it is desirable to provide sequences useful for DNA manipulation (such as restriction enzyme recognition sites or recombination-based cloning sites), plant-preferred sequences (such as plant-codon usage or Kozak consensus sequences), or sequences useful for DNA construct design (such as spacer or linker sequences). The present invention includes DNA molecules and proteins having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, and at least 99% sequence identity to any of the DNA molecule or protein sequences provided herein as SEQ ID NOs:1-297. As used herein, the term “percent sequence identity” or “% sequence identity” refers to the percentage of identical nucleotides or amino acids in a linear polynucleotide or protein sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, CA), MEGAlign (DNAStar Inc., 1228 S. Park St., Madison, WI 53715), and MUSCLE (version 3.6) (Edgar, Nucleic Acids Research 32(5):1792-7, 2004) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence, or a portion thereof, or to a longer sequence.

As used herein, a “DNA construct” is a recombinant DNA molecule comprising two or more heterologous DNA sequences. DNA constructs are useful for transgene expression and may be comprised in vectors and plasmids. DNA constructs may be used in vectors for transformation, that is the introduction of heterologous DNA into a host cell, to produce transgenic plants and cells, and as such may also be contained in the plastid DNA or genomic DNA of a transgenic plant, seed, cell, or plant part. As used herein, a “vector” means any recombinant DNA molecule that may be used for plant transformation. DNA molecules as set forth in the sequence listing, can, for example, be inserted into a vector as part of a construct having the DNA molecule operably linked to a gene expression element that functions in a plant to affect expression of the protein encoded by the DNA molecule. Methods for constructing DNA constructs and vectors are well known in the art. The components for a DNA construct, or a vector comprising a DNA construct, generally include one or more gene expression elements operably linked to a transcribable DNA sequence, such as the following: a promoter for the expression of an operably linked DNA, an operably linked protein-coding DNA molecule, and a 3′ untranslated region. Gene expression elements useful in practicing the present invention include, but are not limited to, one or more of the following type of elements: promoter, 5′ untranslated region, enhancer, leader, cis-acting element, intron, 3′ untranslated region, and one or more selectable marker transgenes.

The DNA constructs of the invention may include a promoter operably linked to a protein-coding DNA molecule provided by the invention, whereby the promoter drives expression of the heterologous protein molecule. Promoters useful in practicing the present invention include those that function in a cell for expression of an operably linked polynucleotide, such as a bacterial or plant promoter. Plant promoters are varied and well known in the art and include those that are inducible, viral, synthetic, constitutive, temporally regulated, spatially regulated, and/or spatio-temporally regulated.

In one embodiment of the invention, a DNA construct provided herein includes a DNA sequence encoding a transit peptide that is operably linked to a heterologous DNA sequence encoding a protein that has herbicide-tolerant protoporphyrinogen oxidase activity, whereby the transit peptide sequence facilitates localizing the protein within the cell.

As used herein, “control” means an experimental control designed for comparison purposes. For example, a control plant in a transgenic plant analysis is a plant of the same type as the experimental plant (that is, the plant to be tested) but does not contain the transgenic insert, recombinant DNA molecule, or DNA construct of the experimental plant. Examples of control plants useful for comparison with transgenic plants include: for maize plants, non-transgenic LH244 maize (ATCC deposit number PTA-1173); for comparison with transgenic soybean plants: non-transgenic A3555 soybean (ATCC deposit number PTA-10207); for comparison with transgenic cotton plants: non-transgenic Coker 130 (Plant Variety Protection (PVP) Number 8900252); for comparison with transgenic canola or Brassica napus plants: non-transgenic Brassica napus variety 65037 Restorer line (Canada Plant Breeders' Rights Application 06-5517); for comparison with transgenic wheat plants: non-transgenic wheat variety Samson germplasm (PVP 1994).

As used herein, “wild-type” means a naturally occurring similar, but not identical, version. A “wild-type DNA molecule” or “wild-type protein” is a naturally occurring version of the DNA molecule or protein, that is, a version of the DNA molecule or protein pre-existing in nature. An example of a wild-type protein useful for comparison with the engineered proteins provided by the invention is the protoporphyrinogen oxidase from Arabidopsis thaliana. A “wild-type plant” is a non-transgenic plant of the same type as the transgenic plant, and as such is genetically distinct from the transgenic plant comprising the herbicide-tolerance trait. Examples of wild-type plants useful for comparison include: for transgenic maize plants, non-transgenic LH244 maize (ATCC deposit number PTA-1173); for comparison with transgenic soybean plants, non-transgenic A3555 soybean (ATCC deposit number PTA-10207); for comparison with transgenic cotton plants, non-transgenic Coker 130 (Plant Variety Protection Number 8900252); for comparison with transgenic canola or Brassica napus plants, non-transgenic Brassica napus variety 65037 Restorer line (Canada Plant Breeders' Rights Application 06-5517); for comparison with transgenic wheat plants, non-transgenic wheat variety Samson germplasm (PVP 1994).

### Transgenic Plants & Herbicides

An aspect of the invention includes transgenic plant cells, transgenic plant tissues, transgenic plants, and transgenic seeds that comprise the recombinant DNA molecules provided by the invention. These cells, tissues, plants, and seeds comprising the recombinant DNA molecules exhibit tolerance to one or more PPO herbicide(s), and, optionally, tolerance to one or more additional herbicide(s).

Suitable methods for transformation of host plant cells for use with the current invention include virtually any method by which DNA can be introduced into a cell (for example, where a recombinant DNA construct is stably integrated into a plant chromosome) and are well known in the art. Exemplary methods for introducing a recombinant DNA construct into plants include the Agrobacterium transformation system and DNA particle-bombardment, both of which are well known to those of skill in the art. Another exemplary method for introducing a recombinant DNA construct into plants is insertion of a recombinant DNA construct into a plant genome at a pre-determined site by methods of site-directed integration. Site-directed integration may be accomplished by any method known in the art, for example, by use of zinc-finger nucleases, engineered or native meganucleases, TALE-endonucleases, or an RNA-guided endonuclease (for example a CRISPR/Cas9 system). Transgenic plants can be regenerated from a transformed plant cell by the methods of plant cell culture or by taking a cutting from a transgenic plant and rooting the cutting to establish a vegetative clone of the transgenic plant. A transgenic plant homozygous with respect to a transgene (that is, two allelic copies of the transgene) can be obtained by self-pollinating (selfing) a transgenic plant that contains a single transgene allele with itself, for example an R0 plant, to produce R1 seed. One fourth of the R1 seed produced will be homozygous with respect to the transgene. Plants grown from germinating R1 seed can be tested for zygosity, typically using a SNP assay, DNA sequencing, or a thermal amplification assay that allows for the distinction between heterozygotes and homozygotes, referred to as a zygosity assay.

As used herein, “herbicide” is any molecule that is used to control, prevent, or interfere with the growth of one or more plants. Exemplary herbicides include acetyl-CoA carboxylase (ACCase) inhibitors (for example aryloxyphenoxy propionates and cyclohexanediones); acetolactate synthase (ALS) inhibitors (for example sulfonylureas, imidazolinones, triazolopyrimidines, and triazolinones); 5-enolpyruvylshikimate-3-phosphate synthase (EPSPS) inhibitors (for example glyphosate), synthetic auxins (for example phenoxys, benzoic acids, carboxylic acids, semicarbazones), photosynthesis (photosystem II) inhibitors (for example triazines, triazinones, nitriles, benzothiadiazoles, and ureas), glutamine synthetase (GS) inhibitors (for example glufosinate and bialaphos), 4-hydroxyphenylpyruvate dioxygenase (HPPD) inhibitors (for example isoxazoles, pyrazolones, and triketones), protoporphyrinogen oxidase (PPO) inhibitors (for example diphenylethers, N-phenylphthalimide, aryl triazinones, and pyrimidinediones), very long-chain fatty acid inhibitors (for example chloroacetamides, oxyacetamides, and pyrazoles), cellulose biosynthesis inhibitors (for example indaziflam), photosystem I inhibitors (for example paraquat), microtubule assembly inhibitors (for example pendimethalin), and phytoene desaturase (PDS) inhibitors (for example norflurazone), among others.

As used herein, a “PPO herbicide” is a chemical that targets and inhibits the enzymatic activity of a protoporphyrinogen oxidase (PPO), which catalyzes the dehydrogenation of protoporphyrinogen IX to form protoporphyrin IX, which is the precursor to heme and chlorophyll. Inhibition of protoporphyrinogen oxidase causes formation of reactive oxygen species, resulting in cell membrane disruption and ultimately the death of susceptible cells. PPO herbicides are well-known in the art and commercially available. Examples of PPO herbicides include, but are not limited to, diphenylethers (such as acifluorfen, its salts and esters, aclonifen, bifenox, its salts and esters, ethoxyfen, its salts and esters, fluoronitrofen, furyloxyfen, halosafen, chlomethoxyfen, fluoroglycofen, its salts and esters, lactofen, its salts and esters, oxyfluorfen, and fomesafen, its salts and esters); thiadiazoles (such as fluthiacet-methyl and thidiazimin); pyrimidinediones or phenyluracils (such as benzfendizone, butafenacil, ethyl [3-2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS Registry Number 353292-31-6 and referred to herein as S-3100), flupropacil, saflufenacil, and tiafenacil); phenylpyrazoles (such as fluazolate, pyraflufen and pyraflufen-ethyl); oxadiazoles (such as oxadiargyl and oxadiazon); triazolinones (such as azafenidin, bencarbazone, carfentrazone, its salts and esters, and sulfentrazone); oxazolidinediones (such as pentoxazone); N-phenylphthalimides (such as cinidon-ethyl, flumiclorac, flumiclorac-pentyl, and flumioxazin); benzoxazinone derivatives (such as 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione); flufenpyr and flufenpyr-ethyl; pyraclonil; and profluazol. Protoporphyrinogen oxidases and cells, seeds, plants, and plant parts provided by the invention exhibit herbicide-tolerance to one or more PPO herbicide(s).

Plants, seeds, plant parts, plant tissues, and cells provided by the invention exhibit herbicide-tolerance to one or more PPO herbicide(s). PPO herbicide(s) may be applied to a plant growth area comprising the plants and seeds provided by the invention as a method for controlling weeds. Plants and seeds provided by the invention comprise an herbicide-tolerance trait and as such are tolerant to the application of one or more PPO herbicide(s). The herbicide application may be the recommended commercial rate (1×) or any fraction or multiple thereof, such as twice the recommended commercial rate (2×). Herbicide rates may be expressed as grams per hectare (g/h) or pounds per acre (lbs/acre), acid equivalent per pound per acre (lb ae/acre), acid equivalent per gram per hectare (g ae/ha), pounds active ingredient per acre (lb ai/acre), or grams active ingredient per hectare (g ai/ha) depending on the herbicide and the formulation. The herbicide application comprises at least one PPO herbicide. The plant growth area may or may not comprise weed plants at the time of herbicide application. An herbicidally effective dose of PPO herbicide for use in an area for controlling weeds should consist of a range from about 0.1× to about 30× label rate(s) over a growing season. The 1× label rate for some exemplary PPO herbicides is provided in Table 1. One (1) acre is equivalent to 2.47105 hectares and one (1) pound is equivalent to 453.592 grams. Herbicide rates can be converted between English and metric as: (lb ai/ac) multiplied by 1.12=(kg ai/ha) and (kg ai/ha) multiplied by 0.89=(lb ai/ac).

TABLE 1
Exemplary PPO Herbicides

| PPO Herbicide | Chemical Family | 1X Rate | |
|---|---|---|---|
| acifluorfen | Diphenylethers | 420 | g ai/ha |
| fomesafen | Diphenylethers | 420 | g ai/ha |
| lactofen | Diphenylethers | 70-220 | g ai/ha |
| fluoroglycofen-ethyl | Diphenylethers | 15-40 | g ai/ha |
| oxyfluorfen | Diphenylethers | 0.28-2.24 | kg ai/ha |
| flumioxazin | N-phenylphthalimide | 70-105 | g ai/ha |
| azafenidin | Triazolinone | 240 | g ai/ha |
| carfentrazone-ethyl | Triazolinone | 4-36 | g ai/ha |
| sulfentrazone | Triazolinone | 0.1-0.42 | kg ai/ha |
| fluthiacet-methyl | Thiadiazole | 3-15 | g ai/ha |
| oxadiargyl | Oxadiazole | 50-150 | g ai/ha |
| oxadiazon | Oxadiazole | 2.24-4.48 | kg ai/ha |
| pyraflufen-ethyl | Phenylpyrazole | 6-12 | g ai/ha |
| saflufenacil | Pyrimidine dione | 25-100 | g/ha |
| S-3100 | Pyrimidine dione | 5-80 | g/ha |

Herbicide applications may be sequentially or tank mixed with one, two, or a combination of several herbicides or any other compatible herbicide. Multiple applications of one herbicide or of two or more herbicides, in combination or alone, may be used over a growing season to areas comprising transgenic plants of the invention for the control of a broad spectrum of dicot weeds, monocot weeds, or both, for example, two applications (such as a pre-planting application and a post-emergence application or a pre-emergence application and a post-emergence application) or three applications (such as a pre-planting application, a pre-emergence application, and a post-emergence application or a pre-emergence application and two post-emergence applications).

As used herein, a “weed” is any undesired plant. A plant may be considered generally undesirable for agriculture or horticulture purposes (for example, Amaranthus species) or may be considered undesirable in a particular situation (for example, a crop plant of one species in a field of a different species, also known as a volunteer plant).

The transgenic plants, progeny, seeds, plant cells, and plant parts of the invention may also contain one or more additional traits. Additional traits may be introduced by crossing a plant containing a transgene comprising the recombinant DNA molecules provided by the invention with another plant containing one or more additional trait(s). As used herein, “crossing” means breeding two individual plants to produce a progeny plant. Two plants may thus be crossed to produce progeny that contain the desirable traits from each parent. As used herein “progeny” means the offspring of any generation of a parent plant, and transgenic progeny comprise a DNA construct provided by the invention and inherited from at least one parent plant. Additional trait(s) also may be introduced by co-transforming a DNA construct for that additional transgenic trait(s) with a DNA construct comprising the recombinant DNA molecules provided by the invention (for example, with all the DNA constructs present as part of the same vector used for plant transformation) or by inserting the additional trait(s) into a transgenic plant comprising a DNA construct provided by the invention or vice versa (for example, by using any of the methods of plant transformation or genome editing on a transgenic plant or plant cell). Such additional traits include, but are not limited to, increased insect resistance, increased water use efficiency, increased yield performance, increased drought resistance, increased seed quality, improved nutritional quality, hybrid seed production, and herbicide-tolerance, in which the trait is measured with respect to a wild-type plant. Exemplary additional herbicide-tolerance traits may include transgenic or non-transgenic tolerance to one or more herbicides such as ACCase inhibitors (for example aryloxyphenoxy propionates and cyclohexanediones), ALS inhibitors (for example sulfonylureas, imidazolinones, triazolopyrimidines, and triazolinones) EPSPS inhibitors (for example glyphosate), synthetic auxins (for example phenoxys, benzoic acids, carboxylic acids, semicarbazones), photosynthesis inhibitors (for example triazines, triazinones, nitriles, benzothiadiazoles, and ureas), glutamine synthesis inhibitors (for example glufosinate), HPPD inhibitors (for example isoxazoles, pyrazolones, and triketones), PPO inhibitors (for example diphenylethers, N-phenylphthalimide, aryl triazinones, and pyrimidinediones), and long-chain fatty acid inhibitors (for example chloroacetamindes, oxyacetamides, and pyrazoles), among others. Exemplary insect resistance traits may include resistance to one or more insect members within one or more of the orders of Lepidoptera, Coleoptera, Hemiptera, Thysanoptera, Diptera, Hymenoptera, and Orthoptera, among others. Such additional traits are well known to one of skill in the art; for example, and a list of such transgenic traits is provided by the United States Department of Agriculture's (USDA) Animal and Plant Health Inspection Service (APHIS).

A cell transformed with a polynucleotide of the present invention, such as an expression construct, may be selected for the presence of the polynucleotide or its encoded enzymatic activity before or after regenerating such a cell into a transgenic plant. Transgenic plants comprising such a polynucleotide may thus be selected for instance by identifying a transgenic plant that comprises the polynucleotide or the encoded enzymatic activity, and/or displays an altered trait relative to an otherwise isogenic control plant. Such a trait may be, for example, tolerance to a PPO herbicide.

Transgenic plants and progeny that contain a transgenic trait provided by the invention may be used with any breeding methods that are commonly known in the art. In plant lines comprising two or more transgenic traits, the transgenic traits may be independently segregating, linked, or a combination of both in plant lines comprising three or more transgenic traits. Back-crossing to a parental plant and out-crossing with a non-transgenic plant are also contemplated, as is vegetative propagation. Descriptions of breeding methods that are commonly used for different traits and crops are well known to those of skill in the art. To confirm the presence of the transgene(s) in a plant or seed, a variety of assays may be performed. Such assays include, for example, molecular biology assays, such as Southern and northern blotting, PCR, and DNA sequencing; biochemical assays, such as detecting the presence of a protein product, for example, by immunological means (ELISAs and western blots) or by enzymatic function; plant part assays, such as leaf or root assays; and, by analyzing the phenotype of the whole plant. To analyze transit peptide processing in a transgenic plant or seed, assays such as Edman degradation sequencing or mass spectrometry analysis may be performed on the heterologous protoporphyrinogen oxidase protein obtained from the transgenic cell, plant, or seed and the resulting sequence data compared to that of the protoporphyrinogen oxidase protein.

Introgression of a transgenic trait into a plant genotype is achieved as the result of the process of backcross conversion. A plant genotype into which a transgenic trait has been introgressed may be referred to as a backcross converted genotype, line, inbred, or hybrid. Similarly, a plant genotype lacking the desired transgenic trait may be referred to as an unconverted genotype, line, inbred, or hybrid.

As used herein, the term “comprising” means “including but not limited to”.

Having described the invention in detail, it will be apparent that modifications, variations, and equivalent embodiments are possible without departing the scope of the invention defined in the appended claims. Furthermore, it should be appreciated that the examples in the present disclosure are provided as non-limiting examples.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit, and scope of the invention. More specifically, it will be apparent that certain agents which are both chemically and physiologically related may be substituted for the agents described herein with the same or similar result achieved. All such similar substitutes and modifications apparent to those skilled in the art are deemed to be within the spirit, scope, and concept of the invention as defined by the appended claims.

### Example 1: Transit Peptide Discovery

Novel transit peptides were mined from a collection of plant sequence databases. Bioinformatic methods and tools, such as hidden Markov models (HMM), the Pfam database, and basic local alignment search tool (BLAST), were used to identify thousands of EST and genomic sequences predicted to encode proteins known to be localized to the chloroplast and mitochondria in plant cells, such as protoporphyrinogen oxidase and heat shock proteins. These sequences were then analyzed, and the sequence encoding the transit peptide was identified. Thousands of putative transit peptide sequences were identified and assessed for predicted efficacy and comparative sequence diversity. From these, 60 unique transit peptides were selected for cloning and testing in plant cells, with variants produced for some of these (indicated as “var” herein). Table 2 provides the SEQ ID NO corresponding to the protein and nucleotide sequences of each transit peptide and variants thereof.

Recombinant DNA molecules encoding the transit peptides were synthesized using the sequence for each predicted transit peptide. DNA constructs were produced operably linking each transit peptide to a promoter and protein-coding sequence. These DNA constructs were then used to transform plant protoplasts. A protoplast assay was used with transformed plant protoplasts to test transit peptides for the functional activity of an operably linked herbicide-tolerance protein in the presence of the herbicide. Successful candidates were then advanced for plant transformation to enable transgenic plant testing.

TABLE 2
Transit Peptides

| Transit Peptide | PRT SEQ ID NO | DNA SEQ ID NO |
|---|---|---|
| ADADI_1600 | 8 | 58 |
| ALLCE_3035 | 16, 37, 46, 47, 237 | 66, 87, 96, 97, 268 |
| AMACR_2643 | 33 | 83 |
| AMAGR_5230 | 29 | 79 |
| AMAPA_1826 | 12 | 62 |
| AMAPA_4787 | 18 | 68 |
| AMBTR_1537 | 30 | 80 |
| ANDGE_6461 | 26 | 76 |
| BRANA_6036 | 31 | 81 |
| BRANA_9788 | 7 | 57 |
| CAMSA_6215 | 21, 41 | 71, 91 |
| CANRO_3271 | 24 | 74 |
| CANRO_3976 | 4, 35 | 54, 85 |
| CONCA_4103 | 11 | 61 |
| CUCME_4756 | 22, 39, 48 | 72, 89, 98 |
| DIGSA_5107 | 17 | 67 |
| DIGSA_5109 | 27 | 77 |
| ERATE_2090 | 25 | 75 |
| ERATE_4149 | 23, 36, 45 | 73, 86, 95 |
| ERATE_4824 | 28 | 78 |
| KOCSC_1672 | 14 | 64 |
| NICBE_5162 | 6 | 56 |
| ROSHY_6783 | 32 | 82 |
| ROSHY_8873 | 9 | 59 |
| SEDAL_8241 | 20 | 70 |
| SENOB_8832 | 5, 44 | 55, 94 |
| SETIT_2080 | 15 | 65 |
| SPIOL_0401 | 19 | 69 |
| SPIOL_0410 | 13 | 63 |
| TAROF_2111 | 34, 42, 38, 43 | 84, 92, 88, 93 |
| XANST_27 | 10, 40, 49 | 60, 90, 99 |
| ERATE_3481 | 238 | 269 |
| SETIT_9796 | 239 | 270 |
| ACAOS_3432 | 240 | 271 |
| ADADI_0544 | 241 | 272 |
| TAROF_9570 | 242 | 273 |
| AMACR_2380 | 243 | 274 |
| AMACR_2381 | 244 | 275 |
| AMAHY_5254 | 245 | 276 |
| AMAPA_22810 | 246 | 277 |
| AMAPA_2811 | 247 | 278 |
| AMAPA_6265_1 | 248 | 279 |
| AMAPA_6265_2 | 249 | 280 |
| AMAPA_2906 | 250 | 281 |
| AMARU_1762 | 251 | 282 |
| AMARU_1763 | 252 | 283 |
| AMARU_1764 | 253 | 284 |
| AMAVI_1826 | 254 | 285 |
| AMAVI_1827 | 255 | 286 |
| AMBTR_6334 | 256 | 287 |
| CONCA_3910 | 257 | 288 |
| CUCME_3420 | 258 | 289 |
| KOCSC_5431 | 259 | 290 |
| KOCSC_9516 | 260 | 291 |
| KOCSC_0438 | 261 | 292 |
| ROSHY_3269 | 262 | 293 |
| SEDAL_6599 | 263 | 294 |
| SEDAL_6601 | 264 | 295 |
| SPIOL_1551 | 265 | 296 |
| ALLCE_6618 | 266 | 297 |

### Example 2: PPO Enzyme Discovery

Novel microbial HemG and HemY protoporphyrinogen oxidases that are tolerant to PPO herbicides were identified from microbial sequence databases using bioinformatic methods and a novel herbicide bacterial screening system. This screening system used a growth assay of the hemG knockout E. coli strain in LB liquid medium with a PPO herbicide to confirm protoporphyrinogen oxidase activity for an enzyme and to identify protoporphyrinogen oxidases that were not sensitive to the PPO herbicide. Briefly, a hemG knockout E. coli strain was transformed with a bacterial expression vector containing a putative protoporphyrinogen oxidase and cultured in LB liquid medium. Purified crystalline form of one of five different PPO herbicides (acifluorfen (1 mM), flumioxazin (0.5 mM), lactofen (0.5 mM), fomesafen (1 mM), and S-3100 (100 microM), representing three different PPO chemistry subclasses, was added to the medium. Recombinant proteins were expressed and the E. coli growth rates were measured. Growth curves (OD600) were measured for the different variants in the presence and absence of the PPO herbicides at selected time-points from time zero to twenty-four hours. The growth of a transformed hemG knockout E. coli strain in LB medium in the presence of a PPO herbicide indicated that the gene used to transform the E. coli encoded an herbicide-tolerant protoporphyrinogen oxidase. The hemG knockout E. coli strain expressing the waterhemp (WH) protoporphyrinogen oxidase (SEQ ID NO: 120), which is sensitive to all five PPO herbicides, was used as a control to confirm that the assay could distinguish between sensitive and tolerant protoporphyrinogen oxidases for each of the herbicides.

Protoporphyrinogen oxidases that are herbicide-tolerant proteins are provided as SEQ ID NOs:100-119, SEQ ID NOs:163-182, and SEQ ID NOs:224-228 and shown in Table 3. The DNA sequence encoding a protoporphyrinogen oxidase can include at the 5′ end a codon for a methionine, commonly known as a start codon, or this codon (and optionally a few amino-terminal amino acids, for example 2 to 7), can be eliminated to facilitate operable linkage of a transit peptide sequence to the 5′ end of the coding sequence. DNA sequences encoding a protoporphyrinogen oxidase can optionally be synthesized that are optimized for expression in a monocot or dicot. Table 3 provides for each protoporphyrinogen oxidase DNA sequences that are optimized for expression in monocots and dicots.

TABLE 3
Protoporphyrinogen oxidases

| | | | Dicot | Monocot |
|---|---|---|---|---|
| | | Bacterial | optimized | optimized |
| | Protein | DNA SEQ | DNA SEQ | DNA SEQ |
| Name | SEQ ID NO | ID NO | ID NO | ID NO |
| H_N10 | 103, 112 | 124 | 134, 143 | 156 |
| H_N20 | 101, 111 | 122 | 132, 142, 151 | 154 |
| H_N30 | 104, 113 | 125 | 135, 144 | 157 |
| H_N40 | 105, 114 | 126 | 136, 145 | 158 |
| H_N50 | 106, 115 | 127 | 137, 146 | 159 |
| H_N60 | 102 | 123 | 133 | 155 |
| H_N70 | 107 | 128 | 138 | 160 |
| H_N90 | 100, 110, 117, | 121 | 131, 141, 148, | 153 |
| | 118 | | 149, 150, 229 | |
| H_N100 | 108, 116, 119 | 129 | 139, 147, 152 | 161 |
| H_N110 | 109 | 130 | 140 | 162 |
| WH PPO | 120 | n/a | n/a | n/a |
| R2N30 | 163, 164 | 183 | 189, 190 | 195 |
| R2N40 | 165, 224 | 184 | 191, 230 | 196 |
| R2N40opt | 166, 225 | 185 | 231, 232 | n/a |
| R2N70 | 167, 226 | 186 | 192, 233 | 197 |
| R2N90 | 168, 227 | 187 | 193, 234 | 198 |
| R2N100 | 169, 228 | 188 | 194, 235 | 199 |
| R1N473 | 170, 175, 179 | 200 | 205, 216, 220 | 211 |
| R1N533 | 171, 176, 180 | 201 | 206, 217, 221 | 212 |
| R1N171 | 172, 177, 181 | 202 | 207, 218, 222 | 213 |
| R1N311 | 173 | 203 | 208 | 214 |
| R1N333 | 174, 178, 182 | 204 | 209, 210, 219, | 215 |
| | | | 223 | |

### Example 3: Transit Peptide and Protoporphyrinogen Oxidase Testing in Protoplasts

Transit peptides operably linked to a protoporphyrinogen oxidase were tested in plant protoplasts for PPO herbicide-tolerance. Plant transformation vectors were constructed comprising a recombinant DNA molecule encoding the H_N90 protoporphyrinogen oxidase operably linked to a transit peptide. The vectors were then used to transform plant protoplasts, which were assessed for sensitivity to PPO herbicides.

Plant transformation vectors were produced comprising (i) fixed expression elements (a promoter and 3′UTR) operably linked to a transit peptide operably linked to the H_N90 protoporphyrinogen oxidase. Using this, 68 transit peptides were tested and direct comparisons were made by the use of the same protoporphyrinogen oxidase and other expression elements in each vector. Control vectors with the same fixed expression elements were produced comprising (i) H_N90 protoporphyrinogen oxidase without any transit peptide (H_N90 Control) or (ii) Green Fluorescent Protein (GFP) without a transit peptide (GFP Control).

Soybean protoplasts were transformed using standard methods and grown in the presence of the PPO herbicide S-3100 at 1.0 microM concentration. Protoplasts were then assayed for PPO herbicide tolerance, expressed relative to the GFP control (allowing derivation of a relative tolerance score to enable comparisons between experiments). Assays were done in two batches, indicated as Experiment No. 1 or Experiment No. 2. The assays were done in four replications, relative tolerance scores were averaged for each transit peptide, and standard error was calculated (SE). Any targeting peptide scoring a relative tolerance score of 50 or higher was considered highly efficacious for providing efficient sub-cellular localization and processing when operably linked to an herbicide-tolerance protein and a score of 40-50 indicates very good for providing efficient sub-cellular localization and processing when operably linked to an herbicide-tolerance protein. The GFP Control assays had a tolerance score of 0, confirming that the soybean protoplasts were not tolerant to the PPO herbicide in the absence of an herbicide-tolerance protein. The H_N90 Control assays had a tolerance score of 24 (Experiment 1, SE 4) and 11 (Experiment 2, SE 4), while several of the transit peptides provide higher tolerance scores, indicating that an effective transit peptide can increase the herbicide tolerance of the plant protoplasts. For example, ADADI_0544 and KOCSC_9516 scored as highly efficacious targeting peptides and AMAPA_62652 scored as a very good targeting peptide. Data are provided in Table 4.

TABLE 4
Protoplast Assay Results

| Transit Peptide | Tolerance score | SE | Experiment |
|---|---|---|---|
| ADADI_0544 | 62 | 2 | 1 |
| KOCSC_9516 | 60 | 1 | 1 |
| ALLCE_3035_var | 56 | 4 | 1 |
| CAMSA_6215 | 56 | 3 | 1 |
| AMAPA_2810 | 56 | 3 | 1 |
| ALLCE_6618 | 56 | 2 | 1 |
| AMARU_1764 | 56 | 3 | 1 |
| AMBTR_6334 | 56 | 1 | 1 |
| SETIT_9796 | 55 | 5 | 1 |
| AMACR_2381 | 55 | 2 | 1 |
| AMAVI_1827 | 54 | 4 | 1 |
| CONCA_3910 | 54 | 1 | 1 |
| ERATE_3481 | 53 | 2 | 1 |
| ROSHY_3269 | 53 | 5 | 1 |
| AMAPA_6265_1 | 53 | 2 | 1 |
| AMAHY_5254 | 52 | 4 | 1 |
| SEDAL_6599 | 52 | 2 | 1 |
| AMACR_2380 | 51 | 3 | 1 |
| CUCME_3420 | 51 | 3 | 1 |
| AMARU_1762 | 51 | 5 | 1 |
| SEDAL_6601 | 50 | 5 | 1 |
| KOCSC_5431 | 48 | 4 | 1 |
| AMAPA_6265_2 | 47 | 2 | 1 |
| KOCSC_0438 | 47 | 3 | 1 |
| AMAPA_2811 | 46 | 3 | 1 |
| AMAVI_1826 | 45 | 4 | 1 |
| ACAOS_3432 | 44 | 2 | 1 |
| SPIOL_1551 | 43 | 4 | 1 |
| AMAPA_2906 | 43 | 2 | 1 |
| TAROF_9570 | 41 | 3 | 1 |
| AMARU_1763 | 40 | 8 | 1 |
| None - H_N90 Control | 24 | 4 | 1 |
| None - GFP | 0 | 4 | 1 |
| ADADI_0544 | 60 | 1 | 2 |
| SPIOL_1551 | 53 | 3 | 2 |
| KOCSC_9516 | 51 | 4 | 2 |
| ROSHY_3269 | 49 | 4 | 2 |
| AMACR_2381 | 48 | 3 | 2 |
| CAMSA_6215 | 46 | 2 | 2 |
| CUCME_4756_var | 46 | 1 | 2 |
| CUCME_3420 | 46 | 3 | 2 |
| CONCA_3910 | 45 | 4 | 2 |
| AMAGR_5230 | 43 | 2 | 2 |
| SENOB_8832 | 43 | 1 | 2 |
| KOCSC_1672 | 42 | 3 | 2 |
| CONCA_4103 | 36 | 5 | 2 |
| ADADI_1600 | 36 | 4 | 2 |
| BRANA_9788 | 33 | 1 | 2 |
| CUCME_4756 | 33 | 4 | 2 |
| ANDGE_6461 | 33 | 2 | 2 |
| ALLCE_3035 | 33 | 3 | 2 |
| AMAPA_4787 | 30 | 2 | 2 |
| TAROF_2111 | 28 | 3 | 2 |
| ROSHY_6783 | 26 | 4 | 2 |
| CANRO_3976 | 25 | 4 | 2 |
| TAROF_2111_var | 25 | 5 | 2 |
| XANST_27_var | 24 | 2 | 2 |
| NICBE_5162 | 24 | 3 | 2 |
| XANST_27 | 22 | 3 | 2 |
| SPIOL_0401 | 22 | 2 | 2 |
| ERATE_2090 | 22 | 1 | 2 |
| SPIOL_0410 | 21 | 2 | 2 |
| CANRO_3271 | 20 | 2 | 2 |
| AMAPA_1826 | 20 | 2 | 2 |
| DIGSA_5109 | 20 | 2 | 2 |
| DIGSA_5107 | 17 | 2 | 2 |
| ERATE_4149 | 15 | 4 | 2 |
| SETIT_2080 | 14 | 2 | 2 |
| ROSHY_8873 | 12 | 4 | 2 |
| AMBTR_1537 | 12 | 6 | 2 |
| SEDAL_8241 | 11 | 6 | 2 |
| None - H_N90 Control | 11 | 4 | 2 |
| ERATE_4824 | 9 | 5 | 2 |
| ALLCE_3035_var | 8 | 1 | 2 |
| None - GFP | 0 | 4 | 2 |
| AMACR_2643 | 0 | 4 | 2 |

### Example 4: Transit Peptide and Protoporphyrinogen Oxidase Testing in Soybean

Transit peptides operably linked to protoporphyrinogen oxidases were tested in transgenic soybean plants for PPO herbicide-tolerance. Plant transformation vectors were constructed comprising a DNA construct comprising a recombinant DNA molecule optimized for dicot expression and encoding a protoporphyrinogen oxidase operably linked to a transit peptide. The plant transformation vectors were then used to transform soybean, and the plants were regenerated and assessed for their sensitivity to a PPO herbicide.

The genes encoding the seven HemG protoporphyrinogen oxidases H_N10, H_N20, H_N30, H_N40, H_N50, H_N90, and H_N100 were operably linked to thirty-seven different transit peptides and cloned into a base plant transformation vector as described in Example 3. This permitted the side-by-side comparison of seven different HemG protoporphyrinogen oxidases with thirty-seven different transit peptides using the same promoter and 3′UTR elements in every DNA construct. These plant transformation vectors were used to transform soybean excised embryos (germplasm A3555) using A. tumefaciens and standard methods known in the art. Four hundred explants were inoculated for each construct. A sterile PPO herbicide solution was used for herbicide-tolerance testing. The herbicide solution consisted of 0.3 g of S-3100 in crop oil concentrate (5.0 mL) and 495 mL of deionized water.

At five weeks post-transformation, plants were sprayed with two passes of the sterile PPO herbicide solution at a 20 g/ha rate. For each DNA construct tested, four containers each with 30-40 individually transformed plants were tested. The treated plantlets then received at least 15 hours of light exposure post spray each day for four days. At the end of day four post application of S-3100, the treated plantlets were photographed and scored on a visual scale of green coloration (green coloration was representative of healthy photosynthetic plant tissue as compared to photo-bleached tissue) versus damage. The scoring values were 0 for poor tolerance, high damage, low green coloration; 1 for some tolerance, average damage, moderate green coloration; and 2 for good tolerance, low damage, high green coloration. The scoring for each construct is presented in Table 5, where n.d. indicates the analysis was not conducted. The results indicate that several constructs provided tolerance to the PPO herbicide.

TABLE 5
Tolerance score at 5 weeks in soybean

| Transit Peptide | H_N10 | H_N20 | H_N30 | H_N40 | H_N50 | H_N90 | H_N100 |
|---|---|---|---|---|---|---|---|
| APG6 | n.d. | 0 | 2 | 2 | 1 | 2 | 2 |
| 12G088600TP | n.d. | 0 | 0 | 1 | 1 | 2 | 1 |
| CANRO_3976 | 1 | 1 | n.d. | 1 | 1 | 2 | 1 |
| SENOB_8832 | n.d. | 1 | n.d. | 2 | 1 | 1 | n.d. |
| NICBE_5162 | n.d. | n.d. | n.d. | n.d. | 0 | 1 | n.d. |
| BRANA_9788 | 0 | 1 | 0 | n.d. | 2 | 2 | 2 |
| ADADI_1600 | n.d. | 2 | 1 | 2 | 1 | 2 | 2 |
| ROSHY_8873 | 0 | 1 | 1 | 2 | 1 | 0 | 0 |
| XANST_27 | 1 | 1 | 1 | 0 | 1 | 1 | 0 |
| CONCA_4103 | n.d. | n.d. | 0 | n.d. | 1 | 2 | 1 |
| AMAPA_1826 | n.d. | 1 | 1 | 1 | 0 | 2 | 0 |
| SPIOL_0410 | 1 | 2 | 1 | 1 | 1 | 2 | 2 |
| KOCSC_1672 | 1 | 2 | 1 | 1 | 1 | 2 | 0 |
| SETIT_2080 | 0 | 0 | n.d. | 2 | 1 | 2 | 1 |
| ALLCE_3035 | n.d. | 1 | 1 | 2 | 2 | 2 | 2 |
| DIGSA_5107 | 1 | 1 | n.d. | n.d. | 0 | 1 | 1 |
| AMAPA_4787 | n.d. | 2 | 1 | 1 | 1 | 2 | 1 |
| SPIOL_0401 | 1 | 1 | 0 | 1 | 1 | 2 | 1 |
| SEDAL_8241 | 0 | 1 | 0 | 1 | 0 | 1 | 1 |
| CAMSA_6215 | 0 | 2 | n.d. | n.d. | n.d. | 2 | 2 |
| CUCME_4756 | 0 | 0 | n.d. | 2 | 1 | 1 | 0 |
| ERATE_4149 | 1 | 1 | n.d. | n.d. | 2 | 2 | 2 |
| CANRO_3271 | 1 | 1 | n.d. | 1 | 1 | 1 | 2 |
| ERATE_2090 | 0 | 1 | 1 | 0 | 1 | 1 | 0 |
| ANDGE_6461 | n.d. | 1 | n.d. | 2 | 2 | 1 | 1 |
| DIGSA_5109 | 0 | 1 | 0 | 1 | 1 | 0 | n.d. |
| ERATE_4824 | 1 | 1 | 0 | 1 | 1 | 1 | 1 |
| AMAGR_5230 | n.d. | 1 | 0 | 1 | 1 | 2 | 1 |
| AMBTR_1537 | n.d. | 1 | 1 | 1 | 1 | 1 | 1 |
| BRANA_6036 | n.d. | 1 | n.d. | 1 | 1 | 1 | 1 |
| ROSHY_6783 | 1 | 1 | n.d. | 1 | 0 | 0 | 1 |
| AMACR_2643 | n.d. | 0 | n.d. | 1 | 1 | 0 | 2 |
| TAROF_2111 | 1 | 1 | 1 | 0 | 1 | 2 | 1 |
| CANRO_3976_var | n.d. | n.d. | n.d. | n.d. | n.d. | n.d. | n.d. |
| ERATE_4149_var | n.d. | n.d. | n.d. | n.d. | 0 | 2 | 0 |
| ALLCE_3035_var | n.d. | n.d. | n.d. | n.d. | n.d. | 1 | 1 |
| TAROF_2111_var | 0 | n.d. | n.d. | n.d. | 0 | 2 | 1 |
| CUCME_4756_var | n.d. | n.d. | n.d. | n.d. | n.d. | n.d. | n.d. |
| XANST_27_var | n.d. | n.d. | n.d. | n.d. | 0 | n.d. | n.d. |

The plantlets in the non-sprayed containers corresponding to constructs having a score of 2 were then transplanted at approximately seven weeks post-transformation and grown as R0 plants using standard methods known in the art. A selection of plantlets corresponding to non-tolerant scores of 0 and 1 were also grown to serve as negative controls. The R0 plants were grown in a greenhouse under long-day nursery conditions (18 hours of light at 80° F. then 6 hours of dark at 74° F.) for approximately four additional weeks. At eleven weeks post-transformation, the R0 plants were sprayed with two passes of the same herbicide solution described above for a final application rate of 20 g/ha. For each DNA construct tested, 15-30 individually transformed plants were tested. Herbicide injury ratings were visually scored based on the amount of above ground tissue injury with 0% being no visible injury and 100% being complete death of the plant. Non-transgenic control plants scored injury ratings of greater than 300. Marginal tolerance was 30% injury or less, good tolerance is 20% injury or less, and excellent tolerance was considered 10% injury or less. Scores were collected seven days after treatment and averaged for all plants for each DNA construct.

The results of the herbicide-tolerance application at eleven weeks to the R0 plants confirmed the low percent injury rating scores observed at five weeks. For the eleven-week evaluation, any injury rating of 30% or above was equivalent to non-transgenic soybean injury ratings. Several of the constructs stood out as providing very good tolerance to the herbicide application. For example, APG6 (SEQ ID NO: 1) with PPO H_N90 (SEQ ID NO:110) had only 30 injury, APG6 (SEQ ID NO:1) with PPO H_N30 (SEQ ID NO:113) or APG6 (SEQ ID NO:1) with PPO H_N40 (SEQ ID NO:114) each had only 5 injury; transit peptide CAMSA_6215 (SEQ ID NO:21) with PPO H_N90 (SEQ ID NO:110) had only 50 injury. In contrast, transit peptide AMACR_2643 (SEQ ID NO:33) with the PPO H_N90 (SEQ ID NO:110) had an injury score of 5000. Data are provided in Table 6, where n.d. indicates the analysis was not conducted.

TABLE 6
Tolerance score at 11 weeks in soybean

| Transit Peptide | H_N20 | H_N30 | H_N40 | H_N50 | H_N90 | H_N100 |
|---|---|---|---|---|---|---|
| APG6 | n.d. | 5 | 5 | n.d. | 3 | 15 |
| 12G088600TP | n.d. | n.d. | n.d. | n.d. | 35 | n.d. |
| CANRO_3976 | n.d. | n.d. | n.d. | n.d. | 30 | n.d. |
| SENOB_8832 | n.d. | n.d. | 15 | n.d. | n.d. | n.d. |
| NICBE_5162 | n.d. | n.d. | n.d. | n.d. | n.d. | n.d. |
| BRANA_9788 | 25 | n.d. | n.d. | 40 | 25 | 30 |
| ADADI_1600 | 20 | n.d. | 40 | n.d. | 15 | 30 |
| ROSHY_8873 | n.d. | n.d. | 30 | n.d. | 40 | n.d. |
| XANST_27 | n.d. | 35 | n.d. | 40 | 30 | n.d. |
| CONCA_4103 | n.d. | n.d. | n.d. | n.d. | 30 | 35 |
| AMAPA_1826 | n.d. | 35 | n.d. | n.d. | 30 | n.d. |
| SPIOL_0410 | 20 | n.d. | n.d. | n.d. | 30 | 50 |
| KOCSC_1672 | 20 | n.d. | 15 | 40 | 15 | n.d. |
| SETIT_2080 | n.d. | n.d. | 35 | 40 | 25 | n.d. |
| ALLCE_3035 | 30 | 35 | 30 | 40 | 35 | 30 |
| DIGSA_5107 | n.d. | n.d. | n.d. | n.d. | 35 | n.d. |
| AMAPA_4787 | 25 | n.d. | n.d. | 40 | 15 | n.d. |
| SPIOL_0401 | n.d. | n.d. | n.d. | n.d. | 30 | n.d. |
| SEDAL_8241 | n.d. | n.d. | n.d. | n.d. | n.d. | n.d. |
| CAMSA_6215 | 20 | n.d. | n.d. | n.d. | 5 | 35 |
| CUCME_4756 | n.d. | n.d. | 35 | n.d. | 25 | n.d. |
| ERATE_4149 | n.d. | n.d. | n.d. | 40 | 30 | 30 |
| CANRO_3271 | n.d. | n.d. | n.d. | n.d. | 30 | 35 |
| ERATE_2090 | n.d. | n.d. | n.d. | n.d. | n.d. | n.d. |
| ANDGE_6461 | n.d. | n.d. | 15 | 35 | n.d. | n.d. |
| DIGSA_5109 | n.d. | 35 | n.d. | n.d. | 40 | n.d. |
| ERATE_4824 | n.d. | n.d. | n.d. | n.d. | 35 | n.d. |
| AMAGR_5230 | n.d. | n.d. | n.d. | n.d. | 30 | 35 |
| AMBTR_1537 | 30 | n.d. | n.d. | n.d. | n.d. | 40 |
| BRANA_6036 | n.d. | n.d. | n.d. | n.d. | n.d. | n.d. |
| ROSHY_6783 | n.d. | n.d. | n.d. | n.d. | n.d. | n.d. |
| AMACR_2643 | n.d. | n.d. | n.d. | n.d. | 50 | 40 |
| TAROF_2111 | n.d. | n.d. | n.d. | n.d. | 25 | n.d. |
| CANRO_3976_var | n.d. | n.d. | n.d. | n.d. | n.d. | n.d. |
| ERATE_4149_var | n.d. | n.d. | n.d. | n.d. | 35 | n.d. |
| ALLCE_3035_var | n.d. | n.d. | n.d. | n.d. | 15 | 35 |
| TAROF_2111_var | n.d. | n.d. | n.d. | n.d. | 15 | n.d. |
| CUCME_4756_var | n.d. | n.d. | n.d. | n.d. | n.d. | n.d. |
| XANST_27_var | n.d. | n.d. | n.d. | n.d. | n.d. | n.d. |

The genes encoding ten HemY protoporphyrinogen oxidases R2N30, R2N40, R2N40opt, R2N70, R2N90, R2N100, R1N473, R1N533, R1N171, R1N311, and R1N33 were operably linked to thirty-nine different transit peptides and cloned into a base plant transformation vector as described in Example 3. This permitted the side-by-side comparison of ten different HemY protoporphyrinogen oxidases with thirty-nine different transit peptides using the same promoter and 3′UTR elements in every DNA construct. These plant transformation vectors were used to transform soybean excised embryos (germplasm A3555) using A. tumefaciens and standard methods known in the art. Four hundred explants were inoculated for each construct. A sterile PPO herbicide solution was used for herbicide-tolerance testing. The herbicide solution consisted of 0.3 g of 5-3100 in crop oil concentrate (5.0 mL) and 495 mL of deionized water.

At five weeks post-transformation, for each DNA construct four containers (each with 30-40 individually transformed plants) were sprayed with two passes of the sterile PPO herbicide solution for a final application rate of 20 g/ha. The treated plantlets then received at least 15 hours of light exposure post spray each day for four days. At the end of day four post application of S-3100, the treated plantlets were photographed and scored on a visual scale of green coloration (green coloration was representative of healthy photosynthetic plant tissue as compared to photo-bleached tissue) versus damage. The scoring values were 0 for poor tolerance, high damage, low green coloration; 1 for some tolerance, average damage, moderate green coloration; and 2 for good tolerance, low damage, high green coloration. The scoring for each construct is presented in Table 7, where n.d. indicates the analysis was not conducted. The results indicate that several constructs provided tolerance to the PPO herbicide.

TABLE 7
Tolerance score at 5 weeks in soybean

| Transit Peptide | R1N171 | R1N473 | R1N533 | R2N30 | R2N40 | R2N40opt | R2N70 | R2N90 | R2N100 | R1N333 |
|---|---|---|---|---|---|---|---|---|---|---|
| APG6 | 0 | 2 | 0 | 2 | n.d. | 1 | n.d. | n.d. | 0 | n.d. |
| 12G088600TP | 0 | 0 | 2 | n.d. | n.d. | n.d. | 2 | 0 | 0 | 0 |
| CANRO_3976 | 0 | 1 | 0 | 1 | n.d. | n.d. | 1 | n.d. | 0 | 0 |
| SENOB_8832 | n.d. | 1 | 0 | 2 | n.d. | 0 | 0 | n.d. | 0 | 0 |
| NICBE_5162 | 1 | n.d. | n.d. | n.d. | 1 | 1 | n.d. | 0 | 0 | n.d. |
| BRANA_9788 | n.d. | 1 | 1 | n.d. | n.d. | 1 | 0 | n.d. | 0 | 0 |
| ADADI_1600 | 0 | 1 | 0 | 1 | n.d. | 2 | n.d. | n.d. | n.d. | 0 |
| ROSHY_8873 | 1 | 1 | n.d. | 2 | 0 | 1 | 0 | 1 | 1 | 0 |
| XANST_27 | 1 | 1 | n.d. | 2 | 0 | 0 | n.d. | 1 | n.d. | 1 |
| CONCA_4103 | 1 | 1 | 1 | 2 | n.d. | n.d. | n.d. | 0 | 1 | n.d. |
| AMAPA_1826 | 0 | 0 | 0 | 2 | n.d. | 1 | n.d. | n.d. | n.d. | 0 |
| SPIOL_0410 | 0 | 1 | 0 | 1 | n.d. | 2 | n.d. | 1 | 0 | 1 |
| KOCSC_1672 | 0 | 0 | 0 | n.d. | n.d. | 0 | n.d. | 0 | n.d. | 0 |
| SETIT_2080 | n.d. | 1 | 1 | 1 | n.d. | n.d. | n.d. | 0 | 1 | 0 |
| ALLCE_3035 | 1 | 1 | 1 | 2 | n.d. | 1 | n.d. | n.d. | 0 | 0 |
| DIGSA_5107 | 1 | 1 | 2 | 2 | n.d. | 1 | 0 | 0 | n.d. | 0 |
| AMAPA_4787 | 0 | 1 | n.d. | 1 | n.d. | 1 | n.d. | n.d. | 0 | 0 |
| SPIOL_0401 | 0 | 0 | 0 | 1 | n.d. | 0 | n.d. | 1 | 1 | 0 |
| SEDAL_8241 | 1 | 0 | 1 | n.d. | 2 | 1 | n.d. | 1 | 1 | 0 |
| CAMSA_6215 | 0 | 1 | 1 | 2 | n.d. | 1 | n.d. | 0 | n.d. | n.d. |
| CUCME_4756 | 0 | 0 | n.d. | 1 | n.d. | n.d. | 0 | 1 | 0 | 0 |
| ERATE_4149 | n.d. | 1 | 2 | 1 | n.d. | n.d. | n.d. | 0 | 0 | 0 |
| CANRO_3271 | 1 | 1 | 1 | 1 | n.d. | n.d. | n.d. | 1 | 0 | 1 |
| ERATE_2090 | n.d. | 0 | 2 | 2 | n.d. | n.d. | n.d. | 0 | 0 | 0 |
| ANDGE_6461 | 0 | 1 | 0 | 2 | n.d. | 1 | n.d. | n.d. | 0 | 0 |
| DIGSA_5109 | 1 | 0 | 1 | 1 | n.d. | 1 | n.d. | n.d. | 1 | 0 |
| ERATE_4824 | 0 | 1 | 0 | n.d. | n.d. | 2 | n.d. | 0 | 0 | 1 |
| AMAGR_5230 | 0 | 2 | 0 | 2 | n.d. | n.d. | n.d. | 0 | 1 | 0 |
| AMBTR_1537 | 0 | 0 | 1 | 1 | n.d. | 0 | n.d. | 0 | 0 | 1 |
| BRANA_6036 | 1 | 1 | n.d. | 1 | n.d. | 0 | n.d. | 0 | 0 | 0 |
| ROSHY_6783 | 1 | 1 | 1 | 1 | 0 | 0 | 1 | 0 | 1 | 1 |
| AMACR_2643 | 0 | 1 | 1 | 1 | n.d. | 0 | n.d. | 0 | 0 | 0 |
| TAROF_2111 | 0 | 2 | 0 | n.d. | 2 | 1 | 0 | 0 | 0 | 0 |
| CANRO_3976_var | n.d. | n.d. | n.d. | 0 | 1 | n.d. | n.d. | n.d. | n.d. | 1 |
| ERATE_4149_var | 0 | 0 | 1 | 1 | 1 | 1 | n.d. | n.d. | n.d. | n.d. |
| ALLCE_3035_var | n.d. | n.d. | 0 | 1 | 1 | n.d. | n.d. | n.d. | 0 | 1 |
| TAROF_2111_var | 0 | 0 | 0 | 1 | 1 | 2 | n.d. | n.d. | 0 | n.d. |
| CUCME_4756_var | n.d. | n.d. | 2 | n.d. | 2 | n.d. | n.d. | n.d. | n.d. | n.d. |
| XANST_27_var | 1 | 1 | 2 | 1 | 2 | 1 | n.d. | n.d. | n.d. | n.d. |

The plantlets in the non-sprayed containers corresponding to constructs having a score of 2 were then transplanted at approximately seven weeks post-transformation and grown as R0 plants using standard methods known in the art. A selection of plantlets corresponding to non-tolerant scores of 0 and 1 were also grown to serve as negative controls. The R0 plants were grown in a greenhouse under long-day nursery conditions (18 hours of light at 80° F. then 6 hours of dark at 74° F.) for approximately four additional weeks. At eleven weeks post-transformation, the R0 plants were sprayed with two passes of the same herbicide solution described above for a final application rate of 20 g/ha. For each DNA construct tested, 15-30 individually transformed plants were tested. Herbicide injury ratings were visually scored based on the amount of above ground tissue injury with 0% being no visible injury and 100% being complete death of the plant. Non-transgenic control plants scored injury ratings of greater than 30%. Marginal tolerance was 30% injury or less, good tolerance is 20% injury or less, and excellent tolerance was considered 10% injury or less. Scores were collected seven days after treatment and averaged for all plants for each DNA construct.

The results of the herbicide-tolerance application at eleven weeks to the R0 plants confirmed the low percent injury rating scores observed at five weeks. For the eleven-week evaluation, any injury rating of 30 or above was equivalent to non-transgenic soybean injury ratings. A few of the constructs stood out as providing very good tolerance to the herbicide application. For example, transit peptide ANDGE_6461 (SEQ ID NO:26) with R2N30 (SEQ ID NO:163) had only 7 injury. Data are provided in Table 8, where n.d. indicates the analysis was not conducted.

TABLE 8
Tolerance score at 11 weeks in soybean

| Transit Peptide | R1N171 | R1N473 | R1N533 | R2N30 | R2N40 | R2N40opt | R2N70 | R1N333 |
|---|---|---|---|---|---|---|---|---|
| APG6 | n.d. | 30 | n.d. | 17 | n.d. | 20 | n.d. | n.d. |
| 12G088600TP | n.d. | n.d. | 40 | n.d. | n.d. | n.d. | 30 | n.d. |
| CANRO_3976 | n.d. | n.d. | n.d. | n.d. | n.d. | n.d. | n.d. | n.d. |
| SENOB_8832 | n.d. | n.d. | n.d. | 25 | n.d. | n.d. | n.d. | n.d. |
| NICBE_5162 | n.d. | n.d. | n.d. | n.d. | n.d. | n.d. | n.d. | n.d. |
| BRANA_9788 | n.d. | 35 | n.d. | n.d. | n.d. | n.d. | n.d. | n.d. |
| ADADI_1600 | n.d. | n.d. | n.d. | 25 | n.d. | 30 | n.d. | n.d. |
| ROSHY_8873 | n.d. | n.d. | n.d. | 35 | n.d. | 30 | n.d. | 35 |
| XANST_27 | n.d. | n.d. | n.d. | 20 | n.d. | 25 | n.d. | 35 |
| CONCA_4103 | n.d. | n.d. | n.d. | 25 | n.d. | n.d. | n.d. | n.d. |
| AMAPA_1826 | n.d. | n.d. | n.d. | 25 | n.d. | n.d. | n.d. | n.d. |
| SPIOL_0410 | n.d. | n.d. | n.d. | n.d. | n.d. | 35 | n.d. | n.d. |
| KOCSC_1672 | n.d. | n.d. | n.d. | n.d. | n.d. | n.d. | n.d. | n.d. |
| SETIT_2080 | n.d. | n.d. | n.d. | 20 | n.d. | n.d. | n.d. | 35 |
| ALLCE_3035 | n.d. | n.d. | n.d. | 25 | n.d. | n.d. | n.d. | n.d. |
| DIGSA_5107 | 30 | 40 | 35 | 35 | n.d. | n.d. | n.d. | n.d. |
| AMAPA_4787 | n.d. | n.d. | n.d. | 25 | n.d. | n.d. | n.d. | n.d. |
| SPIOL_0401 | n.d. | n.d. | n.d. | 15 | n.d. | n.d. | n.d. | n.d. |
| SEDAL_8241 | n.d. | n.d. | n.d. | n.d. | 20 | n.d. | n.d. | n.d. |
| CAMSA_6215 | n.d. | n.d. | n.d. | 15 | n.d. | 20 | n.d. | n.d. |
| CUCME_4756 | n.d. | n.d. | n.d. | n.d. | n.d. | n.d. | n.d. | n.d. |
| ERATE_4149 | n.d. | n.d. | 35 | 25 | n.d. | n.d. | n.d. | n.d. |
| CANRO_3271 | n.d. | n.d. | n.d. | n.d. | n.d. | n.d. | n.d. | n.d. |
| ERATE_2090 | n.d. | n.d. | 35 | 15 | n.d. | n.d. | n.d. | n.d. |
| ANDGE_6461 | n.d. | n.d. | n.d. | 7 | n.d. | n.d. | n.d. | n.d. |
| DIGSA_5109 | n.d. | n.d. | n.d. | n.d. | n.d. | n.d. | n.d. | n.d. |
| ERATE_4824 | n.d. | n.d. | n.d. | n.d. | n.d. | 25 | n.d. | n.d. |
| AMAGR_5230 | n.d. | 35 | n.d. | 35 | n.d. | n.d. | n.d. | n.d. |
| AMBTR_1537 | n.d. | n.d. | n.d. | n.d. | n.d. | n.d. | n.d. | n.d. |
| BRANA_6036 | n.d. | n.d. | n.d. | 25 | n.d. | n.d. | n.d. | n.d. |
| ROSHY_6783 | n.d. | n.d. | n.d. | n.d. | n.d. | n.d. | n.d. | n.d. |
| AMACR_2643 | n.d. | n.d. | n.d. | n.d. | n.d. | n.d. | n.d. | n.d. |
| TAROF_2111 | n.d. | 40 | n.d. | n.d. | 20 | n.d. | n.d. | n.d. |
| CANRO_3976_var | n.d. | n.d. | n.d. | n.d. | n.d. | n.d. | n.d. | n.d. |
| ERATE_4149_var | n.d. | n.d. | n.d. | n.d. | n.d. | n.d. | n.d. | n.d. |
| ALLCE_3035_var | n.d. | n.d. | n.d. | 25 | n.d. | n.d. | n.d. | n.d. |
| TAROF_2111_var | n.d. | n.d. | n.d. | n.d. | n.d. | 25 | n.d. | n.d. |
| CUCME_4756_var | n.d. | n.d. | 35 | n.d. | 25 | n.d. | n.d. | n.d. |
| XANST_27_var | n.d. | 30 | 35 | n.d. | n.d. | n.d. | n.d. | n.d. |

The genes encoding the HemG protoporphyrinogen oxidase H_N90 was operably linked to 44 different transit peptides and cloned into a base plant transformation vector as described in Example 3. This permitted the side-by-side comparison of different transit peptides using the same promoter, herbicide-tolerance protein, and 3′UTR elements in every DNA construct. These plant transformation vectors were used to transform soybean excised embryos (germplasm AG3555) using A. tumefaciens and standard methods known in the art. Four hundred to 4,5000 individual transgenic plants were tested for each construct. A sterile PPO herbicide solution was used for herbicide-tolerance testing. The herbicide solution consisted of 0.3 g of 5-3100 in crop oil concentrate (5.0 mL) and 495 mL of deionized water.

At five weeks post-transformation, plants were sprayed with two passes of the sterile PPO herbicide solution for a final application rate of 20 g/ha. For each DNA construct tested, 400 to 4,5000 replications were done. The treated plantlets then received at least 15 hours of light exposure post spray each day for four days. At the end of day four post-application of S-3100, the treated plantlets were scored for percentage of relative pass frequency (defined as the percentage of all the individual plants for a DNA construct that visually display tolerance to the herbicide application relative to control transgenic plants sprayed with a surfactant only solution.). Plantlets in the non-sprayed containers were transplanted at approximately seven weeks post-transformation and grown as R0 plants. The R0 plants were grown in a greenhouse under long-day nursery conditions (18 hours of light at 80° F. then 6 hours of dark at 74° F.) for approximately four additional weeks. At eleven to twelve weeks post-transformation, the R0 plants were sprayed with two passes of the same herbicide solution described above at a 20 g/ha rate. For each DNA construct tested, 15-45 replications were done. Herbicide injury ratings were collected three to seven days after treatment. For the eleven-week evaluation, the percentage of plants at or below 10% injury and at or below 20% injury was recorded. At the herbicide application rates tested, transgenic plants expressing the protoporphyrinogen oxidase H_N90 without any operably linked transit peptide (PPO Control), produced a zero plants with 20% injury or less. Several of the transit peptides operably linked to the H_N90 herbicide tolerance protein stood out as providing excellent or very good tolerance to the herbicide application. For example, at the eleven-week spray over 50% of plants had an injury score at or below 20% when expressing H-N90 operably linked to ALLCE_3035 (57%), KOCSC_9516 (59%), CAMSA_6215 (69%), ROSHY_3269 (70%), ADADI_0544 (75%), CUCME_3420 (80%), SPIOL_1551 (85%), CUCME_4756 (89%), or CONCA_3910 (900%). Data are provided in Table 9.

TABLE 9
Tolerance score at 5 and 11 weeks in soybean

| | 5 week spray | 11 week spray | 11 week spray |
|---|---|---|---|
| | relative pass | % plants | % plants |
| Transit Peptide | frequency | at ≤10% | at ≤20% |
| CUCME_4756 | 27% | 0% | 0% |
| CANRO_3271 | 23% | 0% | 0% |
| DIGSA_5109 | 24% | 0% | 0% |
| CAMSA_6215 | 68% | 62% | 69% |
| AMACR_2381 | 30% | 0% | 0% |
| ROSHY_3269 | 54% | 25% | 70% |
| CUCME_3420 | 51% | 20% | 80% |
| ADADI_0544 | 30% | 20% | 75% |
| SPIOL_1551 | 40% | 70% | 85% |
| NICBE_5162 | 9% | 0% | 0% |
| CUCME_4756 | 28% | 26% | 89% |
| BRANA_9788 | 11% | 0% | 0% |
| SPIOL_0410 | 18% | 0% | 0% |
| XANST_0027 | 22% | 0% | 0% |
| SETIT_2080 | 3% | 0% | 0% |
| ERATE_4149 | 3% | 0% | 0% |
| TAROF_2111 | 3% | 0% | 0% |
| CONCA_4103 | 26% | 0% | 0% |
| CANRO_3976 | 6% | 0% | 0% |
| AMACR_2643 | 3% | 0% | 0% |
| SPIOL_0401 | 6% | 0% | 0% |
| ADADI_1600 | 30% | 0% | 0% |
| ANDGE_6461 | 47% | 0% | 0% |
| ERATE_2090 | 11% | 0% | 0% |
| 12G088600TP | 13% | 0% | 0% |
| ALLCE_3035 | 5% | 0% | 0% |
| SENOB_8832 | 52% | 0% | 0% |
| TAROF_2111 | 66% | 0% | 0% |
| ROSHY_8873 | 10% | 0% | 0% |
| KOCSC_1672 | 25% | 12% | 24% |
| AMBTR_1537 | 2% | 0% | 0% |
| AMAPA_1826 | 7% | 0% | 0% |
| BRANA_6036 | 5% | 0% | 0% |
| CONCA_3910 | 40% | 60% | 90% |
| AMAPA_4787 | 6% | 0% | 0% |
| ROSHY_6783 | 0% | 0% | 0% |
| ALLCE_3035 | 26% | 35% | 57% |
| ERATE_4824 | 12% | 0% | 0% |
| AMAGR_5230 | 2% | 0% | 0% |
| SEDAL_8241 | 5% | 0% | 0% |
| DIGSA_5107 | 11% | 0% | 0% |
| KOCSC_9516 | 27% | 16% | 59% |
| XANST_0027_var | 3% | 0% | 0% |
| APG6 | 60% | 30% | 63% |
| None - PPO Control | 1% | 0% | 0% |

### Example 5: Transit Peptide and Protoporphyrinogen Oxidase Testing in Corn

Transit peptides operably linked to protoporphyrinogen oxidases were tested in transgenic corn plants for PPO herbicide-tolerance. Plant transformation vectors were constructed comprising a DNA construct comprising a recombinant DNA molecule optimized for monocot expression and encoding a protoporphyrinogen oxidase operably linked to a transit peptide. The plant transformation vectors were then used to transform corn, and the regenerated plants were assessed for their sensitivity to a PPO herbicide.

The genes encoding the protoporphyrinogen oxidase H_N90 was operably linked to fourteen different transit peptides and cloned into base plant transformation vectors with a variety of promoters and 3′ UTR elements. The use of the same protoporphyrinogen oxidase in each DNA construct permitted the side-by-side comparison of different transit peptides. A plant transformation vector was also produced with the protoporphyrinogen oxidase H_N90 without any operably linked transit peptide (PPO Control). These plant transformation vectors were used to transform corn using A. tumefaciens and standard methods known in the art. Regenerated R0 plants were grown and then screened to access the degree of tolerance exhibited to applications of S-3100 (40 to 80 g/ha rate) at approximately 10-14 weeks post-transformation. Tolerance was visually accessed 3 to 10 days following application of the herbicide. Sprayed plants are scored on the percent of injury to the entire above-ground part of the plant following herbicide treatment, relative to controls. For each DNA construct tested, 10 to 120 plants were tested and the injury rate was averaged. The percentage of R0 plants passing at a 20% injury or less score was recorded. Any DNA construct producing transgenic plants with 50% or more having 20% or less injury was considered a highly tolerant DNA construct. Any DNA construct producing transgenic plants with 20% or more having 20% or less injury was considered a tolerant DNA construct. At the herbicide application rates tested (S-3100 at 40 to 80 g/ha), transgenic plants expressing the protoporphyrinogen oxidase H_N90 without any operably linked transit peptide (PPO Control), with XANST_27 or with ALLCE_3035 produced zero plants with 20% injury or less. However, several of the transit peptides produced transgenic plants expressing the protoporphyrinogen oxidase H_N90 that were highly tolerant or tolerant: ADADI_0544 (41%), ANDGE_6461 (60%), CAMSA_6215 (60% and 41% pass), CONCA_3910 (36% and 45%), ROSHY_3269 (64% and 74%), SPIOL 1551 (50% and 55%), SETIT_9796 (55%). Data are provided in Table 10.

TABLE 10
Tolerance score in corn

| | | | Percent with 20% |
|---|---|---|---|
| Promoter | Transit Peptide | 3′UTR | or less injury |
| A | SETIT_9796 | E | 55% |
| A | ACAOS_3432 | E | 37% |
| A | ADADI_0544 | E | 41% |
| A | TAROF_9570 | E | 29% |
| A | ALLCE_6618 | E | 31% |
| D | ROSHY_3269 | H | 74% |
| B | ROSHY_3269 | F | 64% |
| D | CONCA_3910 | H | 36% |
| B | CONCA_3910 | F | 45% |
| D | SPIOL_1551 | H | 55% |
| B | SPIOL_1551 | F | 50% |
| D | CAMSA_6215 | H | 41% |
| B | CAMSA_6215 | F | 60% |
| B | ANDGE_6461 | F | 60% |
| B | ADADI_1600 | F | 11% |
| D | XANST_27_var | H | 0% |
| C | XANST_27_var | G | 0% |
| A | ALLCE_3035 | E | 0% |
| B | ALLCE_3035 | F | 0% |
| C | None - PPO Control | G | 0% |

### Example 6: Transit Peptide and Protoporphyrinogen Oxidase Testing in Cotton

Transit peptides operably linked to protoporphyrinogen oxidases were tested in transgenic cotton plants for PPO herbicide-tolerance. Plant transformation vectors were constructed comprising a DNA construct comprising a recombinant DNA molecule optimized for dicot expression and encoding a protoporphyrinogen oxidase operably linked to a transit peptide. The plant transformation vectors were then used to transform cotton, and the regenerated plants were assessed for their sensitivity to a PPO herbicide.

The genes encoding the protoporphyrinogen oxidases H_N20 and H_N90 were operably linked to four different transit peptides and cloned into a base plant transformation vector as described in Example 3. This permitted the side-by-side comparison of different transit peptides using the same promoter and 3′UTR elements in every DNA construct. These plant transformation vectors were used to transform cotton using A. tumefaciens and standard methods known in the art. Regenerated plants were grown and then screened to access the degree of tolerance exhibited to applications of S-3100 (20 g/ha rate) at approximately 11 to 12 weeks post-transformation. Tolerance was visually accessed 3 to 10 days following application of the herbicide. Sprayed plants are scored on the percent of injury to the entire above-ground part of the plant following herbicide treatment, relative to controls. For each DNA construct tested, 10-15 replications were tested and the average injury rate was averaged. An average injury score of 50% or less was considered a highly herbicide-tolerant DNA construct, and an average injury score greater than 50% but less than 80% was considered a marginally herbicide-tolerant DNA construct. An average injury score at or above 80% was considered indistinguishable from control plants. Transgenic cotton plants expressing the protoporphyrinogen oxidase H_N90 operably linked to CAMSA_6215 produced plants that were highly herbicide-tolerant with an average injury score of 38%. Transgenic cotton plants expressing the protoporphyrinogen oxidase H_N90 operably linked to AMAPA_4787 produced plants that were marginally herbicide-tolerant with an average injury score of 63%.

1.-14. (canceled)

15. A recombinant protein comprising in operable linkage:
a) a transit peptide comprising an amino acid sequence comprising at least 95 percent identity to a sequence selected from the group consisting of SEQ ID NOs:4-49 and SEQ ID NOs:236-266; and
b) a heterologous herbicide-tolerance protein.

16. The recombinant protein of claim 15, wherein the heterologous herbicide-tolerance protein has herbicide-insensitive protoporphyrinogen oxidase activity.

17. A transgenic plant, seed, or cell comprising the recombinant protein of claim 15.

18. (canceled)

19. (canceled)

20. The recombinant protein of claim 15, wherein the heterologous herbicide-tolerance protein comprises an amino acid sequence having at least 95 percent identity to a sequence selected from the group consisting of SEQ ID NOs:100-119, SEQ ID NOs:163-182, and SEQ ID NOs:224-228.

21.-37. (canceled)
